(19) United States
(12) Patent Application Publication
(10) Pub. No.: US 2018/0244784 A1
(43) Pub. Date: Aug. 30, 2018

(54) VEGFR2 ANTIBODIES

(71) Applicants: Heman Lap Man Chao, Aurora (CA); Wah Yau Wong, Edmonton (CA); Baomin Tian, Edmonton (CA); Marni Diane Uger, Richmond Hill (CA)

(72) Inventors: Heman Lap Man Chao, Aurora (CA); Wah Yau Wong, Edmonton (CA); Baomin Tian, Edmonton (CA); Marni Diane Uger, Richmond Hill (CA)

(21) Appl. No.: 15/862,006

(22) Filed: Jan. 4, 2018

Related U.S. Application Data
(60) Provisional application No. 62/442,637, filed on Jan. 5, 2017.
(60) Provisional application No. 62/480,705, filed on Apr. 3, 2017.
(60) Provisional application No. 62/491,657, filed on Apr. 28, 2017.
(60) Provisional application No. 62/535,325, filed on Jul. 21, 2017.

(51) Int. Cl. C07K 16/28; A61K 47/68; A61K 38/50

(52) U.S. Cl. CPC C07K 16/2863; A61K 47/6849; A61K 47/6803; A61K 38/50; C07K 2317/569; C07K 2317/52; C07K 2317/92; C07K 2317/24; C07K 2317/22; C07K 2317/21; C07K 2319/00

(57) ABSTRACT

Single domain anti-VEGFR-2 antibodies and fragments thereof and variants thereof and uses thereof, for example, for use to inhibit/decrease angiogenesis and induce tumor regression.

### CROSS REFERENCE TO RELATED APPLICATIONS

This application is entitled to priority under 35 U.S.C. § 119(e) to U.S. Provisional Patent Application Ser. No. 62/442,637 filed on Jan. 5, 2017, U.S. Provisional Patent Application Ser. No. 62/480,705 filed on Apr. 3, 2017, U.S. Provisional Patent Application Ser. No. 62/491,657 filed on Apr. 28, 2017 and U.S. Provisional Patent Application Ser. No. 62/535,325 filed on Jul. 21, 2017, each of which application is hereby incorporated herein by reference in its entirety.

### FIELD OF THE INVENTION

The present invention relates generally to the fields of antibodies, angiogenesis and tumor treatment. More particularly, it relates to anti-VEGFR-2 antibodies and fragments thereof and uses thereof, for example, for use to inhibit/decrease angiogenesis and induce tumor regression.

### BACKGROUND OF THE INVENTION

Angiogenesis is required for invasive tumor growth and metastasis and constitutes an important point in the control of cancer progression. Tumor angiogenesis is mediated by tumor-secreted angiogenic growth factors that interact with their surface receptors expressed on endothelial cells. Avascular tumors are severely restricted in their growth potential because of the lack of a blood supply. An “angiogenic switch” allows tumors to vascularize and develop in size and metastatic potential through perturbing the local balance of proangiogenic and antiangiogenic factors. Frequently, tumors overexpress proangiogenic factors, such as vascular endothelial growth factor, allowing them to make this angiogenic switch.

Vascular endothelial growth factor. VEGF, is an endothelial cell-specific mitogen. It is distinct among growth factors in that it acts as an angiogenesis inducer by specifically promoting the proliferation of endothelial cells. The biological response of VEGF is mediated through its high affinity receptors, which are selectively expressed on endothelial cells during embryogenesis and during tumor formation. Vascular endothelial growth factors regulate vascular development, angiogenesis and lymphangiogenesis by binding to a number of receptors. VEGFR-1 is required for the recruitment of haematopoietic stem cells and the migration of monocytes and macrophages, VEGFR-2 regulates vascular endothelial function and VEGFR-3 regulates lymphatic endothelial cell function.

Tyrosine kinase inhibitors have been developed such as carbozantinib (Exelixis Inc.) and pazopanib (GSK) and used as VEGFR inhibitors. Monoclonal antibodies have also been developed and used as VEGF inhibitors to block the binding of VEGF to its VEGFR to inhibit VEGF-induced signaling. For example WO 2006/055809 discloses monoclonal antibodies specific for VEGFR-1. US 2005/0123537 discloses antibodies that specifically inhibit VEGF binding to VEGFR-2; WO2017117384 discloses full length antibodies that bind specific domains of VEGFR-2.

With respect to VEGFR-2, therapeutic inhibition of the VEGFR-2 would be useful for the treatment of a number of diseases including cancers in order to inhibit or slow down or regress the growth of blood vessels to prevent or slow tumor growth.

There remains a need for agents that specifically inhibit VEGFR-2 receptor activity that have a desirable affinity and/or can overcome one or more disadvantages of currently known agents.

In order to address this concern, single domain antibodies specific for VEGFR-2 are now developed as an effective therapeutic agent. The anti-VEGFR-2 antibodies described herein may be useful, novel therapeutic antagonists for treatment of angiogenesis-associated diseases such as for cancer to prevent or slow down tumor growth.

### SUMMARY OF THE INVENTION

The present invention relates to anti-VEGFR-2 antibodies and uses thereof. More specifically, the antibodies are single domain antibodies (sdAbs) specific for VEGFR-2.

The present invention provides isolated or purified sdAbs or fragments and variants thereof specific for VEGFR-2 that bind to one or more epitopes thereof.

The single domain antibodies according to the invention are useful for inhibiting VEGFR-2-mediated signaling and for treating diseases and disorders caused by or related to VEGFR-2 activity and/or signaling.

Single domain antibodies (“sdAb” also called V_{HH} or nanobody) are part of a class of recombinant antibody fragments. Single-domain antibodies such as those identified herein are, in one or more aspects, known to possess stability under extreme temperature and pH; have superior tissue penetration ability due to their small size; can bind “hidden” epitopes; have a high solubility; and exhibit a rapid clearance in vivo.

The present invention provides isolated or purified antibodies or fragments and variants thereof specific for VEGFR-2, wherein the antibody or fragment or variant thereof binds to an epitope of VEGFR-2 as for example, but not limited to, epitopes described in U.S. Pat. No. 8,378,071 or WO 2017/117384 (the disclosures of which are incorporated by reference herein in its entirety).

The present invention further provides an isolated or purified sdAb or fragment or variant thereof, comprising a complementarity determining region of a CDR1; a CDR2; and a CDR3 wherein the sdAb antibody or fragment thereof is specific for VEGFR-2. The isolated or purified antibody or fragment thereof may be a single-domain antibody (sdAb) of any origin. For example, the sdAb may be of camelid origin (including members of the Camelidae family) or of human origin.

A sdAb comprises a single immunoglobulin domain that retains the immunoglobulin fold; most notably, only three CDR form the antigen-binding site. However, and as would be understood by those of skill in the art, not all CDR may be required for binding the antigen. For example, and without wishing to be limiting, one, two, or three of the CDR may contribute to binding and recognition of the antigen by the sdAb of the present invention. The CDR of the sdAb or variable domain are referred to herein as CDR1, CDR2, and CDR3, and numbered as defined by Kabat et al (1991b).

The isolated or purified antibody or fragment thereof of the invention may comprise one of the sequences of SEQ ID NO:2-30 with or without a linker sequence, in aspects the linker sequence may comprise a terminal cysteine, which in aspects is useful for chemical conjugation, or a sequence at least 85%, at least 86%, at least 87%, at least 88%, at least 89%, at least 90%, at least 91%, at least 92%, at least 93%, at least 94% or at least 95% identical thereto, or a sequence substantially identical thereto.

Linker sequences suitable for the single domain antibodies of the invention may be selected from the group consisting of SEQ ID NO:54-65. In aspects, the linker sequence may further comprise a C-terminal cysteine, for example as in SEQ ID NO:66-69. Sequences similar to these linker sequences may be used herein.

The isolated or purified antibodies or fragments thereof described herein may be in a multivalent display. For example, the isolated or purified antibodies or fragments thereof may be expressed linked to a Fc fragment; in one example, the Fc fragment may be a mouse Fc2b or human Fc1.

The present invention also provides a nucleic acid molecule encoding the isolated or purified antibodies or fragments thereof described above. Also encompassed by the present invention is a vector comprising the nucleic acid molecules described herein.

In aspects, the present invention provides an isolated polynucleotide comprising a nucleotide sequence selected from the group consisting of SEQ ID NO:31-53. The nucleotide sequence encodes an antibody or fragment thereof that specifically binds to VEGFR-2.

In aspects, the present invention provides an isolated polynucleotide comprising a nucleotide sequence that encodes an antibody or fragment thereof that specifically binds to VEGFR-2 and that is at least 80%, at least 85%, at least 86%, at least 87%, at least 88%, at least 89%, at least 90%, at least 91%, at least 92%, at least 93%, at least 94% or at least 95% homologous to the nucleotide sequence selected from the group consisting of any one of SEQ ID NO:31-53.

The present invention further provides the isolated or purified sdAb or fragments and variants thereof described herein immobilized onto a surface.

Additionally, the present invention provides the isolated or purified antibodies or fragments and variants thereof linked to a cargo molecule. The cargo molecule may be any suitable diagnostic or therapeutic agent known in the art.

The present invention also provides a method of directly blocking VEGFR-2 to help in reducing the ability of tumor cells to promote angiogenesis. The method comprises administering any one or more of the sdAbs of SEQ ID NO:2-30 or a functional fragment thereof, or a functional variant thereof with or without linker, with or without a terminal cysteine, or a combination thereof to a subject in need thereof.

The present invention further provides an in vivo method of detecting tumors expressing VEGFR-2, comprising: a) administering the isolated or purified sdAb or fragment thereof of the present invention linked to a diagnostic agent to a subject; and b) detecting the binding of the molecular imaging agent.

Diagnostic agents for use in the method may be radioisotope, a paramagnetic label, a fluorophore, a Near Infra-Red (NIR) fluorochrome or dye, an affinity label, or a detectable protein-based molecule via genetic fusion to the sdAb. Detection may be accomplished by any suitable imaging method including, but not limited to non-invasive optical imaging, ultrasound. MRI, PET, or SPECT.

As described herein, antibodies in the form of sdAb are presently described that are specific for VEGFR-2. The sdAb against VEGFR-2, which have an inhibitory effect on angiogenesis as described herein, are candidates for the development of antibody-based drugs against cancers expressing the receptor. In particular, in one aspect, SEQ ID NO:2, SEQ ID NO: 19 and SEQ ID NO:25 recognize an overlapping epitope on VEGFR-2.

The sdAb disclosed herein may block the VEGFR-2 leading to decreased angiogenesis in the tumor. Advantageously, these antibodies may be more specific for tumors that express/over-express VEGFR-2 than chemotherapeutic agents.

In an aspect, the present invention provides a method of inhibiting angiogenesis or reducing/regressing tumor growth by administering a therapeutically effective amount of an antibody or fragment thereof that specifically bind to VEGFR-2 and comprises any one of SEQ ID NO:2-53. In an aspect, the present invention provides a method of decreasing angiogenesis in a tumor by administering a therapeutically effective amount of an antibody or fragment thereof that specifically bind to VEGFR-2 and comprises any one of sdAbs of SEQ ID NO:2-53.

In aspects, the present invention is directed to compositions comprising one or more of the antibodies of the present invention and any fragments and variants thereof. The compositions may comprise pharmaceutically acceptable excipients and the like and optionally other therapeutic agents.

Further aspects of the invention are as follows:

According to an aspect of the invention is a sdAb that binds to VEGFR-2.

According to an aspect of the invention is a polypeptide comprising the sequence of any one of SEQ ID NO:2-30 or a fragment or variant thereof.

According to an aspect of the invention is a polypeptide consisting of the sequence of any one of SEQ ID NO:2-30 or a fragment or variant thereof.

According to an aspect of the invention is a polypeptide of the invention that binds to VEGFR-2.

According to an aspect of the invention, the polypeptide of the invention is a single domain antibody.

According to an aspect of the invention, the fragments or variants have at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NO:2-30.

According to an aspect of the invention, the fragments or variants of any one of the polypeptides of SEQ ID NO:2-30 are functional and bind to VEGFR-2.

According to an aspect of the invention is a composition comprising the polypeptide, fragment, or variant of any one of SEQ ID NO:2-30 optionally comprising a pharmaceutically acceptable carrier and/or a therapeutic agent.

According to an aspect of the invention is a polypeptide comprising the sequence of SEQ ID NO:2 or fragments or variants thereof having more than 93% identity to SEQ ID NO:2, or fragments or variants thereof having more than 85% identity to SEQ ID NO:2, wherein the fragments or variants thereof comprise more than 116 amino acid residues.

According to an aspect of the invention is a polypeptide comprising the sequence of SEQ ID NO: 11 or fragments or variants thereof having more than 77% identity to SEQ ID NO:11.

According to an aspect of the invention is a polypeptide comprising the sequence of SEQ ID NO: 19 or fragments or variants thereof having more than 88% identity to SEQ ID NO:19.

According to an aspect of the invention is a polypeptide comprising the sequence of SEQ ID NO:6 or fragments or variants thereof having more than 86% identity to SEQ ID NO:6.

According to an aspect of the invention is a polypeptide comprising the sequence of SEQ ID NO:25 or fragments or variants thereof having more than 80% identity to SEQ ID NO:25.

According to an aspect of the invention is a polypeptide comprising the sequence of SEQ ID NO:26 or fragments or variants thereof having more than 80% identity to SEQ ID NO:26.

According to an aspect of the invention is a polypeptide comprising the sequence of SEQ ID NO:30 or fragments or variants thereof having more than 80% identity to SEQ ID NO:30.

According to an aspect of the invention is a polypeptide comprising the sequence of SEQ ID NO:8 or fragments or variants thereof having more than 80% identity to SEQ ID NO:8.

According to an aspect of the invention is a polypeptide comprising the sequence of SEQ ID NO: 10 or fragments or variants thereof having more than 80% identity to SEQ ID NO:10.

According to an aspect of the invention is a polypeptide comprising the sequence of SEQ ID NO:15 or fragments or variants thereof having more than 80% identity to SEQ ID NO:15.

According to an aspect of the invention is a polypeptide comprising the sequence of SEQ ID NO:16 or fragments or variants thereof having more than 80% identity to SEQ ID NO:16.

According to an aspect of the invention is a polypeptide comprising the sequence of SEQ ID NO: 17 or fragments or variants thereof having more than 80% identity to SEQ ID NO:17.

According to an aspect of the invention is a polypeptide comprising the sequence of SEQ ID NO:22 or fragments or variants thereof having more than 80% identity to SEQ ID NO:22.

According to an aspect of the invention is a polypeptide of the invention further comprising a linker sequence.

According to an aspect of the invention, the linker sequence comprises a terminal cysteine.

According to an aspect of the invention, the polypeptide of the invention comprises a linker sequence selected from the group consisting of SEQ ID NO:54-69.

According to an aspect of the invention is a polypeptide comprising the sequence of SEQ ID NO:3-7, 9, 12-14, 16, 18, 20, 21, 23, 24, 26, 27, 29, or 30 and further comprising a linker sequence, in aspects selected from the group consisting of SEQ ID NO:54-69.

According to an aspect of the invention are fragments or variants having more than 95%, 96%, 97%, 98%, or 99% identity to SEQ ID NO:3-7, 9, 12-14, 16, 18, 20, 21, 23, 24, 26, 27, 29, or 30.

According to an aspect of the invention are fragments or variants having more than 95%, 96%, 97%, 98%, or 99% identity to SEQ ID NO:3-7, 9, 12-14, 16, 18, 20, 21, 23, 24, 26, 27, 29, or 30.

According to an aspect of the invention is the sequence of SEQ ID NO:2-30.

According to an aspect of the invention is the sequence of SEQ ID NO:2-30.

According to an aspect of the invention, the polypeptide of the invention binds to an epitope of VEGFR-2.

According to an aspect of the invention, fragments and variants of the polypeptide of the invention bind to an epitope of VEGFR-2.

According to an aspect of the invention, the polypeptide of the invention is coupled to a fusion partner sequence.

In aspects of the invention, the fusion partner sequence comprises the sequence of SEQ ID NO:71 or a variant thereof.

In aspects of the invention the fusion partner sequence consists of the sequence of SEQ ID NO:71.

According to an aspect of the invention is an antibody or fragment thereof comprising a polypeptide comprising the sequence of any one of SEQ ID NO:2-30. In aspects, the antibody or fragment thereof comprises at least one CDR having a sequence selected from the group consisting of SYAMG, AISWSDDSTYYANSVKG. HKSLQRPDEYTY and a sequence at least 70%, at least 75%, at least 80%, at least 85%, at least 90% or at least 90% identical thereto which binds VEGFR2. In further aspects, the antibody or fragment is a single domain antibody.

In all aspects, the antibody or fragment or variant thereof of the invention specifically binds to VEGFR-2.

In aspects of the invention, the antibody or fragment specifically binds to a complex of VEGF and VEGFR-2.

In aspects of the invention, the antibody or fragment binds with a K_{D} of less than 10⁻⁷ M.

In aspects of the invention, the antibody or fragment is humanized.

In aspects of the invention, the antibody or fragment is conjugated to another moiety.

In aspects of the invention, the antibody or fragment is in a multivalent display.

In aspects of the invention, the antibody is linked to an Fc fragment.

In aspects of the invention, the Fc fragment is mouse Fc2b or human Fc1.

In aspects of the invention, the antibody or fragment is linked to a cargo molecule.

In aspects of the invention, the cargo molecule is a therapeutic molecule.

In aspects of the invention, the cargo molecule is a diagnostic agent.

In aspects of the invention, the antibody or fragment comprises a flag tag sequence.

In aspects of the invention, the antibody or fragment thereof is of dromedary, camel, llama, alpaca origin.

According to an aspect of the invention is a nucleic acid molecule encoding the polypeptide or the antibody or the fragments/variants thereof.

According to an aspect of the invention is a nucleic acid molecule comprising a sequence selected from SEQ ID NO:31-53.

According to an aspect of the invention is an expression vector comprising any of the nucleic acid molecules disclosed herein and selected from SEQ ID NO:31-53.

According to an aspect of the invention is a recombinant host cell comprising the expression vector comprising any of the nucleic acid molecules disclosed herein.

According to an aspect of the invention is a recombinant host cell expressing, displaying, and/or secreting the polypeptide of the invention and/or the antibody of the invention.

According to an aspect of the invention is a composition comprising one or more of the polypeptides of the invention and/or one or more of the antibodies of the invention.

According to an aspect of the invention is a method for reducing and/or preventing angiogenesis, the method comprising administering the polypeptide of any one of SEQ ID NO:2-30, and/or the antibody and/or the compositions comprising such to a subject in need thereof.

According to an aspect of the invention is an in vivo method of detecting VEGFR-2-expressing tumors, comprising: a) administering the single domain antibody of the invention comprising a polypeptide of SEQ ID NO:2-30 or fragment thereof to a subject; and b) detecting the binding of the single domain antibody.

According to an aspect of the invention, is a method of producing a single domain antibody or fragment thereof comprising culturing the cell comprising a nucleic acid sequence encoding the polypeptide of any one of SEQ ID NO:2-30 or fragment or variant thereof, under conditions permitting expression of the antibody or fragment thereof.

According to an aspect of the invention, is a method of modulating activity of VEGFR-2 in a mammal comprising administering to the mammal an effective amount of an antibody or fragment thereof comprising a sequence selected from any one of SEQ ID NO:2-30.

According to an aspect of the invention is a method of inhibiting/reducing angiogenesis in a mammal comprising administering to the mammal an effective amount of an antibody or fragment thereof of the invention comprising a polypeptide sequence of any one of SEQ ID NO:2-30. In aspects, the angiogenesis is in a tumor in said mammal.

According to an aspect of the invention is a method of reducing tumor growth in a mammal comprising administering to the mammal an effective amount of an antibody or fragment thereof comprising a polypeptide sequence of any one of SEQ ID NO:2-30.

According to an aspect of the invention is a single domain antibody that binds to VEGFR-2.

According to an aspect of the invention is a camelid single domain antibody that binds to VEGFR-2.

According to an aspect of the invention is a human/humazined single domain antibody that binds to VEGFR-2.

According to an aspect of the invention is a synthetic single domain antibody that binds to VEGFR-2.

According to an aspect of the invention is a kit for detecting VEGFR-2 in a biological sample, such as a blood sample or tissue sample. For example, to confirm a cancer diagnosis in a subject, a biopsy can be performed to obtain a tissue sample for histological examination.

Alternatively, a blood sample can be obtained to detect the presence of VEGFR-2 protein or fragment. Kits for detecting a polypeptide will typically comprise an antibody as described herein comprising any one or more of SEQ ID NO:2-30 that specifically binds VEGFR-2 or a nucleic acid encoding any one of SEQ ID NO:2-30. In a further embodiment, the antibody is labeled (for example, with a fluorescent, radioactive, or an enzymatic label). In a further aspect, a kit includes instructional materials disclosing means of use of the antibodies that bind VEGFR-2. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In an aspect, the diagnostic kit comprises an immunoassay. Although the details of the immunoassays may vary with the particular format employed, the method of detecting VEGFR-2 in a biological sample generally includes the steps of contacting the biological sample with an antibody which specifically reacts, under immunologically reactive conditions, to VEGFR-2. The antibody is allowed to specifically bind under immunologically reactive conditions to form an immune complex, and the presence of the immune complex (bound antibody) is detected directly or indirectly.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following detailed description of typical aspects described herein will be better understood when read in conjunction with the appended drawings. For the purpose of illustrating the invention, there are shown in the drawings aspects which are presently typical. It should be understood, however, that the invention is not limited to the precise arrangements and instrumentalities of the aspects shown in the drawings.

FIG. 1 shows size exclusion column chromatograms for AB1 (SEQ ID NO:2), AB2 (SEQ ID NO: 11), AB3 (SEQ ID NO:19), and AB4 (SEQ ID NO:25).

FIG. 2 shows binding of AB1 (SEQ ID NO:2), AB2 (SEQ ID NO:13), AB3 (SEQ ID NO:21), and AB4 (SEQ ID NO:27) to human VEGFR-2/Fc.

FIG. 3 shows binding kinetics for AB1 (SEQ ID NO:7) binding to human VEGFR-2/Fc.

FIG. 4 shows (a) epitope mapping of the single domain anti-VEGFR-2 antibodies of the present invention to VEGFR-2 and (b) overlapping binding of epitopes for AB1 (SEQ ID NO:2), AB2 (SEQ ID NO:13), AB3 (SEQ ID NO:23), and AB4 (SEQ ID NO:27).

FIG. 5 shows antibody binding and cross-reactivity of AB1 m (SEQ ID NO:9), AB2 (SEQ ID NO:13), AB3m (SEQ ID NO:23), and AB4 (SEQ ID NO:27) to VEGFR-1, VEGFR-2 and VEGFR-3. All four single domain antibodies were used to make urease (“DOS47”) conjugates. These conjugates were tested by ELISA for their ability to bind the antigen VEGFR-2 and also their ability to cross-react with VEGFR-1 and VEGFR-3. All four antibody conjugates bind to recombinant VEGFR2/Fc, with the strongest binding observed with the llama antibody conjugates (consistent with K_{D} values determined in FIG. 3). All antibodies show some cross-reactivity to VEGFR1/Fc. There was no detectable binding by any of the antibodies to VEGFR3/Fc.

FIG. 6 shows the results of VEGF competition assays for AB1 (SEQ ID NO:2), AB2 (SEQ ID NO:13), AB3 (SEQ ID NO:23), and AB4 (SEQ ID NO:27). This was done to assess whether the antibodies recognize a region near the VEGF binding pocket. Antibody-urease conjugates were mixed with VEGF at a variety of different molar ratios, and then tested for binding to VEGFR2/Fc captured on ELISA plates. The binding of the two human antibody conjugates (AB2—(SEQ ID NO:13) & AB3—(SEQ ID NO:21) DOS47) to VEGFR2 was inhibited by VEGF, suggesting these antibodies and VEGF bind to overlapping sites. The binding of AB1-DOS47 was only minimally affected by VEGF, suggesting that the AB1 antibody and VEGF bind to distinct sites. Interestingly, the binding of AB4-DOS47 to VEGFR2 was enhanced by the presence of VEGF, suggesting that the AB4 antibody binds better to the VEGF/VEGFR2 complex than to VEGFR2 alone.

FIG. 7 shows AB1 (SEQ ID NO:9)-DOS47 (A) and AB3 (SEQ ID NO:23)-DOS47 (B) antibody-urease conjugates mixed with each of the four uncoupled antibodies (SEQ ID NO:7, 13, 21, and 27)(or anti-CEACAM6 as a negative control) at a variety of different molar ratios, and then tested for binding to VEGFR2/Fc coated on ELISA plates. Binding of each antibody-urease conjugate was inhibited by the corresponding uncoupled antibody. In addition, the AB3-urease conjugate was inhibited by uncoupled AB2 antibody, suggesting that the two human antibodies share at least partially overlapping epitopes. The uncoupled AB3 antibody also partially inhibited the binding of AB1-DOS47, although only at very high molar ratios.

FIG. 8 shows binding of antibodies and antibody-urease conjugates to 293/KDR cells, which are HEK293 cells that have been transfected to stably express VEGFR2 (KDR). 293/KDR cells were stained with antibodies or antibody-urease conjugates and binding was detected by flow cytometry. Antibodies AB1 (SEQ ID NO:6) and AB2 (SEQ ID NO: 18) bind to VEGFR2 expressed on 293/KDR cells.

FIG. 9 shows a deconvoluted mass spectrum of the V21H1 (SEQ ID NO:3) antibody after activation by cross-linker and linkage to cysteine showing the distribution of non-activated antibody, antibody activated by one cross-linker and antibody activated by two cross-linkers.

FIG. 10 shows RP-HPLC chromatograms of V21H4 (SEQ ID NO:6) samples at different refolding time points. Blue line: sample at refolding time 0, immediately after the SP pooled fraction was mixed with refolding buffer. Red line: refolding time point 2 hours after mixing. Green line: refolding sample 4 hours after time 0 and 2 hours after addition of 1.2 mM cystamine. Unfolded antibody elutes at 12.513 min and folded antibody elutes at 10.958 min.

FIG. 11: (A-C) Screen snapshots of intact protein mass spectra of V21H4 (SEQ ID NO:6) samples from BiopharmaLynx. (A) Deconvoluted spectrum of V21H4 (SEQ ID NO:6) showing the attachment of a half-cystamine to the C-terminal cysteine by forming a disulfide bond during refolding. (B) The deconvoluted spectrum of V21H4 after reduction with 2 mM TCEP showing the detachment of the C-terminal half-cystamine. (C) The deconvoluted spectrum of the reduced V21H4 after alkylation with iodoacteamide showing the C-terminal cysteine is accessible to a sulfhydryl activation cross-linker. (D) Deconvoluted mass spectrum of V21H4 after activation by cross-linker and linkage to cysteine. V21H4 antibody activated by BM(PEG)₂ generates a single activated species.

FIG. 12: (A) SDS-PAGE of V21H1-(SEQ ID NO:3) DOS47 and V21H4-(SEQ ID NO:6) DOS47. Bands labelled in red with 1, 2 or 3 are cluster numbers. Lane 1: molecular weight ladder. Lane 2: HPU. Lanes 3 and 4: V21H1-DOS47. Lanes 5 and 6: V21H4-DOS47. (B) Size exclusion chromatograms of V21H1, V21H4, high purity urease (HPU), V21H1-DOS47 and V21H4-DOS47.

FIG. 13: (A) ELISA of biotin-V21H4 (SEQ ID NO:6) (black), V21H1-DOS47 (SEQ ID NO:3) (green) and V21H4-(SEQ ID NO:6) DOS47 (red) binding to recombinant VEGFR2/Fc. Results shown are representative of 2-5 experiments performed for each sample and are presented as the means and SE of samples tested in triplicate. (B) Binding of biotin-V21H4 (black) and V21H4-DOS47 (red) to VEGFR2 expressed by 293/KDR cells. Binding was quantified by flow cytometry. Results shown are representative of 2-3 experiments performed for each sample and are presented as the means and SE of samples tested in duplicate. (C) Urease enzyme activity of V21H4-DOS47 at different antibody/urease conjugation ratios. The dotted line represents unconjugated urease activity. (D) ELISA of V21H4-DOS47 with different antibody-urease conjugation ratios binding to recombinant VEGFR2/Fc. Results shown are representative of two experiments performed for each sample and are presented as the means and SE of samples tested in duplicate.

FIG. 14: Western blot of V21H4 (SEQ ID NO:6), HPU, and V21H4-(SEQ ID NO:6) DOS47. Blots were probed with (A) an anti-llama antibody or (B) an anti-urease antibody. Lane MW: molecular weight ladder. Lane 1: V21H4. Lane 2: HPU. Lanes 3 and 4: V21H4-DOS47.

FIG. 15: (A) Screen snapshots of raw LC-MS (TIC) chromatograms of tryptic digests of HP urease (top) and V21H4-(SEQ ID NO:6) DOS47 (bottom) samples processed by BiopharmaLynx software. (B) Screen snapshots of b/y fragment profiles of conjugation site UC₈₂₄-VC₁₃₆ mapped as the V21H4 peptide GGGEEDDGC (top) modified by UC₈₂₄-BM(PEG)₂ and as the urease peptide LLCVSEATTVPLS (bottom) modified by VC₁₃₆-BM(PEG)₂.

### DETAILED DESCRIPTION OF THE INVENTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting.

In understanding the scope of the present application, the articles “a”, “an”, “the”, and “said” are intended to mean that there are one or more of the elements.

Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms may refer to a measurable value such as an amount, a temporal duration, and the like, is meant to encompass variations of ±20% or ±10%, more typically ±5%, even more typically ±1%, and still more typically ±0.1% from the specified value, as such variations are appropriate to perform the disclosed methods.

“Activation”, as used herein, refers to the state of an immune cell, such as a CIK cell or T cell, that has been sufficiently stimulated to induce detectable cellular proliferation. Activation can also be associated with induced cytokine production, and detectable effector functions. The term “activated T cells” refers to, among other things, T cells that are undergoing cell division.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The term “antibody”, also referred to in the art as “immunoglobulin” (Ig), used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (CH, CH2, CH3) domains. Interaction of the heavy and light chain variable domains (VH and VL) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen. Various schemes exist for identification of the regions of hypervariability, the two most common being those of Kabat and of Chothia and Lesk. Kabat et al (1991a; 1991b) define the “complementarity-determining regions” (CDR) based on sequence variability at the antigen-binding regions of the VH and VL domains. Chothia and Lesk (1987) define the “hypervariable loops” (H or L) based on the location of the structural loop regions in the VH and VL domains. As these individual schemes define CDR and hypervariable loop regions that are adjacent or overlapping, those of skill in the antibody art often utilize the terms “CDR” and “hypervariable loop” interchangeably, and they may be so used herein. For this reason, the regions forming the antigen-binding site are referred to as CDR L1, CDR L2, CDR L3, CDR H1, CDR H2, CDR H3 in the case of antibodies comprising a VH and a VL domain; or as CDR1, CDR2, CDR3 in the case of the antigen-binding regions of either a heavy chain or a light chain. The CDR/loops are referred to herein according to the IMGT numbering system (Lefranc et al., 2003), which was developed to facilitate comparison of variable domains. In this system, conserved amino acids (such as Cys23, Trp41, Cys 104, Phe/Trp 118, and a hydrophobic residue at position 89) always have the same position. Additionally, a standardized delimitation of the framework regions (FR1: positions 1 to 26; FR2: 39 to 55; FR3: 66 to 104; and FR4: 118 to 128) and of the CDR (CDR1: 27 to 38, CDR2: 56 to 65; and CDR3: 105 to 117) is provided.

An “antibody fragment” as generally referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of VL and VH connected with a peptide linker), Fab, F(ab′)2, single domain antibody (sdAb; a fragment composed of a single VL or VH), and multivalent presentations of any of these.

Antibody fragments of any one of SEQ ID NO:2-30 are those understood by one of skill in the art to retain biological activity to bind to VEGFR-2.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In a non-limiting example, the antibody fragment may be an sdAb derived from naturally-occurring sources. Heavy chain antibodies of camelid origin (Hamers-Casterman et al, 1993) lack light chains and thus their antigen binding sites consist of one domain, termed V_{HH}, sdAb have also been observed in shark and are termed V_{NAR} (Nuttall et al, 2003). Other sdAb may be engineered based on human Ig heavy and light chain sequences (Jespers et al, 2004: To et al, 2005). As used herein, the term “sdAb” includes those sdAb directly isolated from V_{H}, V_{HH}, V_{L}, or V_{NAR} reservoir of any origin through phage display or other technologies, sdAb derived from the aforementioned sdAb, recombinantly produced sdAb, as well as those sdAb generated through further modification of such sdAb by humanization, affinity maturation, stabilization, solubilization, e.g., camelization, or other methods of antibody engineering. Also encompassed by the present invention are homologues, derivatives, or fragments that retain the antigen-binding function and specificity of the sdAb.

SdAbs have high thermostability, high detergent resistance, relatively high resistance to proteases (Dumoulin et al, 2002) and high production yield (Arbabi-Ghahroudi et al, 1997); they can also be engineered to have very high affinity by isolation from an immune library (Li et al, 2009) or by in vitro affinity maturation (Davies & Riechmann, 1996).

A person of skill in the art would be well-acquainted with the structure of a single-domain antibody (see, for example, 3DWT, 2P42 in Protein Data Bank). A sdAb comprises a single immunoglobulin domain that retains the immunoglobulin fold; most notably, only three CDR form the antigen-binding site. However, and as would be understood by those of skill in the art, not all CDR may be required for binding the antigen. For example, and without wishing to be limiting, one, two, or three of the CDR may contribute to binding and recognition of the antigen by the sdAb of the present invention. The CDR of the sdAb or variable domain are referred to herein as CDR1, CDR2, and CDR3, and numbered as defined by Kabat et al (1991b).

Epitope: An antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or 8 to 10 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996). In one embodiment, an epitope binds an MHC molecule, such an HLA molecule or a DR molecule. These molecules bind polypeptides having the correct anchor amino acids separated by about eight to about ten amino acids, such as nine amino acids.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a tumor sample, a cell or a biological fluid.

The term “anti-tumor effect” or “treatment of cancer” as used herein, refers to a biological effect which can be manifested by a decrease in tumor volume, a decrease in the number of tumor cells, a decrease in the rate of tumor growth, a decrease in the number of metastases, stabilized disease, an increase in life expectancy, or amelioration of various physiological symptoms associated with the cancerous condition. An “anti-tumor effect” can also be manifested by the ability of the peptides, polynucleotides, cells and antibodies described herein in prevention of the occurrence of tumor in the first place.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from a different species.

“Syngeneic” refers to a graft derived from an identical individual.

“Co-stimulatory ligand” as the term is used herein, includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e g, naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared.times.100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine. “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “transposon” or “transposable element” is a DNA sequence that can change its position within a genome, sometimes creating or reversing mutations and altering the cell's genome size. Transposition often results in duplication of the transposon. There are two distinct types of transposon: class II transposons, which consist of DNA that moves directly from place to place; and class I transposons, which are retrotransposons that first transcribe the DNA into RNA and then use reverse transcriptase to make a DNA copy of the RNA to insert in a new location. Transposons typically interact with a transposase, which mediates the movement of the transposon. Non-limiting examples of transposon/transposase systems include Sleeping Beauty, Piggybac, Frog Prince, and Prince Charming.

By the term “modulating.” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding.” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a super agonist anti-CD28 antibody, and a super agonist anti-CD2 antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

As used herein, “treatment” or “therapy” is an approach for obtaining beneficial or desired clinical results. For the purposes described herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as cancer, or may render the cells more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to treat cancer. Effective amounts of the compounds described herein may vary according to factors such as the disease state, age, sex, and weight of the subject. Dosage or treatment regimes may be adjusted to provide the optimum therapeutic response, as is understood by a skilled person. For example, administration of a therapeutically effective amount of an anti-VEGFR-2 sdAb is, in aspects, sufficient to reduce, inhibit or prevent formation of blood vessels associated with tumor progression or metastasis.

Moreover, a treatment regime of a subject with a therapeutically effective amount may consist of a single administration, or alternatively comprise a series of applications. The length of the treatment period depends on a variety of factors, such as the severity of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The antibodies described herein may, in aspects, be administered before, during or after treatment with conventional therapies for the disease or disorder in question, such as cancer.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The terms “patient,” “subject.” “individual,” and the like are used interchangeably herein, and refer to any animal, or cells thereof whether in vitro or in situ, amenable to the methods described herein.

Moreover, the terms “patient”, “subject” and “individual” includes living organisms in which an immune response can be elicited (e.g., mammals). In certain non-limiting aspects, the patient, subject or individual is a mammal and includes humans, dogs, cats, mice, rats, and transgenic species thereof. The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

Isolated: An “isolated” biological component (such as a protein) has been substantially separated or purified away from other biological components in the cell of the organism in which the component naturally occurs, i.e., chromosomal and extra-chromosomal DNA and RNA, other proteins and organelles. Proteins and peptides that have been “isolated” include proteins and peptides purified by standard purification methods. The term also includes proteins and peptides prepared by recombinant expression in a host cell, as well as chemically synthesized proteins and peptides.

“Tumour”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. As used herein, cancer or cancerous is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The cancer to be treated may be any type of malignancy and, in an aspect, is lung cancer, including small cell lung cancer and non-small cell lung cancer (e.g. adenocarcinoma), pancreatic cancer, colon cancer (e.g. colorectal carcinoma, such as, for example, colon adenocarcinoma and colon adenoma), oesophageal cancer, oral squamous carcinoma, tongue carcinoma, gastric carcinoma, liver cancer, nasopharyngeal cancer, hematopoietic tumours of lymphoid lineage (e.g. acute lymphocytic leukemia, B-cell lymphoma, Burkitt's lymphoma), non-Hodgkin's lymphoma (e.g. mantle cell lymphoma), Hodgkin's disease, myeloid leukemia (for example, acute myelogenous leukemia (AML) or chronic myelogenous leukemia (CML)), acute lymphoblastic leukemia, chronic lymphocytic leukemia (CLL), thyroid follicular cancer, myelodysplastic syndrome (MDS), tumours of mesenchymal origin, soft tissue sarcoma, liposarcoma, gastrointestinal stromal sarcoma, malignant peripheral nerve sheath tumour (MPNST), Ewing sarcoma, leiomyosarcoma, mesenchymal chondrosarcoma, lymphosarcoma, fibrosarcoma, rhabdomyosarcoma, melanoma, teratocarcinoma, neuroblastoma, brain tumours, medulloblastoma, glioma, benign tumour of the skin (e.g. keratoacanthoma), breast carcinoma (e.g. advanced breast cancer), kidney carcinoma, nephroblastoma, ovary carcinoma, cervical carcinoma, endometrial carcinoma, bladder carcinoma, prostate cancer, including advanced disease and hormone refractory prostate cancer, testicular cancer, osteosarcoma, head and neck cancer, epidermal carcinoma, multiple myeloma (e.g. refractory multiple myeloma), or mesothelioma. In an aspect, the cancer cells are derived from a solid tumour. Typically, the cancer cells are derived from a breast cancer, colorectal cancer, melanoma, ovarian cancer, pancreatic cancer, gastric cancer, lung cancer, or prostate cancer. More typically, the cancer cells are derived from a prostate cancer, a lung cancer, a breast cancer, or a melanoma.

A “chemotherapeutic agent” is a chemical compound useful in the treatment of cancer. Examples of chemotherapeutic agents include alkylating agents such as thiotepa, CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins such as bullatacin and bullatacinone; camptothecins such as topotecan; bryostatin: callystatin; CC-1065 and its adozelesin, carzelesin and bizelesin synthetic analogues; cryptophycins such as cryptophycin 1 and cryptophycin 8; dolastatin; duocarmycins such as the synthetic analogues KW-2189 and CB1-TM1; eleutherobin; pancratistatin; sarcodictyins; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics, for example calicheamicin, especially calicheamicin gamma1I and calicheamicin omegaI1, dynemicin, including dynemicin A, bisphosphonates, such as clodronate, esperamicins, neocarzinostatin chromophore and related chromoprotein enediyne antibiotic chromophores; aclacinomysins; actinomycin; authramycin; azaserine; bleomycins; cactinomycin; carabicin; carminomycin; carzinophilin; chromomycins; dactinomycin; daunorubicin; detorubicin; 6-diazo-5-oxo-L-norleucine; ADRIAMYCIN™ doxorubicin, including morpholino-doxorubicin, cyanomorpholino-doxorubicin, 2-pyrrolino-doxorubicin and deoxydoxorubicin; epirubicin; esorubicin; idarubicin; marcellomycin; mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; epothilones; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKTM polysaccharide complex; razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethan; vindesine; dacarbazine; mannomustine; mitobronitol; mitolactol; pipobroman; gacytosine; arabinoside (“Ara-C”); taxoids, such as TAXOL™ paclitaxel, ABRAXANE™ Cremophor-free, albumin-engineered nanoparticle formulation of paclitaxel, TAXOTERE™ and doxetaxel; chloranbucil; GEMZAR™ gemcitabine; 6-thioguanine; mercaptopurine; methotrexate; platinum coordination complexes such as cisplatin, oxaliplatin and carboplatin; vinblastine; platinum; etoposide (VP-16); ifosfamide; vincristine; NAVELBINE™ vinorelbine; novantrone; teniposide; edatrexate; daunomycin; aminopterin; xeloda; ibandronate; irinotecans such as CPT-11; topoisomerase inhibitors such as RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumours such as anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestane, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signalling pathways implicated in aberrant cell proliferation, such as, for example, PKC-alpha, Ralf and H-Ras; ribozymes such as a VEGF expression inhibitor (e.g., ANGIOZYME™ ribozyme) and a HER2 expression inhibitor; antibodies such as an anti-VEGF antibody (e.g., AVASTIN™ antibody); vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; and pharmaceutically acceptable salts, acids or derivatives of any of the above.

In aspects, the antibodies described herein act additively or synergistically with other conventional anti-cancer treatments.

“Variants” are biologically active antibodies or fragments thereof having an amino acid sequence that differs from the sequence of an anti-VEGFR-2 sdAb, such as those set out in SEQ ID NO:2-30, by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain VEGFR-2 binding ability. Variants also include polypeptides wherein one or more amino acid residues are added at the N- or C-terminus of, or within, the comparative sequence. For example “MQV” at the N-terminal end can be substituted with “MKKQV” and still retain binding activity to VEGFR-2. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the sdAbs described herein, wherein “biological” activity refers to a biological function (either inhibitory or stimulatory) caused by a the sdAbs.

Thus, “biologically active” or “biological activity” when used in conjunction with “anti-VEGFR-2 sdAbs” means an anti-VEGFR-2 sdAb or fragment thereof that exhibits or shares an effector function of anti-VEGFR-2 antibodies. One biological activity of such an antibody is its ability to inhibit, at least in part, vascular formation.

The terms “inhibit” or “inhibitory” mean that a function or activity of VEGFR-2 is decreased, limited, blocked, or neutralized. These terms encompass a complete or partial inhibition in VEGFR-2 function or activity.

As used herein, an “anti-VEGFR-2 single domain antibody” includes modifications of an anti-VEGFR-2 antibody of the present invention that retains specificity for VEGFR-2. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). Modifications further include, but are not limited to conjugation to detectable reporter moieties. Modifications that extend antibody half-life (e.g., pegylation) are also included. Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein, for the conjugation of doxorubicin and those described by Amon et al., Adv. Exp. Med. Biol. 303, 79-90 (1991) and by Kiseleva et al, Mol. Biol. (USSR)25, 508-514 (1991), both of which are incorporated by reference herein.

The antibody or fragment thereof of the present invention is specific for VEGFR-2 whose expression is elevated in many solid tumors such as but not limited to breast, pancreatic, ovarian, lung and colon cancer.

The sequence of VEGFR-2 (also known as KDR D1-7, sKDR D1-7, Kinase insert domain receptor, Protein-tyrosine kinase receptor Flk-1, CD309, type I receptor tyrosine kinase, FLK1) is known and may be as that illustrated in U.S. 2009/0247467 showing human and murine sequences (the disclosure of which is incorporated herein in its entirety). In aspects the protein sequence of VEGFR-3 may be, but is not limited to the sequence of SEQ ID NO:1:

MQSKVLLAVA LWLCVETRAA SVGLPSVSLD LPRLSIQKDI
LTIKANTTLQ ITCRGQRDLD WLWPNNQSGS EQRVEVTECS
DGLFCKTLTI PKVIGNDTGA YKCFYRETDL ASVIYVYVQD
YRSPFIASVS DQHGVVYITE NKNKTVVIPC LGSISNLNVS
LCARYPEKRF VPDGNRISWD SKKGFTIPSY MISYAGMVFC
EAKINDESYQ SIMYIVVVVG YRIYDVVLSP SHGIELSVGE
KLVLNCTART ELNVGIDFNW EYPSSKHQHK KLVNRDLKTQ
SGSEMKKFLS TLTIDGVTRS DQGLYTCAAS SGLMTKKNST
FVRVHEKPFV AFGSGMESLV EATVGERVRI PAKYLGYPPP
EIKWYKNGIP LESNHTIKAG HVLTIMEVSE RDTGNYTVIL
TNPISKEKQS HVVSLVVYVP PQIGEKSLIS PVDSYQYGTT
QTLTCTVYAI PPPHHIHWYW QLEEECANEP SQAVSVTNPY
PCEEWRSVED FQGGNKIEVN KNQFALIEGK NKTVSTLVIQ
AANVSALYKC EAVNKVGRGE RVISFHVTRG PEITLQPDMQ
PTEQESVSLW CTADRSTFEN LTWYKLGPQP LPIHVGELPT
PVCKNLDTLW KLNATMFSNS TNDILIMELK NASLQDQGDY
VCLAQDRKTK KRHCVVRQLT VLERVAPTIT GNLENQTTSI
GESIEVSCTA SGNPPPQIMW FKDNETLVED SGIVLKDGNR
NLTIRRVRKE DEGLYTCQAC SVLGCAKVEA FFIIEGAQEK
TNLEIIILVG TAVIAMFFWL LLVIILRTVK RANGGELKTG
YLSIVMDPDE LPLDEHCERL PYDASKWEFP RDRLKLGKPL
GRGAFGQVIE ADAFGIDKTA TCRTVAVKML KEGATHSEHR
ALMSELKILI HIGHHLNVVN LLGACTKPGG PLMVIVEFCK
FGNLSTYLRS KRNEFVPYKT KGARFRQGKD YVGAIPVDLK
RRLDSITSSQ SSASSGFVEE KSLSDVEEEE APEDLYKDFL
TLEHLICYSF QVAKGMEFLA SRKCIHRDLA ARNILLSEKN
VVKICDFGLA RDIYKDPDYV RKGDARLPLK WMAPETIFDR
VYTIQSDVVS FGVLLWEIFS LGASPYPGVK IDEEFCRRLK
EGTRMRAPDY TTPEMYQTML DCWHGEPSQR PTFSELVEHL
GNLLQANAQQ DGKDYIVLPI SETLSMEEDS GLSLPTSPVS
CMEEEEVCDP KFHYDNTAGI SQYLQNSKRK SRPVSVKTFE
DIPLEEPEVK VIPDDNQTDS GMVLASEELK TLEDRTKLSP
SFGGMVPSKS RESVASEGSN QTSGYQSGYH SDDTDTTVYS
SEEAELLKLI EIGVQTGSTA QILQPDSGTT LSSPPV.

Ranges: throughout this disclosure, various aspects described herein can be presented in a range format. It should be understood that the description in range format is merely for convenience and brevity and should not be construed as an inflexible limitation on the scope described herein. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Many patent applications, patents, and publications are referred to herein to assist in understanding the aspects described. Each of these references are incorporated herein by reference in their entirety.

The present invention further provides an isolated or purified single domain antibody or fragment thereof, comprising a complementarity determining region CDR1; a CDR2; and a CDR3 wherein the antibody or fragment thereof is specific for VEGFR-2. One or more of the CDR's may bind the VEGFR-2. The antibody as just described may recognize and bind to an epitope of the amino acid sequence of VEGFR-2 above, wherein the epitope may be made of a linear or non-linear sequence within VEGFR-2.

As previously stated, the antibody or fragment thereof in aspects is a sdAb. The sdAb may be of any origin, such as human or camelid origin or derived from a camelid Van, and thus may be based on camelid framework regions; alternatively, the CDR described above may be grafted onto V_{NAR}, V_{HH} or V_{L} framework regions.

The present embodiment further encompasses an antibody fragment that is “humanized” using any suitable method know in the art, for example, but not limited to CDR grafting and veneering. Humanization of an antibody or antibody fragment comprises replacing an amino acid in the sequence with its human counterpart, as found in the human consensus sequence, without loss of antigen-binding ability or specificity; this approach reduces immunogenicity of the antibody or fragment thereof when introduced into human subjects. In the process of CDR grafting, one or more than one of the heavy chain CDR defined herein may be fused or grafted to a human variable region (V_{H}, or V_{L}), or to other human antibody fragment framework regions (Fv, scFv, Fab). In such a case, the conformation of said one or more than one hypervariable loop is preserved, and the affinity and specificity of the sdAb for its target is also preserved.

CDR grafting is known in the art and is described in at least the following: U.S. Pat. No. 6,180,370, U.S. Pat. No. 5,693,761, U.S. Pat. No. 6,054,297, U.S. Pat. No. 5,859,205, and European Patent No. 626390. Veneering, also referred to in the art as “variable region resurfacing”, involves humanizing solvent-exposed positions of the antibody or fragment; thus, buried non-humanized residues, which may be important for CDR conformation, are preserved while the potential for immunological reaction against solvent-exposed regions is minimized. Veneering is known in the art and is described in at least the following: U.S. Pat. No. 5,869,619, U.S. Pat. No. 5,766,886, U.S. Pat. No. 5,821,123, and European Patent No. 519596. Persons of skill in the art would be amply familiar with methods of preparing such humanized antibody fragments.

In a specific, non-limiting example, the antibody or fragment thereof may comprise any one of the following sequences (note that sequences are also defined by their internal designations, e.g., AB1, V21, etc. in addition to their SEQ ID NO. These designations are used interchangeably herein, however, the SEQ ID NO should be considered the overriding definition if there is any question as to which sequence is being identified).

SEQ ID NO: 2 - AB1; V21; CDRs are underlined
MQVQLVESGG GLVQAGGSLR LSCAASGRAF SSYAMGWFRQ APGKERELVA AISWSDDSTY
YANSVKGRFT ISRDNAKSAV YLQMNSLKPE DTAVYYCAAH KSLQRPDEYT YWGQGTQVTV
SS
SEQ ID NO: 3 - V21H1; residues in bold are putative locations for
attachment to urease
MQVQLVESGG GLVQAGGSLR LSCAASGRAF SSYAMGWFRQ APGKERELVA AISWSDDSTY
YANSVKGRFT ISRDNAKSAV YLQMNSLKPE DTAVYYCAAH KSLQRPDEYT YWGQGTQVTV
SSGSEEEDDD GKK
SEQ ID NO: 4 - AB1 with linker; V21H2
MQVQLVESGG GLVQAGGSLR LSCAASGRAF SSYAMGWFRQ APGKERELVA AISWSDDSTY
YANSVKGRFT ISRDNAKSAV YLQMNSLKPE DTAVYYCAAH KSLQRPDEYT YWGQGTQVTV
SSGSEQKGGG EEDDG
SEQ ID NO: 5 - AB1m-2 with linker; V21H3
MKAIFVLKGS LDRDPEFDDE GGGQVQLVES GGGLVQAGGS LRLSCAASGR AFSSYAMGWF
RQAPGKEREL VAAISWSDDS TYYANSVKGR FTISRDNAKS AVYLQMNSLK PEDTAVYYCA
AHKSLQRPDE YTYWGQGTQV TVSSGSEQ
SEQ ID NO: 6 - AB1C: V21H4
MQVQLVESGG GLVQAGGSLR LSCAASGRAF SSYAMGWFRQ APGKERELVA AISWSDDSTY
YANSVKGRFT ISRDNAKSAV YLQMNSLKPE DTAVYYCAAH KSLQRPDEYT YWGQGTQVTV
SSGSEQKGGG EEDDGC
SEQ ID NO: 7 - AB1 with linker 2; VR2-21
MQVQLVESGG GLVQAGGSLR LSCAASGRAF SSYAMGWFRQ APGKERELVA AISWSDDSTY
YANSVKGRFT ISRDNAKSAV YLQMNSLKPE DTAVYYCAAH KSLQRPDEYT YWGQGTQVTV
SSGSEQKLIS EEDLNHHHHH H
SEQ ID NO: 8 - AB1m
MKKQVQLVES GGGLVQAGGS LRLSCAASGR AFSSYAMGWF RQAPGKEREL VAAISWSDDS
TYYANSVKGR FTISRDNAKS AVYLQMNSLK PEDTAVYYCA AHKSLQRPDE YTYWGQGTQV
TVSS
SEQ ID NO: 9 - AB1m with linker; V21N2K
MKKQVQLVES GGGLVQAGGS LRLSCAASGR AFSSYAMGWF RQAPGKEREL VAAISWSDDS
TYYANSVKGR FTISRDNAKS AVYLQMNSLK PEDTAVYYCA AHKSLQRPDE YTYWGQGTQV
TVSSGSEEED DDG
SEQ ID NO: 10 - AB1m-2
MKAIFVLKGS LDRDPEFDDE GGGQVQLVES GGGLVQAGGS LRLSCAASGR AFSSYAMGWF
RQAPGKEREL VAAISWSDDS TYYANSVKGR FTISRDNAKS AVYLQMNSLK PEDTAVYYCA
AHKSLQRPDE YTYWGQGTQV TVSS
SEQ ID NO: 11 - AB2; V18
MQVQLVESGG GLIKPGGSLR LSCAASGFRF SAESMTWVRQ APGKGLEWVS AISSSGGSTY
YADSVKGRFT ISRDNSKNTV YLQMNSLRAE DTAVYYCVRS PKGCTHASCS WNSGSWGQGT
LVTVSS
SEQ ID NO: 12 - AB2 with linker
MQVQLVESGG GLIKPGGSLR LSCAASGFRF SAESMTWVRQ APGKGLEWVS AISSSGGSTY
YADSVKGRFT ISRDNSKNTV YLQMNSLRAE DTAVYYCVRS PKGCTHASCS WNSGSWGQGT
LVTVSSGSEE DDDEEK
SEQ ID NO: 13 - AB2 with linker 2; VR2-801-18
MQVQLVESGG GLIKPGGSLR LSCAASGFRF SAESMTWVRQ APGKGLEWVS AISSSGGSTY
YADSVKGRFT ISRDNSKNTV YLQMNSLRAE DTAVYYCVRS PKGCTHASCS WNSGSWGQGT
LVTVSSGSEQ KLISEEDLNH HHHH
SEQ ID NO: 14 - V18H3
MQVQLVESGG GLIKPGGSLR LSCAASGFRF SAESMTWVRQ APGKGLEWVS AISSSGGSTY
YADSVKGRFT ISRDNSKNTV YLQMNSLRAE DTAVYYCVRS PKGCTHASCS WNSCSWGQGT
LVTVSSGSEQ KLISEEDLNG GGEDDEEGC
SEQ ID NO: 15 - AB2m
QVQLVESGGG LIKPGGSLRL SCAASGFRFS AESMTWVRQA PGKGLEWVSA ISSSGGSTYY
ADSVKGRFTI SRDNSKNTVY LQMNSLRAED TAVYYCVRSP KGCTHASCSW NSGSWGQGTL
VTVSS
SEQ ID NO: 16 - AB2m with linker
QVQLVESGGG LIKPGGSLRL SCAASGFRFS AESMTWVRQA PGKGLEWVSA ISSSGGSTYY
ADSVKGRFTI SRDNSKNTVY LQMNSLRAED TAVYYCVRSP KGCTHASCSW NSGSWGQGTL
VTVSSGSEQK LISEEDLNHH HHH
SEQ ID NO: 17 - AB2m-2
MKAIFVLKGS LDRDPEFDDE EGGGQVQLVE SGGGLIKPGG SLRLSCAASG FRFSAESMTW
VRQAPGKGLE WVSAISSSGG STYYADSVKG RFTISRDNSK NTVYLQMNSL RAEDTAVYYC
VRSPKGCTHA SCSWNSGSWG QGTLVTVSS
SEQ ID NO: 18 - AB2m-2 with linker; V18H2
MKAIFVLKGS LDRDPEFDDE EGGGQVQLVE SGGGLIKPGG SLRLSCAASG FRFSAESMTW
VRQAPGKGLE WVSAISSSGG STYYADSVKG RFTISRDNSK NTVYLQMNSL RAEDTAVYYC
VRSPKGCTHA SCSWNSGSWG QGTLVTVSSG SDEE
SEQ ID NO: 19 - AB3; V45
MQVQLVESGG GLIKPGGSLR LSCAASGDML SYDVMSWVRQ APGKGLEWVS AISSSGGSTY
YADSVKGRFT ISRDNSKNTV YLQMNSLRAE DTAVYYCVAA PWRCTHDNCS KTRASWGQGT
MVTVSS
SEQ ID NO: 20 - AB3 with linker; V45H1
MQVQLVESGG GLIKPGGSLR LSCAASGDML SYDVMSWVRQ APGKGLEWVS AISSSGGSTY
YADSVKGRFT ISRDNSKNTV YLQMNSLRAE DTAVYYCVAA PWRCTHDNCS KTRASWGQGT
MVTVSSGSEQ KGGGEEDDEE
SEQ ID NO: 21 - AB3 with linker 2; VR2-801-45
MQVQLVESGG GLIKPGGSLR LSCAASGDML SYDVMSWVRQ APGKGLEWVS AISSSGGSTY
YADSVKGRFT ISRDNSKNTV YLQMNSLRAE DTAVYYCVAA PWRCTHDNCS KTRASWGQGT
MVTVSSGSEQ KLISEEDLNH HHHH
SEQ ID NO: 22 - AB3m
MKKQVQLVES GGGLIKPGGS LRLSCAASGD MLSYDVMSWV RQAPGKGLEW VSAISSSGGS
TYYADSVKGR FTISRDNSKN TVYLQMNSLR AEDTAVYYCV AAPWRCTHDN CSKTRASWGQ
GTMVTVSS
SEQ ID NO: 23 - AB3m with linker; V45N2K
MKKQVQLVES GGGLIKPGGS LRLSCAASGD MLSYDVMSWV RQAPGKGLEW VSAISSSGGS
TYYADSVKGR FTISRDNSKN TVYLQMNSLR AEDTAVYYCV AAPWRCTHDN CSKTRASWCQ
GTMVTVSSGS EEEDDDG
SEQ ID NO: 24 - V45H2
MQVQLVESGG GLIKPGGSLR LSCAASGDML SYDVMSWVRQ APGKGLEWVS AISSSGGSTY
YADSVKGRFT ISRDNSKNTV YLQMNSLRAE DTAVYYCVAA PWRCTHDNCS KTRASWGQGT
MVTVSSGSEQ KLISEEDLNG GGEDEGC
SEQ ID NO: 25 - AB4; V38
MQVKLEESGG GLVQAGGSLR LSCAASGGTA SSYAMGWFRQ APGKEREFVA AISRSGGNTD
YVDSAKGRFT ISRDDAKNTV SLQMNSLRLE DTAVYYCAAR YAGTWPNDAG TVYWLPPNYN
YWGQGTQVTV SS
SEQ ID NO: 26 - AB4 with linker
MQVKLEESGG GLVQAGGSLR LSCAASGGTA SSYAMGWFRQ APGKEREFVA AISRSGGNTD
YVDSAKGRFT ISRDDAKNTV SLQMNSLRLE DTAVYYCAAR YAGTWPNDAG TVYWLPPNYN
YWGQGTQVTV SSGSEQ
SEQ ID NO: 27 - AB4 with linker 2; VR2-38
MQVKLEESGG GLVQAGGSLR LSCAASGGTA SSYAMGWFRQ APGKEREFVA AISRSGGNTD
YVDSAKGRFT ISRDDAKNTV SLQMNSLRLE DTAVYYCAAR YAGTWPNDAG TVYWLPPNYN
YWGQGTQVTV SSGSEQKLIS EEDLNHHHHH H
SEQ ID NO: 28 - AB4m
QVKLEESGGG LVQAGGSLRL SCAASGGTAS SYAMGWFRQA PGKEREFVAA ISRSGGNTDY
VDSAKGRFTI SRDDAKNTVS LQMNSLRLED TAVYYCAARY AGTWPNDAGT VYWLPPNYNY
WGQGTQVTVS S
SEQ ID NO: 29 - AB4m with linker
QVKLEESGGG LVQAGGSLRL SCAASGGTAS SYAMGWFRQA PGKEREFVAA ISRSGGNTDY
VDSAKGRFTI SRDDAKNTVS LQMNSLRLED TAVYYCAARY AGTWPNDAGT VYWLPPNYNY
WGQGTQVTVS SGSEQKLISE EDLNHHHHHH
SEQ ID NO: 30 - AB4c; V38H3
MQVKLEESGG GLVQAGGSLR LSCAASGGTA SSYAMGWFRQ APGKEREFVA AISRSGGNTD
YVDSAKGRFT ISRDDAKNTV SLQMNSLRLE DTAVYYCAAR YAGTWPNDAG TVYWLPPNYN
YWGQGTQVTV SSGSEQKGGG DEDGC

or a sequence at least 85%, at least 86%, at least 87%, at least 88%, at least 89%, at least 90%, at least 91%, at least 92%, at least 93%, at least 94% or at least 95% identical thereto, or a sequence substantially identical thereto.

These sequences may be coded by any nucleic acid sequence that would result in the recited amino acid sequence, as will be understood due to the degeneracy of the genetic code. Examples of nucleic acid sequences that may code the above-noted amino acid sequences include but are not limited to:

SEQ ID NO: 31 - AB1; V21
atgcaggtgc agctggtgga atccggcggc ggcctggtgc aggcgggcgg ctccctgcgt
ctgtcctgcg cggcgtccgg ccgtgcgttt tcctcctatg cgatgggctg gtttcgtcag
gcgccgggca aagaacgtga actggtggcg gcgatttcct ggtccgatga ttccacctat
tatgcgaatt ccgtgaaagg ccgttttacc atttcccgtg ataatgcgaa atccgcggtg
tatctacaga tgaattccct gaaaccggaa gataccgcgg tgtattattg cgcggcgcat
aaatccctac agcgtccgga tgaatatacc tattggggcc agggcaccca ggtgaccgtg
tcctcc
SEQ ID NO: 32 - AB1
atgcaggtgc agcttgtgga gtccggcgga ggtcttgtcc aggcaggagg gtctttgcgc
ctgagctgcg cggcgagtgg gcgcgcgttc agcagttacg cgatgggttg gttccgccag
gcccctggga aagagcgtga acttgtggct gccatttctt ggtctgatga ttccacctat
tatgctaatt cagttaaggg ccgtttcacg attagccgcg ataatgctaa atccgccgtc
tatcttcaga tgaacagcct taagcctgaa gatacggcag tatattattg tgccgctcat
aagagtctgc aacgcccgga cgaatataca tactggggac agggcacgca agttaccgtt
tccagc
SEQ ID NO: 33 - AB1 with linker; V21H2
atgcaggtgc agctggtgga atccggcggc ggcctggtgc aggcgggcgg ctccctgcgt
ctgtcctgcg cggcgtccgg ccgtgcgttt tcctcctatg cgatgggctg gtttcgtcag
gcgccgggca aagaacgtga actggtggcg gcgatttcct ggtccgatga ttccacctat
tatgcgaatt ccgtgaaagg ccgttttacc atttcccgtg ataatgcgaa atccgcggtg
tatctacaga tgaattccct gaaaccggaa gataccgcgg tgtattattg cgcggcgcat
aaatccctac agcgtccgga tgaatatacc tattggggcc agggcaccca ggtgaccgtg
tcctccggct ccgaacagaa aggcggcggc gaagaagatg atggc
SEQ ID NO: 34 - AB1 with linker 2; VR2-21
atgcaggtgc aactggttga atcaggtgga ggactggtgc aggccggggg atctttacgc
ttatcatgtg cagcttcggg gcgtgccttc tcctcttatg cgatgggatg gttccgccaa
gcccccggca aggagcgtga gctggtagca gccatttcct ggtcagacga cagtacctac
tacgcaaact cagtcaaagg gcgcttcact atctctcgcg acaatgccaa atccgctgtg
tacttgcaaa tgaactcatt gaagccagag gatacggctg tctattactg tgcagcccac
aagagtttac agcgtccaga tgaatacacc tattggggac aaggtacaca agttaccgtt
agttcgggta gcgaacaaaa gttgatctct gaggaggact taaatcatca tcatcatcac
cat
SEQ ID NO: 35 - AB1c; V21H4
atgcaggtgc agcttgtgga gtccggcgga ggtcttgtcc aggcaggagg gtctttgcgc
ctgagctgcg cggcgagtgg gcgcgcgttc agcagttacg cgatgggttg gttccgccag
gcccctggga aagagcgtga acttgtggct gccatttctt ggtctgatga ttccacctat
tatgctaatt cagttaaggg ccgtttcacg attagccgcg ataatgctaa atccgccgtc
tatcttcaga tgaacagcct taagcctgaa gatacggcag tatattattg tgccgctcat
aagagtctgc aacgcccgga cgaatataca tactggggac agggcacgca agttaccgtt
tccagcggtt ctgaacagaa aggaggcggt gaagaggatg atggctgc
SEQ ID NO: 36 - AB1m-2
atgaaagcga tcttcgttct gaaaggttct ctggaccgtg acccggaatt cgacgacgaa
ggtggtggtc aggttcagct ggttgaatct ggtggtggtc tggttcaggc gggtggttct
ctgcgtctgt cttgcgcggc gtctggtcgt gcgttctctt cttacgcgat gggttggttc
cgtcaggcgc cgggtaaaga acgtgaactg gttgcggcga tctcttggtc tgacgactct
acctactacg cgaactctgt taaaggtcgt ttcaccatct ctcgtgacaa cgcgaaatct
gcggtttacc tacagatgaa ctctctgaaa ccggaagaca ccgcggttta ctactgcgcg
gcgcacaaat ctctacagcg tccggacgaa tacacctact ggggtcaggg tacccaggtt
accgtttctt ct
SEQ ID NO: 37 - AB1m-2 with linker; V21H3
atgaaagcga tcttcgttct gaaaggttct ctggaccgtg acccggaatt cgacgacgaa
ggtggtggtc aggttcagct ggttgaatct ggtggtggtc tggttcaggc gggtggttct
ctgcgtctgt cttgcgcggc gtctggtcgt gcgttctctt cttacgcgat gggttggttc
cgtcaggcgc cgggtaaaga acgtgaactg gttgcggcga tctcttggtc tgacgactct
acctactacg cgaactctgt taaaggtcgt ttcaccatct ctcgtgacaa cgcgaaatct
gcggtttacc tacagatgaa ctctctgaaa ccggaagaca ccgcggttta ctactgcgcg
gcgcacaaat ctctacagcg tccggacgaa tacacctact ggggtcaggg tacccaggtt
accgtttctt ctggttctga acag
SEQ ID NO: 38 - AB2; V18
atgcaagttc agttagtaga aagtggtggt ggtttaatca aaccgggtgg ttcacttcgt
ttatcgtgcg cagcaagcgg gtttcgtttt tcagcagaat caatgacatg ggttcgtcaa
gcaccgggca aaggtttaga gtgggtttca gcaatttcat caagtggcgg ttcaacttat
tatgcagatt cggttaaagg tcgtttcaca atttctcgcg ataactcaaa aaatacggtt
tatttacaaa tgaattcctt acgtgcagaa gatacagcag tttattattg tgttcgttct
ccaaaaggtt gtactcacgc atcttgtagt tggaatagtg gtagttgggg tcaaggtaca
ttagttacag tctcaagc
SEQ ID NO: 39 - AB2
atgcaggtgc agttagttga gtcgggcggg ggtcttatta aaccaggtgg aagccttcgt
ctgtcttgtg cagcatcagg ctttcgtttt tccgcggaaa gcatgacctg ggtacgccaa
gcgcctggca aaggattgga gtgggtttcg gccatttctt cttcaggagg atcaacgtac
tatgcagact ccgtaaaagg acgcttcacg atttctcgcg ataactctaa gaacaccgtg
tacttacaaa tgaactcttt acgtgcagag gacacagcag tgtattattg tgttcgctca
cccaaaggct gcacccatgc gtcatgctct tggaactcag gttcgtgggg ccaggggacc
ttggtgacag tatcctcg
SEQ ID NO: 40 - AB2 with linker
atgcaagttc agttagtaga aagtggtggt ggtttaatca aaccgggtgg ttcacttcgt
ttatcgtgcg cagcaagcgg gtttcgtttt tcagcagaat caatgacatg ggttcgtcaa
gcaccgggca aaggtttaga gtgggtttca gcaatttcat caagtggcgg ttcaacttat
tatgcagatt cggttaaagg tcgtttcaca atttctcgcg ataactcaaa aaatacggtt
tatttacaaa tgaattcctt acgtgcagaa gatacagcag tttattattg tgttcgttct
ccaaaaggtt gtactcacgc atcttgtagt tggaatagtg gtagttgggg tcaaggtaca
ttagttacag tctcaagcgg ttcagaagaa gatgacgatg aagaaaaa
SEQ ID NO: 41 - AB2 with linker 2; VR2-801-18
atgcaggtgc agttagttga gtcgggcggg ggtcttatta aaccaggtgg aagccttcgt
ctgtcttgtg cagcatcagg ctttcgtttt tccgcggaaa gcatgacctg ggtacgccaa
gcgcctggca aaggattgga gtgggtttcg gccatttctt cttcaggagg atcaacgtac
tatgcagact ccgtaaaagg acgcttcacg atttctcgcg ataactctaa gaacaccgtg
tacttacaaa tgaactcttt acgtgcagag gacacagcag tgtattattg tgttcgctca
cccaaaggct gcacccatgc gtcatgctct tggaactcag gttcgtgggg ccaggggacc
ttggtgacag tatcctcggg ctccgaacag aagttaatta gtgaagaaga tttgaaccac
caccaccatc ac
SEQ ID NO: 42 - AB2m-2
atgaaagcga tcttcgttct gaaaggttct ctggaccgtg acccggaatt cgacgacgaa
gaaggtggtg gtcaggttca gctggttgaa tctggtggtg gtctgatcaa accgggtggt
tctctgcgtc tgtcttgcgc ggcgtctggt ttccgtttct ctgcggaatc tatgacctgg
gttcgtcagg cgccgggtaa aggtctggaa tgggtttctg cgatctcttc ttctggtggt
tctacctact acgcggactc tgttaaaggt cgtttcacca tctctcgtga caactctaaa
aacaccgttt acttacaaat gaactctctg cgtgcggaag acaccgcggt ttactactgc
gttcgttctc cgaaaggttg cacccacgcg tcttgctctt ggaactctgg ttcttggggt
cagggtaccc tggttaccgt ttcttct
SEQ ID NO: 43 - AB2m-2 with linker, V18H2
atgaaagcga tcttcgttct gaaaggttct ctggaccgtg acccggaatt cgacgacgaa
gaaggtggtg gtcaggttca gctggttgaa tctggtggtg gtctgatcaa accgggtggt
tctctgcgtc tgtcttgcgc ggcgtctggt ttccgtttct ctgcggaatc tatgacctgg
gttcgtcagg cgccgggtaa aggtctggaa tgggtttctg cgatctcttc ttctggtggt
tctacctact acgcggactc tgttaaaggt cgtttcacca tctctcgtga caactctaaa
aacaccgttt acttacaaat gaactctctg cgtgcggaag acaccgcggt ttactactgc
gttcgttctc cgaaaggttg cacccacgcg tcttgctctt ggaactctgg ttcttggggt
cagggtaccc tggttaccgt ttcttctggt tctgacgaag aa
SEQ ID NO: 44 - AB3, V45
atgcaggtgc agctggtgga aagcggcggc ggcctgatta aaccgggcgg cagcctgcgc
ctgagctgcg cggcgagcgg cgatatgctg agctatgatg tgatgagctg ggtgcgccag
gcgccgggca aaggcctgga atgggtgagc gcgattagca gcagcggcgg cagcacctat
tatgcggata gcgtgaaagg ccgctttacc attagccgcg ataacagcaa aaacaccgtg
tatcttcaga tgaacagcct gcgcgcggaa gataccgcgg tgtattattg cgtggcggcg
ccgtggcgct gcacccatga taactgctct aaaacccgcg cgagctgggg ccagggcacc
atggtgaccg tg
SEQ ID NO: 45 - AB3
atgcaagtac agttagtgga gagtggagga gggctgatta agccaggcgg ctctttgcgt
ctgagttgtg cggcatcagg cgatatgtta agctacgatg tgatgagttg ggtgcgtcaa
gcgccaggaa aaggacttga atgggtcagc gcaatttcgt cgtccggtgg gtctacttac
tacgctgatt cggttaaggg ccgcttcacc atctcccgcg acaattcaaa gaatacggta
tatctgcaaa tgaatagttt gcgtgcggag gacacagcag tctactattg cgttgcagct
ccctggcgct gtactcacga taactgttca aaaacccgcg catcatgggg tcaaggtaca
atggtgacag tgtcatct
SEQ ID NO: 46 - AB3 with linker; V45H1
atgcaggtgc agctggtgga aagcggcggc ggcctgatta aaccgggcgg cagcctgcgc
ctgagctgcg cggcgagcgg cgatatgctg agctatgatg tgatgagctg ggtgcgccag
gcgccgggca aaggcctgga atgggtgagc gcgattagca gcagcggcgg cagcacctat
tatgcggata gcgtgaaagg ccgctttacc attagccgcg ataacagcaa aaacaccgtg
tatcttcaga tgaacagcct gcgcgcggaa gataccgcgg tgtattattg cgtggcggcg
ccgtggcgct gcacccatga taactgctct aaaacccgcg cgagctgggg ccagggcacc
atggtgaccg tgagcagcgg cagcgaacag aaaggcggcg gcgaagaaga tgatgaagaa
SEQ ID NO: 47 - AB3 with linker 2; VR2-801-45
atgcaagtac agttagtgga gagtggagga gggctgatta agccaggcgg ctctttgcgt
ctgagttgtg cggcatcagg cgatatgtta agctacgatg tgatgagttg ggtgcgtcaa
gcgccaggaa aaggacttga atgggtcagc gcaatttcgt cgtccggtgg gtctacttac
tacgctgatt cggttaaggg ccgcttcacc atctcccgcg acaattcaaa gaatacggta
tatctgcaaa tgaatagttt gcgtgcggag gacacagcag tctactattg cgttgcagct
ccctggcgct gtactcacga taactgttca aaaacccgcg catcatgggg tcaaggtaca
atggtgacag tgtcatctgg tagtgaacag aagttaatta gtgaagagga ccttaatcat
catcatcatc ac
SEQ ID NO: 48 - V45H2
atgcaggttc agctggttga atctggtggt ggtctgatca aaccgggtgg ttctctgcgt
ctgtcttgcg cggcgtctgg tgacatgctg tcttacgacg ttatgtcttg ggttcgtcag
gcgccgggta aaggtctgga atgggtttct gcgatctctt cttctggtgg ttctacctac
tacgcggact ctgttaaagg tcgtttcacc atctctcgtg acaactctaa aaacaccgtt
tacctgcaaa tgaactctct gcgtgcggaa gacaccgcgg tttactactg cgttgcggcg
ccgtggcgtt gcacccacga caactgctct aaaacccgtg cgtcttgggg tcagggtacc
atggttaccg tttcttctgg ttctgaacag aaactgatct ctgaagaaga cctgaacggt
ggtggtgaag acgaaggttg c
SEQ ID NO: 49 - AB4; V38
atgcaagtaa aactcgaaga atcaggtgga ggattggttc aagctggtgg gtcattacgt
ttgtcctgtg cagcaagtgg cggtactgcg tcaagttatg caatgggttg gtttcgtcaa
gctcccggta aagaacgtga atttgttgcc gcaattagtc ggtccggagg aaatacagat
tatgtagact cagcaaaagg tcgttttact atctcacgcg atgatgcaaa aaatacggtt
tccttacaaa tgaactctct gcgcctcgaa gataccgcgg tatattattg cgctgcccgc
tacgccggta cctggccgaa tgatgctggc actgtatatt ggctgccacc gaattacaac
tattggggtc aaggaactca agtcacggta agcagc
SEQ ID NO: 50 - AB4
atgcaggtta aattagagga atcaggtgga ggtttggttc aagcaggtgg tagcttgcgc
ctgagttgtg ccgctagcgg gggcacagcc agttcatacg cgatggggtg gtttcgccag
gcccctggaa aggagcgtga attcgttgct gcgattagtc gtagcggcgg taacacggat
tacgtggaca gcgcgaaggg acgctttaca atttctcgtg atgacgcaaa gaacacggtg
tccctgcaaa tgaactcact tcgcctggaa gacaccgcgg tgtattattg tgcagcccgc
tacgcgggaa cttggccgaa cgatgctggt accgtgtact ggttaccccc taattacaat
tactggggcc aaggtaccca agtcaccgtc tcctcg
SEQ ID NO: 51 - AB4 with linker
atgcaagtaa aactcgaaga atcaggtgga ggattggttc aagctggtgg gtcattacgt
ttgtcctgtg cagcaagtgg cggtactgcg tcaagttatg caatgggttg gtttcgtcaa
gctcccggta aagaacgtga atttgttgcc gcaattagtc ggtccggagg aaatacagat
tatgtagact cagcaaaagg tcgttttact atctcacgcg atgatgcaaa aaatacggtt
tccttacaaa tgaactctct gcgcctcgaa gataccgcgg tatattattg cgctgcccgc
tacgccggta cctggccgaa tgatgctggc actgtatatt ggctgccacc gaattacaac
tattggggtc aaggaactca agtcacggta agcagcggtt ccgaacaaaa gggtggtgga
gaagaagatg atggcaaa
SEQ ID NO: 52 - AB4 with linker 2; VR2-38
atgcaggtta aattagagga atcaggtgga ggtttggttc aagcaggtgg tagcttgcgc
ctgagttgtg ccgctagcgg gggcacagcc agttcatacg cgatggggtg gtttcgccag
gcccctggaa aggagcgtga attcgttgct gcgattagtc gtagcggcgg taacacggat
tacgtggaca gcgcgaaggg acgctttaca atttctcgtg atgacgcaaa gaacacggtg
tccctgcaaa tgaactcact tcgcctggaa gacaccgcgg tgtattattg tgcagcccgc
tacgcgggaa cttggccgaa cgatgctggt accgtgtact ggttaccccc taattacaat
tactggggcc aaggtaccca agtcaccgtc tcctcgggaa gcgaacaaaa gctgattagc
gaagaggatc ttaaccatca tcatcaccat cac
SEQ ID NO: 53 - AB4c; V38H3
atgcaggtta aactggaaga atctggtggt ggtctggttc aggcgggtgg ttctctgcgt
ctgtcttgcg cggcgtctgg tggtaccgcg tcttcttacg cgatgggttg gttccgtcag
gcgccgggta aagaacgtga attcgttgcg gcgatctctc gttctggtgg taacaccgac
tacgttgact ctgcgaaagg tcgtttcacc atctctcgtg acgacgcgaa aaacaccgtt
tctctgcaaa tgaactctct gcgtctggaa gacaccgcgg tttactactg cgcggcgcgt
tacgcgggta cctggccgaa cgacgcgggt accgtttact ggctgccgcc gaactacaac
tactggggtc agggtaccca ggttaccgtt tcttctggtt ctgaacagaa aggtggtggt
gacgaagacg gttgc

or a sequence at least 85%, at least 86%, at least 87%, at least 88%, at least 89%, at least 90%, at least 91%, at least 92%, at least 93%, at least 94% or at least 95% identical thereto, or a sequence substantially identical thereto.

Linker sequences suitable for the single domain antibodies of the invention may be selected from the group consisting of GSEQ (SEQ ID NO:54), GSDEE (SEQ ID NO:55), GSEEEDDDG (SEQ ID NO:56), GSEEEDDDGKK (SEQ ID NO:57), GSEQKGGGEEDDG (SEQ ID NO:58), GSEQKLISEEDLNHHHHH (SEQ ID NO:59), GSEQKLISEEDLNHHHHHH (SEQ ID NO:60), GSEEDDDEEK (SEQ ID NO:61), GSEQKGGGEEDDEE (SEQ ID NO:62), GSEQKLISEEDLNGGGEDDEEG (SEQ ID NO:63), GSEQKLISEEDLNGGGEDEG (SEQ ID NO:64), and GSEQKGGGDEDG (SEQ ID NO:65). In aspects, a linker sequence may further comprise a C-terminal cysteine, for example GSEQKGGGEEDDGC (SEQ ID NO:66), GSEQKLISEEDLNGGGEDDEEGC (SEQ ID NO:67), GSEQKLISEEDLNGGGEDEGC (SEQ ID NO:68), and GSEQKGGGDEDGC (SEQ ID NO:69). Sequences similar to these linker sequences may be used herein. For example, KK is a suitable linker sequence and those comprising any one of the sequences of SEQ ID NO:54-69.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The single domain antibody or fragment thereof of the present invention may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection tag, exemplary tag cassettes include Strep tag, or any variant thereof; see, e.g., U.S. Pat. No. 7,981,632, His tag. Flag tag having the sequence motif DYKDDDDK (SEQ ID NO:70), Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprises an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

The antibody or fragment thereof of the present invention may also be in a multivalent display. Multimerization may be achieved by any suitable method of known in the art. For example, and without wishing to be limiting in any manner, multimerization may be achieved using self-assembly molecules as described in Zhang et al (2004a; 2004b) and WO2003/046560.

The described method produces pentabodies by expressing a fusion protein comprising the antibody or fragment thereof of the present invention and the pentamerization domain of the B-subunit of an AB₅ toxin family (Merritt & Hol, 1995); the pentamerization domain assembles into a pentamer, through which a multivalent display of the antibody or fragment thereof is formed. Additionally, the pentamerization domain may be linked to the antibody or antibody fragment using a linker; such a linker should be of sufficient length and appropriate composition to provide flexible attachment of the two molecules, but should not hamper the antigen-binding properties of the antibody.

Other forms of multivalent display are also encompassed by the present invention. For example, and without wishing to be limiting, the antibody or fragment thereof may be presented as a dimer, a trimer, or any other suitable oligomer. This may be achieved by methods known in the art, for example direct linking connection (Nielson et al, 2000), c-jun/Fos interaction (de Kruif & Logtenberg, 1996), “Knob into holes” interaction (Ridgway et al, 1996).

Another method known in the art for multimerization is to dimerize the antibody or fragment thereof using an Fc domain. When applied in vivo, sdAbs are cleared quickly from the circulation (Bell et al., 2010). To solve this problem and to give sdAbs the ability to induce immune response after antigen binding, sdAbs may be fused to human Fc to generate chimeric heavy chain antibodies (Bell et al. Cancer Letters, 2010). In this approach, the Fc gene in inserted into a vector along with the sdAb gene to generate a sdAb-Fc fusion protein (Bell et al, 2010; Iqbal et al, 2010); the fusion protein is recombinantly expressed then purified. Such antibodies are easy to engineer and to produce (Zhang et al, 2009b), can greatly extend the serum half1 life of sdAbs, and may be excellent tumor imaging reagents (Bell et al., Cancer Letters, 2010).

The Fc domain in the multimeric complex as just described may be any suitable Fc fragment known in the art. The Fc fragment may be from any suitable source; for example, the Fc may be of mouse or human origin. In a specific, non-limiting example, the Fc may be the mouse Fc2b fragment or human Fc1 fragment (Bell et al, 2010; Iqbal et al, 2010).

The present invention further encompasses the isolated or purified antibody or fragments thereof immobilized onto a surface using various methodologies; for example, and without wishing to be limiting, the antibody or fragment may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

The present invention further provides a single domain antibody or fragment thereof linked to a cargo molecule; the antibody or fragment thereof may deliver the cargo molecule to a desired site. The single domain antibody or fragment thereof may be linked to the cargo molecule using any method known in the art (recombinant technology, chemical conjugation, chelation, etc.). The cargo molecule may be any type of molecule that may diagnose or reduce/inhibit the growth of tumours. Thus, the cargo molecule may be linked to a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer, an enzyme; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the antibody or fragment thereof may be linked to a fluorescent agent such as FITC or may genetically be fused to the Enhanced Green Fluorescent Protein (EGFP).

The antibodies of the present invention linked to a diagnostic agent, also referred to herein as a molecular imaging agent, may be used to perform diagnostic imaging. The imaging technique may include whole body imaging for diagnostic purposes or local imaging at specific sites, such as but not limited to sites of tumor growth, in a quantitative manner to assess the progression of disease or host response to a treatment regimen. The imaging may be accomplished by in vitro or in vivo by any suitable method known in the art. For example, and without wishing to be limiting, the diagnostic imaging technique may include immunohistochemistry, immunofluorescence staining, or a non-invasive (molecular) diagnostic imaging technology including, but not limited to: Optical imaging; Positron emission tomography (PET); Single photon emission computed tomography (SPECT); Magnetic resonance imaging (MRI), iron oxide nanoparticles and carbon-coated iron-cobalt nanoparticles.

The present invention also provides an in vivo method of detecting tumors, comprising: a) administering to a subject the single domain antibody or fragment thereof described herein linked to a diagnostic agent; and b) detecting the binding of the antibody or fragment thereof.

In the in vivo method as described above, the diagnostic agent may be radioisotope, a paramagnetic label, a fluorophore, a Near Infra-Red (NIR) fluorochrome or dye, an affinity label, or a detectable protein-based molecule via genetic fusion to the antibody, or other suitable agent as described above. In the method as just described, the step of detecting (step b)) may be accomplished by any appropriate imaging method including, but not limited to non-invasive optical imaging, ultrasound. MRI. PET, or SPECT, or other suitable method.

The present invention further provides an in vitro method of tumor diagnostics, comprising: a) contacting a tumor sample with the isolated or purified single domain antibody or fragment thereof linked to a diagnostic agent, as described herein; and b) detecting the binding of the isolated or purified antibody or fragment thereof.

The present invention also provides a method of blocking VEGFR-2 and decrease its activation leading to reducing the ability of tumor cells to promote angiogenesis. The method comprises administering any one or more of the antibodies as disclosed herein, or fragments thereof or a combination thereof to a subject in need thereof.

The sdAbs against VEGFR-2 are candidates for the development of antibody-based drugs against cancers and vascularization of tumors. The sdAb of the present invention or fragments thereof can block VEGFR-2 and decrease its activation. Such treatment may reduce the ability of the tumor cells to promote cell angiogenesis. An advantage of these antibodies over drugs used for chemotherapy is that they are more specific for tumors that over-express VEGFR-2.

Additionally, in aspects, single-domain antibodies such as those of SEQ ID NO:2-30, or fragments thereof are known to possess stability; they show ease in antibody engineering; and have superior tissue penetration ability due to their small size. The Fc-fusion versions comprising linker sequences such as SEQ ID NO:54-69 or fragments thereof are also advantageous for increasing half-life in circulation.

Single domain anti-VEGFR-2 antibodies of the present invention specifically bind to VEGFR-2. Antibody specificity, which refers to selective recognition of an antibody for a particular epitope of an antigen, of antibodies for VEGFR-2 can be determined based on affinity and/or avidity. Affinity, represented by the equilibrium constant for the dissociation of an antigen with an antibody (K_{d}), measures the binding strength between an antigenic determinant (epitope) and an antibody binding site. Avidity is the measure of the strength of binding between an antibody with its antigen. Antibodies typically bind with a K_{d} of 10⁻⁵ to 10⁻¹ l liters/mole. Any K_{d} greater than 10⁻⁴ liters/mole is generally considered to indicate non-specific binding. The lesser the value of the K_{d}, the stronger the binding strength between an antigenic determinant and the antibody binding site. In aspects, the antibodies described herein have a K_{d} of less than 10⁻⁴ L/mol, 10⁻⁵ L/mol, 10⁻⁶ L/mol, 10⁻⁷ L/mol, 10⁻⁸ L/mol, or 10⁻⁹ L/mol. In most preferred aspects a K_{d} of less than 10⁻⁴ L/mol.

Anti-VEGFR-2 antibodies of the present invention specifically bind to the extracellular region of VEGFR-2 and may neutralize activation of VEGFR-2 by preventing binding of a ligand of VEGFR-2 to the receptor. In such embodiments, the antibody binds VEGFR-2 at least as strongly as the natural ligands of VEGFR-2 (for example, VEGF(A)(E)(C) and (D)).

Neutralizing activation of VEGFR-2 includes diminishing, inhibiting, inactivating, and/or disrupting one or more of the activities associated with signal transduction. Such activities include receptor dimerization, autophosphorylation of VEGFR-2, activation of VEGFR-2's internal cytoplasmic tyrosine kinase domain, and initiation of multiple signal transduction and transactivation pathways involved in regulation of DNA synthesis (gene activation) and cell cycle progression or division. One measure of VEGFR-2 neutralization is inhibition of the tyrosine kinase activity of VEGFR-2. Tyrosine kinase inhibition can be determined using well-known methods such as phosphorylation assays which measuring the autophosphorylation level of recombinant kinase receptor, and/or phosphorylation of natural or synthetic substrates. Phosphorylation can be detected, for example, using an antibody specific for phosphotyrosine in an ELISA assay or on a western blot. Some assays for tyrosine kinase activity are described in Panek et al., J. Pharmacol. Exp. Them., 283: 1433-44 (1997) and Batley et al, Life ScL. 62: 143-50 (1998), both of which are incorporated by reference.

In addition, methods for detection of protein expression can be utilized to determine whether an antibody neutralizes activation of VEGFR-2, wherein the proteins being measured are regulated by VEGFR-2 tyrosine kinase activity. These methods include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, 78:1284-92. (1996); Shimizu et al., Japan J. Cancer Res., 85:567-71 (1994); Sauteret al., Am. J. Path., 148:1047-53 (1996); Collins, Glia, 15:289-96 (1995); Radinsky et al., Clin. Cancer Res., 1:19-31 (1995); Petrides et al., Cancer Res., 50:3934-39 (1990); Hoffmann et al., Anticancer Res., 17:4419-26 (1997); Wikstrand et al., Cancer Res., 55:3140-48 (1995), all of which are incorporated by reference.

In vivo assays can also be utilized to detect VEGFR-2 neutralization. For example, receptor tyrosine kinase inhibition can be observed by mitogenic assays using cell lines stimulated with receptor ligand in the presence and absence of inhibitor. For example, HUVEC cells (ATCC) stimulated with VEGF(A) or VEGF-B can be used to assay VEGFR-2 inhibition. Another method involves testing for inhibition of growth of VEGF-expressing tumor cells, using for example, human tumor cells injected into a mouse. See e.g., U.S. Pat. No. 6,365,157 (Rockwell et al.), which is incorporated by reference herein.

The present invention is not limited by any particular mechanism of VEGFR-2 neutralization. The single domain anti-VEGFR-2 antibodies of the present invention may, for example, bind externally to VEGFR-2, block and/or compete for binding of ligand to VEGFR-2 and inhibit subsequent signal transduction mediated via receptor-associated tyrosine kinase, and prevent phosphorylation of VEGFR-2 and other downstream proteins in the signal transduction cascade. The receptor-antibody complex may also be internalized and degraded, resulting in receptor cell surface down-regulation.

Polynucleotides encoding anti-VEGFR-2 antibodies of the present invention include polynucleotides with nucleic acid sequences that are substantially the same as the nucleic acid sequences of the polynucleotides of the present invention selected from any one of SEQ ID NO:31-53. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of DNAs that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, the present invention provides expression vectors containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colE1, pCR1, pBR322, pMB9, pUC, pKSM, and RP4. Prokaryotic vectors also include derivatives of phage DNA such as M13 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp. Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., PhoS, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

The present invention also provides recombinant host cells containing the expression vectors previously described. Single domain anti-VEGFR-2 antibodies of the present invention can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce sdAbs by culturing the cells under conditions permitting expression of the antibody and purifying the antibody from the host cell or medium surrounding the host cell. Targeting of the expressed antibody for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The anti-VEGFR-2 single domain antibodies of the present invention can be fused to additional amino acid residues. Such amino acid residues can be a peptide tag to facilitate isolation, for example. Other amino acid residues for homing of the antibodies to specific organs or tissues are also contemplated.

In another embodiment, the present invention provides methods of treating cancer by administering a therapeutically effective amount of a single domain anti-VEGFR-2 single domain antibody according to the present invention to a mammal in need thereof. Therapeutically effective means an amount effective to produce the desired therapeutic effect, such as reducing angiogenesis and/or decreasing or slowing down tumor growth.

In an aspect, the present invention provides a method of reducing tumor growth or inhibiting angiogenesis by administering a therapeutically effective amount of a single domain anti-VEGFR-2 antibody of the present invention to a mammal in need thereof.

With respect to reducing tumor growth, such tumors include primary tumors and metastatic tumors, as well as refractory tumors. Refractory tumors include tumors that fail to respond or are resistant to other forms of treatment such as treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued.

Single domain anti-VEGFR-2 antibodies of the present invention are useful for treating tumors that express VEGFR-2. Such tumors are characteristically sensitive to VEGF present in their environment, and may further produce and be stimulated by VEGF in an autocrine stimulatory loop. The method is therefore effective for treating a solid or non-solid tumor that is not vascularized, or is not yet substantially vascularized.

Examples of solid tumors which may be accordingly treated include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors.

With respect to inhibiting angiogenesis, the single domain anti-VEGFR-2 antibodies of the present invention are effective for treating subjects with vascularized tumors or neoplasms, or angiogenic diseases characterized by excessive angiogenesis. The antibodies described herein are also effective, in aspects, for preventing vascularization of primary or metastatic tumors. Such tumors and neoplasms include, for example, malignant tumors and neoplasms, such as blastomas, carcinomas or sarcomas, and highly vascular tumors and neoplasms. Cancers that may be treated by the methods of the present invention include, for example, cancers of the brain, genitourinary tract, lymphatic system, stomach, renal, colon, larynx and lung and bone. Non-limiting examples further include epidermoid tumors, squamous tumors, such as head and neck tumors, colorectal tumors, prostate tumors, breast tumors, lung tumors, including lung adenocarcinoma and small cell and non-small cell lung tumors, pancreatic tumors, thyroid tumors, ovarian tumors, and liver tumors.

Non-limiting examples of pathological angiogenic conditions characterized by excessive angiogenesis involving, for example inflammation and/or vascularization include atherosclerosis, rheumatoid arthritis (RA), neovascular glaucoma, proliferative retinopathy including proliferative diabetic retinopathy, macular degeneration, hemangiomas, angiofibromas, and psoriasis. Other non-limiting examples of non-neoplastic angiogenic disease are retinopathy of prematurity (retrolental fibroplastic), corneal graft rejection, insulin-dependent diabetes mellitus, multiple sclerosis, myasthenia gravis. Crohn's disease, autoimmune nephritis, primary biliary cirrhosis, psoriasis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osier-Weber syndrome, restinosis, and fungal, parasitic and viral infections, including cytomegaloviral infections.

The identification of medical conditions treatable by the single domain anti-VEGFR-2 antibodies of the present invention is well within the ability and knowledge of one skilled in the art. For example, human individuals who are either suffering from a clinically significant neoplastic or angiogenic disease or who are at risk of developing clinically significant symptoms are suitable for administration of the present VEGF receptor antibodies. A clinician skilled in the art can readily determine, for example, by the use of clinical tests, physical examination and medical/family history, if an individual is a candidate for such treatment.

Single domain anti-VEGFR-2 antibodies of the present invention can be administered for therapeutic treatments to a patient suffering from a tumor or angiogenesis associated pathologic condition in an amount sufficient to prevent, inhibit, or reduce the progression of the tumor or pathologic condition. Progression includes, e.g, the growth, invasiveness, metastases and/or recurrence of the tumor or pathologic condition. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. It should be noted, however, that the present invention is not limited to any particular dose.

In another embodiment, the present invention provides a method of treating a condition where decreased angiogenesis is desired by administering single domain anti-VEGFR-2 antibody of the present invention in combination with one or more other agents. For example, an embodiment of the present invention provides a method of treating such a condition by administering a single domain anti-VEGFR-2 antibody of the present invention with an antineoplastic or antiangiogenic agent. The single domain anti-VEGFR-2 antibody can be chemically or biosynthetically linked to one or more of the antineoplastic or antiangiogenic agents.

Any suitable antineoplastic agent can be used, such as a chemotherapeutic agent or radiation. Examples of chemotherapeutic agents include, but are not limited to, cisplatin, carboplatin, pemetrexed, doxorubicin, cyclophosphamide, paclitaxel, irinotecan (CPT-II), topotecan or a combination thereof. When the antineoplastic agent is radiation, the source of the radiation can be either external (external beam radiation therapy—EBRT) or internal (brachytherapy—BT) to the patient being treated.

Further, the present invention provides a method of treating a medical condition by administering a single domain anti-VEGFR-2 antibody of the present invention in combination with one or more suitable adjuvants, such as, for example, cytokines (IL-I0 and IL-13, for example) or other immune stimulators.

In a combination therapy, the single domain anti-VEGFR-2 antibodies of the invention can be administered before, during, or after commencing therapy with another agent, as well as any combination thereof, i.e., before and during, before and after, during and after, or before, during and after commencing the antineoplastic agent therapy. For example, a single domain anti-VEGFR-2 antibody of the present invention may be administered between 1 and 30 days, typically 3 and 20 days, more typically between 5 and 12 days before commencing radiation therapy. The present invention, however is not limited to any particular administration schedule. The dose of the other agent administered depends on numerous factors, including, for example, the type of agent, the type and severity of the medical condition being treated and the route of administration of the agent. The present invention, however, is not limited to any particular dose.

Any suitable method or route can be used to administer the single domain anti-VEGFR-2 antibodies of the present invention, and optionally, to co-administer antineoplastic agents and/or antagonists of other receptors. Routes of administration include, for example, oral, intravenous, intraperitoneal, subcutaneous, or intramuscular administration. It should be emphasized, however, that the present invention is not limited to any particular method or route of administration.

It is noted that a single domain anti-VEGFR-2 antibodies of the present invention can be administered as a conjugate, which binds specifically to the receptor and delivers a toxic, lethal payload following ligand-toxin internalization.

Compositions are provided that include one or more of the disclosed antibodies that bind (for example specifically bind) VEGFR-2 in a carrier. Compositions comprising fusion proteins, immunoconjugates or immunotoxins are also provided. The compositions can be prepared in unit dosage forms for administration to a subject. The amount and timing of administration are at the discretion of the treating clinician to achieve the desired outcome. The antibody or combination of antibodies can be formulated for systemic or local (such as intra-tumor) administration. In one example, the antibody is formulated for parenteral administration, such as intravenous administration.

The compositions for administration can include a solution of the antibody dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of antibody per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and in some cases administered at a dosage of from 0.5 to 15 mg/kg of body weight. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Administration of the antibodies, fusion proteins and immunoconjugates (or compositions thereof) disclosed herein can also be accompanied by administration of other anti-cancer agents or therapeutic treatments (such as surgical resection of a tumor). Any suitable anti-cancer agent can be administered in combination with the antibodies, compositions, fusion proteins and immunoconjugates disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

It is understood that the single domain anti-VEGFR-2 antibodies of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies of the invention are particularly useful for administration to humans, they may be administered to other mammals as well. The term “mammal” as used herein is intended to include, but is not limited to, humans, laboratory animals, domestic pets and farm animals.

The present invention also includes kits for inhibiting tumor growth and/or angiogenesis comprising a therapeutically effective amount of a single domain anti-VEGFR-2 antibody of the present invention. The kits can further contain any suitable antagonist of, for example, another growth factor receptor involved in tumorigenesis or angiogenesis. Alternatively, or in addition, the kits of the present invention can further comprise an antineoplastic agent. Examples of suitable antineoplastic agents in the context of the present invention have been described herein. The kits of the present invention can further comprise an adjuvant, examples of which have also been described above. Kits may include instructions.

In another embodiment, the present invention provides investigative or diagnostic methods using the single domain anti-VEGFR-2 antibodies of the present invention in vivo or in vitro. In such methods, anti-VEGFR-2 antibodies can be linked to target or reporter moieties.

The present invention will be further illustrated in the following examples. However, it is to be understood that these examples are for illustrative purposes only and should not be used to limit the scope of the present invention in any manner.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### Example 1: Generation of Anti-VEGFR-2 Antibodies

To generate camelid single domain antibodies targeting the extracellular domain of VEGFR-2, a llama was immunized with recombinant VEGFR-2/Fc. A phage display library was generated and screened to identify single domain antibodies with high binding affinity to VEGFR-2.

To generate human single domain antibodies targeting the extracellular domain of VEGFR-2, a human VH library was screened to identify single domain antibodies with high binding affinity to VEGFR-2.

A fusion partner sequence MKAIFVLKGSLDRDPEFDDE (SEQ ID NO:71) was added to the N-terminus of SEQ ID NO:2 and SEQ ID NO: 11 (AB1 and AB2) sequences to increase the yield of the antibody by accumulating the expressed proteins in inclusion bodies and effectively simplifying protein purification and refolding processes.

Four antibodies were made and further studied. The selected antibodies were expressed in the E. coli. BL21 (DE3) pT7 system. Two of these antibodies (AB2 (SEQ ID NO:13) and AB3 (SEQ ID NO:21)) are based on a human antibody scaffold and two SEQ ID NO:7 and 27 (AB1 and AB4) are of llama origin. These antibodies displayed binding kinetics that are of sufficient quality to be considered potential candidates for specific VEGFR-2 binding (Table 1).

TABLE 1
Characterization of antibodies.

| | | Kinetic Constants | | | Rmax |
|---|---|---|---|---|---|
| Antibody | Origin | ka (1/Ms) | kd (1/s) | K_{D} (M) | (RU) |
| AB2 SEQ | Human | 4.6 × 10⁴ | 0.02 | 5 × 10⁻⁷ | 1100 |
| ID NO: 13 | | | | | |
| AB3 SEQ | Human | 5.3 × 10⁴ | 0.045 | 9 × 10⁻⁷ | 1100 |
| ID NO: 21 | | | | | |
| AB1 SEQ | Llama | Approximately | <0.01 | Approximately | −700 |
| ID NO: 7 | | 6 × 10⁴ | | 8 × 10⁻⁸ | |
| AB4 SEQ | Llama | 2 × 10⁴ | 0.015 | 8 × 10⁻⁸ | 370 |
| ID NO: 27 | | | | | |

### Example 2: Human VEGFR-2/Fc Binders

The binding kinetics for the interactions of human SEQ ID NO:13 (AB2) and SEQ ID NO:21 (AB3) and llama SEQ ID NO:7 (AB1) and SEQ ID NO:27 (AB4) to immobilized human and mouse VEGFR-2/Fc were determined by SPR using a Biacore 3000 system, 12,000 RUs of human VEGFR2/Fc (R&D Systems), 14,000 RUs of mouse VEGFR-2/Fc (R& D Systems), or 7500 RUs of BSA (Sigma) as a reference protein were immobilized on research grade CM5-sensorchips (Biacore), respectively. Immobilizations were carried out at a protein concentration of 50 μg/ml in 10 mM Acetate pH 4.5 using an amine coupling kit supplied by the manufacturer. All antibody samples were passed though a Superdex 75 column (GE Healthcare) to separate monomer forms subject to Biacore analysis.

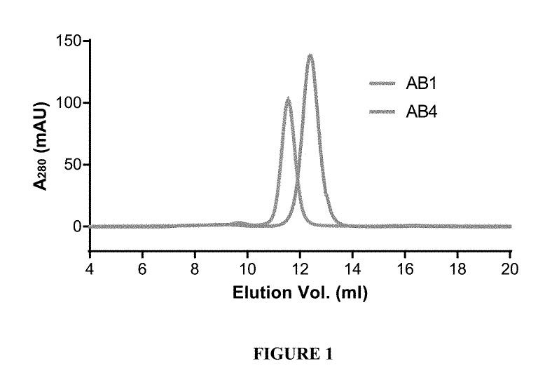

In all instances, analyses were carried out at 25° C. in 10 mM HEPES. pH 7.4 containing 150 mM NaCl, 3 mM EDTA and 0.005% surfactant P20 at a flow rate of 40 μl/min. The surfaces were regenerated with 3-8 sec contact time of 10 mM HCl. Data were analyzed with BIAevaluation 4.1 software. All four antibodies showed mainly monomer peaks. (FIG. 1, size exclusion column chromatograms). Conditions for size exclusion column chromatography: Machine: AKTÅ FPLC (GE healthcare); Superdex 75 HR 10/30 column (Amersham, Cat. No. 17-1047-01, Id No. 9937116); Running buffer: HBS-EP (10 mM HEPES, 150 mM NaCl, 3 mM EDTA, pH7.4, 0.005% P20); and 4×HBS-E was diluted 4 times and 10% P20 surfactant was added to make final 0.005%. Sample volume: 200 μl. Pump speed: 0.5 ml/min.

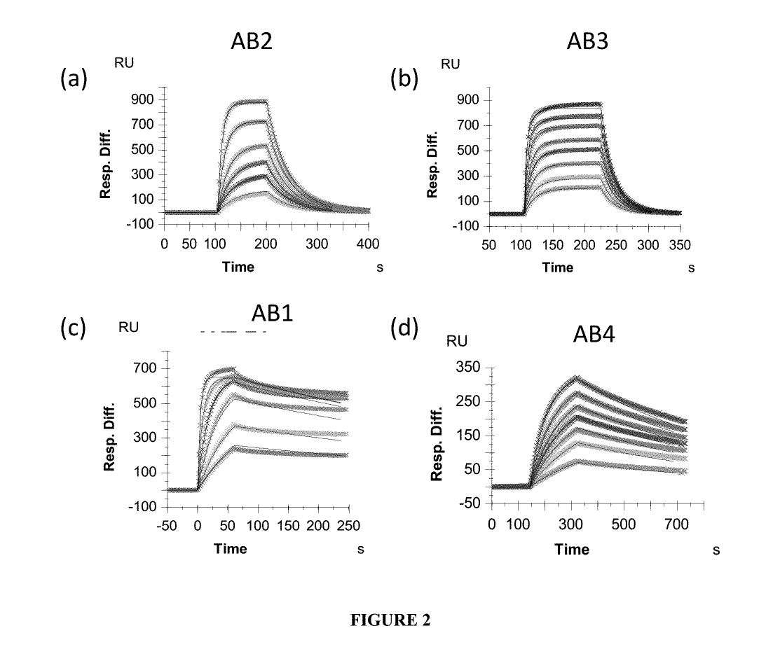

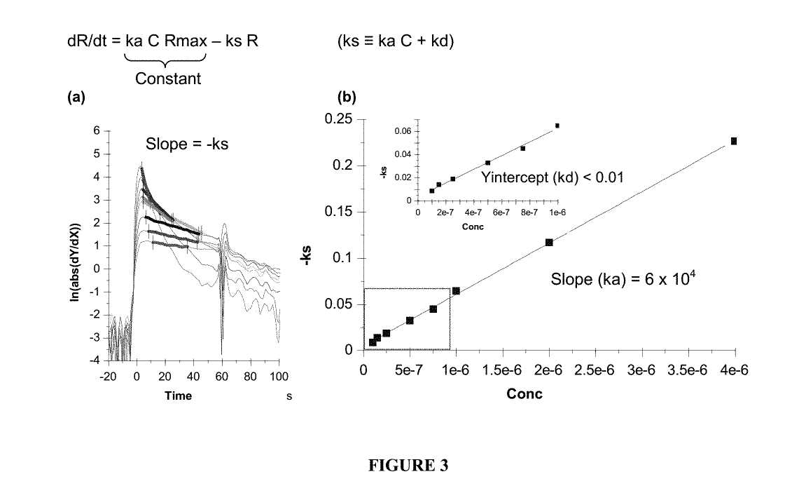

None of the antibodies showed binding to immobilized mouse VEGFR-2/Fc at the concentration of 150-200 nM, whereas all showed binding to immobilized human VEGFR-2/Fc (Table 1 and FIG. 2). These data indicate that the antibodies are species-specific. SEQ ID NO:7 (AB1) dissociates poorly on the SPR surface and complicated a direct fit of the sensorgram data (FIG. 2) to standard kinetic models. Therefore SEQ ID NO:7 (AB1) kinetic constants were estimated from transformed data shown in FIG. 3.

### Example 3: Human & Llama Antibodies Binding to Human VEGFR-2/Fc

Sensorgram overlays showing the binding of (a) SEQ ID NO:13 (AB2), (b) SEQ ID NO:21 (AB3), (c) SEQ ID NO:7 (AB1), (d) SEQ ID NO:27 (AB4) to immobilized human VEGFR-2/Fc at the concentrations of (a) 0.1, 0.2, 0.3, 0.5, 1 & 2 μM, (b) 0.2, 0.3, 0.5, 0.75, 1, 1.5, 2 & 3 μM, (c) 0.15, 0.25, 0.5, 1, 2 & 4 μM, (d) 75, 150, 225, 300, 375, 525 & 750 nM, respectively, are shown in FIG. 2.

### Example 4: Kinetic Constant Analyses of AB1 Binding to Human VEGFR-2/Fc

Derivatized data of AB1 at the concentrations of 0.1, 0.15, 0.25, 0.5, 0.75, 1, 2, & 4 μM are shown in FIG. 3. Plot for Conc. vs −ks (incept showing the concentrations below 1 μM).

$$dR / dt = \underbrace{ka C R max}_{constant} - ks R \qquad ( ks ≡ ka C + kd )$$

### Example 5: Epitope Mapping

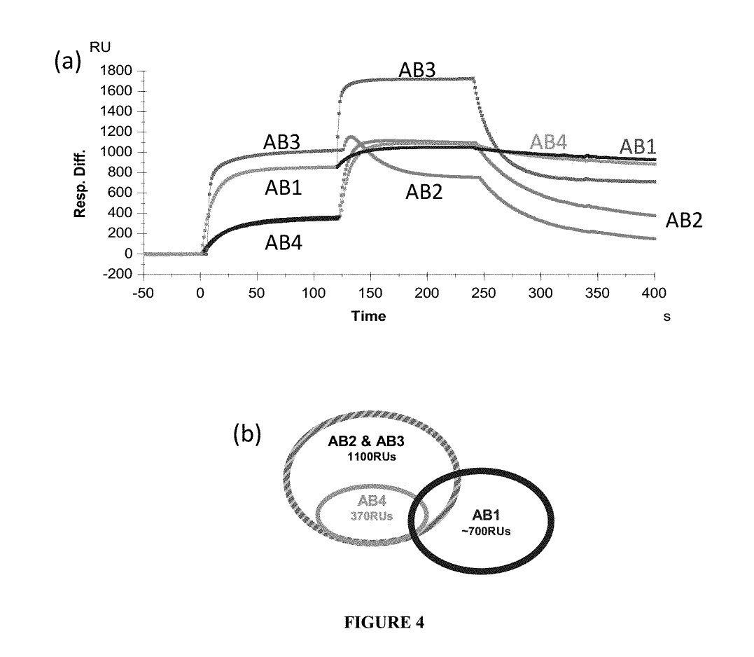

Two different antibodies were co-injected one after another at the concentrations >4×K_{D}. Results are shown in FIG. 4A and FIG. 4B. Clear overlap was seen with SEQ ID NO: 13 (AB2), SEQ ID NO:21 (AB3) and SEQ ID NO:27 (AB4). Some overlap was seen with SEQ ID NO:7 (AB1).

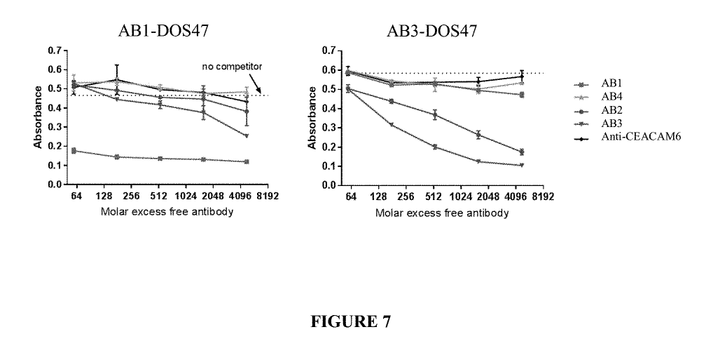

Epitope information was also provided in competitive ELISA experiments (FIG. 7). The AB3 (SEQ ID NO:23)-urease conjugate was inhibited by uncoupled AB2 (SEQ ID NO:13) antibody, suggesting that the two human antibodies share at least partially overlapping epitopes. The uncoupled AB3 (SEQ ID NO:21) antibody also partially inhibited the binding of AB1 (SEQ ID NO:9)-DOS47, although only at very high molar ratios.

### Example 6: VEGFR-2 Binding and Cross-Reactivity to VEGFR-1 and VEGFR-3

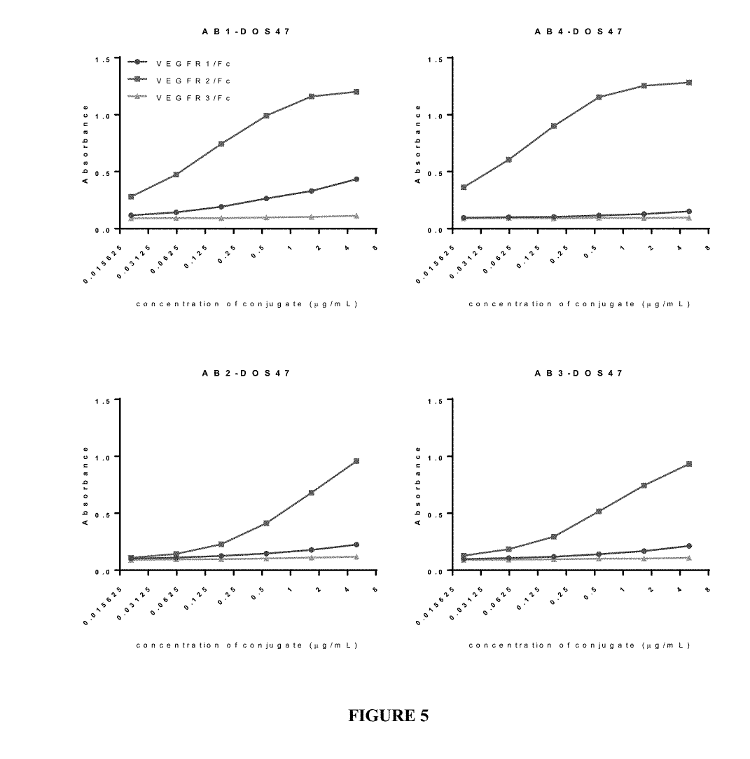

All four single domain antibodies were used to make urease (“DOS47”) conjugates. These conjugates were tested for their ability to bind the antigen VEGFR-2 and also their ability to cross-react with VEGFR-1 and VEGFR-3 (FIG. 5). All four conjugates were able to target VEGFR-2 with some cross-reactivity to VEGFR-1, but there was no detectable binding to VEGFR-3 observed. Results are shown for SEQ ID NO:9, SEQ ID NO:13, SEQ ID NO:23, and SEQ ID NO:27 (AB1, AB2, AB3, and AB4, respectively, comprising linkers).

### Example 7: VEGF Competition Assays

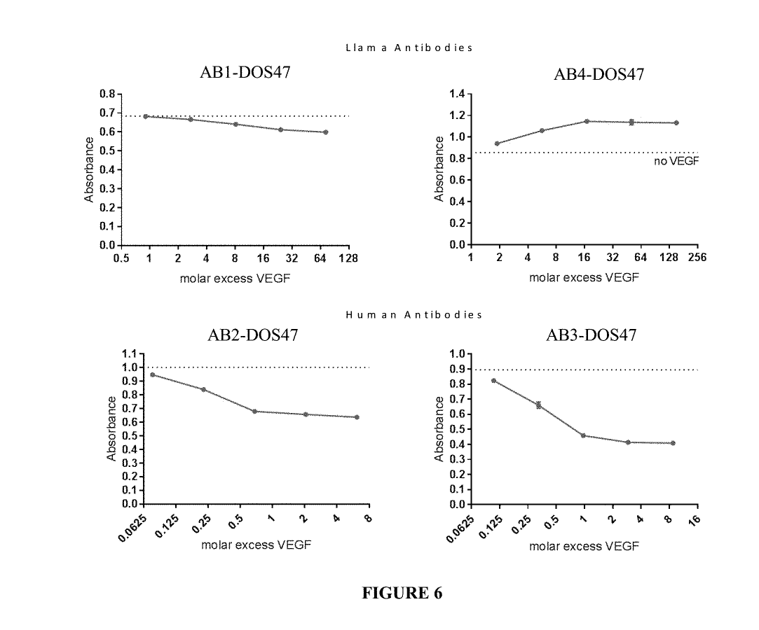

Urease conjugates were also tested for their ability to bind competitively with VEGF. This was done to assess whether the antibodies recognize a region near the VEGF binding pocket. An example of this analysis is provided in FIG. 6. From this, it can be seen that the binding of the two human antibody-urease conjugates (AB2—(SEQ ID NO:13) & AB3—(SEQ ID NO:23) DOS47) to VEGFR-2 was competitively inhibited by VEGF. However, maximum inhibition was found to be plateaued at ˜40% for AB2—(SEQ ID NO: 13) DOS47 and ˜60% for AB3—(SEQ ID NO:23) DOS47. This suggested that AB2 and AB3 only bind near the VEGF binding pocket. VEGF had a minimal effect on AB1—(SEQ ID NO:9) DOS47 complex binding to VEGFR2. Thus, it appears that AB1 binds a site remote from the VEGF binding pocket. The binding of AB4—(SEQ ID NO:27) DOS47 to VEGFR2 was enhanced by the presence of VEGF, suggesting that the AB4 antibody binds better to the VEGF/VEGFR2 complex than to VEGFR2 alone.

### Example 8: Antibody Binding to VEGFR2 Expressed on 293/KDR Cells

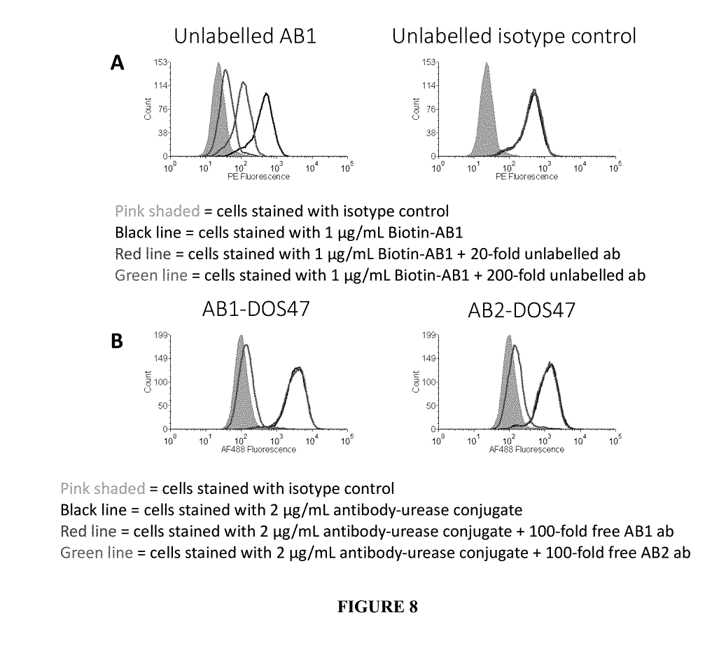

Flow cytometry experiments were performed to test the binding of antibodies and/or antibody-urease conjugates to 293/KDR cells, 293/KDR cells are 293 cells that have been stably transfected to express human VEGFR2 (also called KDR). FIG. 8A shows the binding of biotinylated AB1 antibody (SEQ ID NO:6) to 293/KDR cells. This binding is inhibited by molar excess free AB1 antibody, but not an irrelevant antibody. FIG. 8B shows the binding of the AB1—(SEQ ID NO:6) urease conjugate and the AB2—(SEQ ID NO: 18) urease conjugate to 293/KDR cells. The results shown in FIG. 8 confirm that the AB1 and AB2 antibodies described herein bind to VEGFR2 expressed on 293/KDR cells.

### Example 9

V21-DOS47 is composed of a camelid single domain anti-VEGFR2 antibody (V21) and the enzyme urease (DOS47). The conjugate specifically binds to VEGFR2 and urease converts endogenous urea into ammonia, which is toxic to tumor cells. Previously, we developed a similar antibody-urease conjugate, L-DOS47, which is currently in clinical trials for non-small cell lung cancer. Although V21-DOS47 was designed from parameters learned from the generation of L-DOS47, additional work was required to produce V21-DOS47. In this study we describe the expression and purification of two versions of the V21 antibody: V21H1 (SEQ ID NO:3) and V21H4 (SEQ ID NO:6). Each was conjugated to urease using a different chemical cross-linker. The conjugates were characterized by a panel of analytical techniques including SDS-PAGE. SEC, Western blotting, and LC-MS^{E} peptide mapping. Binding characteristics were determined by ELISA and flow cytometry assays.

To improve the stability of the conjugates at physiologic pH, the pIs of the V21 antibodies were adjusted by adding several amino acid residues to the C-terminus. For V21H4, a terminal cysteine was also added for use in the conjugation chemistry. The modified V21 antibodies were expressed in the E. coli BL21 (DE3) pT7 system. V21H1 was conjugated to urease using the heterobifunctional cross-linker succinimidyl-[(N-maleimidopropionamido)-diethyleneglycol]ester (SM(PEG)₂), which targets lysine resides in the antibody. V21H4 was conjugated to urease using the homobifunctional cross-linker, 1,8-bis(maleimido)diethylene glycol (BM(PEG)₂), which targets the cysteine added to the antibody C-terminus. V21H4-DOS47 was determined to be the superior conjugate as the antibody is easily produced and purified at high levels, and the conjugate can be efficiently generated and purified using methods easily transferrable for cGMP production. In addition, V21H4-DOS47 retains higher binding activity than V21H1-DOS47, as the native lysine residues are unmodified.

We have developed an antibody-drug conjugate (ADC) approach to suppress angiogenesis. Unlike most of the anti-angiogenic agents which interrupt the kinase signaling cascade by blocking the dimerization of VEGFR2 or by inhibiting kinase activity, our antibody-drug conjugate, V21-DOS47, kills VEGFR2-expressing cells by inducing cytotoxic activity at the target cells. Similar to our previous anti-tumor immunoconjugate, L-DOS47 (Tian et al., 2015), V21-DOS47 is composed of a camelid antibody and the enzyme urease (derived from jack beans, Canavalia ensiformis): the V21 antibody binds to VEGFR2, thus targeting the complex to VEGFR2 expressing cells, whereas the urease enzyme converts endogenous urea into ammonia in situ to induce cytotoxicity. Since VEGFR2 is not only expressed in the tumor vasculature but has also been identified on the surface of a variety of tumors (Itakura et al., 2000; Tanno et al., 2004; Guo et al., 2010), V21-DOS47 targets both VEGFR2⁺ vascular endothelial cells and VEGFR2⁺ tumor cells. The elevated local concentration of ammonia also neutralizes the acidic environment surrounding the tumor microvasculature, which is otherwise favorable to cancer cell growth (Wong et al., 2005). As urease is a plant product with no known mammalian homolog, it is likely to be immunogenic, although an auto-immune reaction is not expected. L-DOS47 is currently being tested in clinical trials and results show that anti-urease antibodies are formed, but no known severe immune toxicity is observed. The full impact of urease immunogenicity is still being studied.

One advantage of camelid antibodies is their relatively small size (approximately 15 kDa) compared to conventional immunoglobulins (approximately 150 kDa). This is particularly important when coupling antibodies to urease, as urease is a large protein with a molecular weight of 544 kDa. By using llama antibodies, multiple antibodies can be coupled to each urease molecule with a relatively minor increase in overall molecular weight. This allows for the generation of a high avidity therapeutic reagent that retains an acceptable biodistribution profile. Other benefits of camelid antibodies (De Genst et al., 2006; Maass et al., 2007; Harmsen and De Haard, 2007) are that they are easy to clone and express recombinantly (Arbabi Ghahroudi et al., 1997; Frenken et al., 2000), are generally more thermally and chemically stable than conventional IgG (van der Linden et al., 1999; Dumoulin et al., 2002), and they bind to epitopes that are not recognized by conventional antibodies (Lauwereys et al., 1998). In addition, they are not particularly immunogenic as human V_{H} and camelid V_{H}H domains share approximately 80% sequence identity (Muyldermans et al., 2001) and renal clearance is high (Cortez-Retamozo et al., 2002).

Antibody-urease conjugates are complex and large proteins: with multiple antibodies per urease, the molecular weight of the conjugate can reach 680 kDa. This provides a challenge to large-scale production. In our previous report, we described conjugation chemistry and separation procedures designed to address these challenges (Tian et al., 2015). In this study, we evaluated additional antibody production and conjugation chemistry methods to generate a novel antibody-urease conjugate, V21-DOS47.

In order to produce high affinity antibodies to VEGFR2, a llama was immunized with recombinant VEGFR2 and a V_{H}H phage display library was generated. The V21 antibody was isolated by panning this library with recombinant VEGFR2. Additional amino acid residues were added to the C-terminus of the V21 antibody in order to fulfill multiple objectives: to optimize the antibody pI, to target antibody expression to bacterial inclusion bodies, and to provide a unique target for cross-linking chemistry. In this report we describe two versions of the V21 antibody, designated V21H1 and V21H4, and the different methods used to conjugate each antibody to urease. Both antibody-urease conjugates were characterized with a variety of analytical techniques, including size exclusion chromatography (to evaluate protein purity). SDS-PAGE (to determine the average number of antibodies conjugated per urease) and ESI mass spectrometry (to identify conjugation sites on both the antibody and urease). The effects of conjugation ratio were examined, and the binding of the two conjugates with the same conjugation ratio were compared. Binding to VEGFR2 expressed at the cell surface was confirmed by flow cytometry.

### Purification of High Purity Urease (HPU)

Crude urease (Cat#U-80, 236 U/mg) was purchased from BioVectra Inc. (Charlottetown, PE Canada). Prior to use in conjugation, crude urease was purified to remove jack bean matrix protein contaminants such as canavalin and concanavalin A. One million units of crude urease were dissolved in 430 ml of high purity (HP) water at room temperature. The solution was brought to pH 5.15 with 10% (v/v) acetic acid and then centrifuged at 9000 rcf and 4° C. for 40 minutes. The urease-containing supernatant was cooled to 4° C. and fractionated by adding chilled ethanol to a final concentration of 25% (v/v) while maintaining the temperature at 0-8° C. The mixture was stirred overnight and then centrifuged at 9000 rcf and 4° C. for 40 minutes. The pellet was resuspended in 150 ml of acetate-EDTA buffer (10 mM sodium acetate, 1 mM EDTA, 1 mM TCEP, pH 6.5) and then centrifuged at 4° C. and 9000 rcf for 40 minutes. The supernatant was concentrated to 75 ml using a Minimate TFF system (Masterflex Model 7518-00 with a Minimate TFF capsule, MWCO 100 kDa), diafiltered 3 times with 200 ml of acetate-EDTA buffer, and then concentrated down to 100 ml. The diafiltered urease solution was collected, and the strained solution in the capsule and tubing connections was expelled from the system with 50 ml acetate-EDTA buffer and added to the collected solution (total volume ˜150 ml). The ethanol fractionated urease solution was further purified by anion exchange chromatography using a Bio-Rad Biologic LP system. The urease solution was loaded at a flow rate of 3.5 ml/min onto a 35 ml DEAE column (DEAE Sepharose Fast Flow, GE Healthcare, Cat#17-0709-01) which was pre-equilibrated with 150 ml of IEC Buffer A (20 mM imidazole, 1 mM TCEP, pH 6.5). The column was washed with 100 ml of IEC Buffer A, followed by 80 ml of 40% Buffer B (Buffer A with 0.180M NaCl). The urease was eluted with 100% Buffer B at a flow rate of 3.5 ml/min and fractions with A₂₈₀>0.1 were pooled. The pooled fractions were concentrated to a target protein concentration of 6-8 mg/ml using a Minimate capsule with a 100 kDa MWCO membrane and then diafiltered against acetate-EDTA buffer (20 mM sodium acetate, 1 mM EDTA, pH 6.5). The high purity urease (HPU) was stored at −80° C. The yield from this purification protocol is typically >55% of the starting activity.

### Expression of V21H1 and V21H4

Both antibodies were expressed in the E. coli BL21 (DE3) pT7 system with kanamycin as the selection antibiotic. Transformation of BL21(DE3) competent E. coli cells (Sigma, B2935-10×50 μl) was according to the manufacturer's instructions. One colony from a transformation plate was aseptically inoculated to 200 ml of LB broth (LB media EZ mix. Sigma Cat# L76581, 20 g/L) supplemented with 50 mg/L kanamycin. Cultures were incubated at 200 rpm and 37° C. Once the culture reached an OD₆₀₀ greater than 0.6, 50 ml of culture was transferred to four 2 L flasks, each containing 1L of LB broth with 50 mg/L kanamycin. Flasks were incubated in a shaker incubator at 200 rpm and 37° C. Once the culture reached an OD₆₀₀ of 0.9-1.0, antibody expression was induced by the addition of 1 mM IPTG and overnight incubation at 200 rpm and 37° C. The cells were harvested by centrifugation into aliquots, one per 2L culture.

### Purification of V21H1

The majority of the V21H1 protein was expressed in the E. coli cytosolic solution, not in the inclusion bodies. An aliquot of cell pellet was lysed in 100 ml of lysis buffer (50 mM Tris, 25 mM NaCl, pH 6.5) by sonication in an ice-water bath for 10 minutes (Misonix 3000 sonicator, tip Part#4406: each sonicating cycle: sonicating 30 seconds, cooling 4 minutes, power 8). The lysate was centrifuged at 9000 rcf and 4° C. for 30 minutes. In order to remove the most abundant bacterial matrix proteins, the supernatant was mixed with ice-cold ethanol to a final concentration of 10% (v/v) and incubated in an ice-water bath for 30 minutes, followed by centrifugation at 9000 rcf and 4° C. for 30 minutes. The supernatant was mixed with ice-cold ethanol to a final concentration of 45% (v/v) and stirred in an ice-water bath for 60 minutes, followed by centrifugation at 9000 rcf and 4° C. for 30 minutes. The pellet was resuspended in 200 ml of wash buffer (50 mM acetate, 0.1% Triton X-100, 1 mM DTT, 25 mM NaCl, pH 5.0). After centrifugation at 9000 rcf and 4° C. for 30 minutes, the pellet was resuspended in 100 ml of SP Buffer A (50 mM acetate, 8M urea, pH 4.0) supplemented with 2 mM DTT, and filtered through a 0.45 μm filter. The filtered solution was loaded on to a 1 ml SP FF column (GE Healthcare, catalog #17-5054-01) with a peristatic pump at 2 ml/minute, and the column was then connected to an ACTA FPLC system (Amersham Bioscience, UPC-920). After washing the column with 10 ml of SP Buffer A at 1 ml/min, the V21H1 antibody was eluted by a gradient of 0-50% SP Buffer B (SP Buffer A with 0.7M NaCl) over 30 minutes at a flow rate of 1 ml/min. The OD₂₈₀ of the peak fraction was determined and the concentration was calculated with an extinction coefficient of 1.967/mg/ml. DTT was added to the SP column peak fraction to a final concentration of 1 mM and the pH of the solution was adjusted to 8-8.5 with 2M Tris-Base. The refolding of the antibody was performed by adding the pH adjusted SP peak fraction drop by drop to refolding buffer (100 mM Tris, 10 μM CuSO₄, pH 8.8) and continuous stirring at 40C until the refolding was completed. The refolding process was monitored by intact protein LC-MS. After refolding, the solution was centrifuged at 9000 rcf and 4° C. for 30 minutes before loading on to a 1 ml QHP column. The column was connected to a FPLC system and washed with 10 ml of Q Buffer A (50 mM HEPES, pH 7.0) at a flow rate of 1 ml/min. The antibody was eluted by a gradient of 0-40% Q Buffer B (Q Buffer A with 0.7M NaCl) in 40 minutes at a flow rate of 1 ml/min. The peak fractions from 8 L of cell culture were pooled, concentrated to 2-4 mg/ml and dialyzed against 20 mM HEPES, pH 7.1 overnight (MWCO 5-8 kDa, volume ratio 1:50) at 4° C. The final V21H1 antibody solution was filtered through a 0.22 μm syringe filter and stored at 4° C.

### Purification of V21H4

In contrast to V21H1, the majority of the V21H4 protein was expressed in the E. coli inclusion bodies. The cell pellet from each 2 L culture was resuspended in 100 ml of lysis buffer (50 mM Tris, 25 mM NaCl, pH 6.5) and mixed with lysozyme to a final concentration of 0.2 mg/ml. The cell suspension was incubated at room temperature for 30 minutes, then lysed by sonication in an ice-water bath for 10 minutes (Misonix 3000 sonicator, tip Part#4406; each sonicating cycle: sonicating 30 seconds, cooling 4 minutes, power 8). The lysate was centrifuged at 9000 rcf and 4° C. for 30 minutes. The pellet was washed twice with 400 ml of Pellet Wash Buffer (50 mM Tris, 25 mM NaCl, pH 6.5, 1% Triton X-100, 2 mM DTT) and once with 50 mM of acetic acid containing 2 mM DTT. The pellet was resuspended in 100 ml of SP Buffer A (50 mM acetate, 8M urea, pH 4.0) supplemented with 2 mM DTT and centrifuged at 9000 rcf and 4° C. for 30 minutes. The resulting supernatant was filtered through a 0.45 μm filter and loaded on to a 5 ml SP-XL column (GE Healthcare, catalog #17-1152-01) at a flow rate of 5 ml/min. After washing the column with 50 ml of SP Buffer A, the protein was eluted by a gradient of 0-50% SP Buffer B (SP Buffer A with 0.7M NaCl) over 30 minutes at a flow rate of 5 ml/min. Peak fractions were collected when A₂₈₀>700 mU. DTT was added to the pooled SP peak fraction to a final concentration of 1.0 mM and the pH was adjusted to pH 8.6-8.7 with saturated Tris base. Refolding was initiated by mixing the SP peak fraction with refolding buffer (50 mM Tris, 2M urea, 1.0 mM DTT pH 8.6-8.7). After stirring at room temperature for 2 hours, 1.2 mM cystamine was added to the refolding mixture. Refolding continued at room temperature and was monitored by RP-HPLC (Agilent 1100 system; ZORBAX-C3 column, PN883750-909; Solvent A: 0.025% (v/v) TFA in water; Solvent B: 0.025% TFA in acetonitrile; Gradient: 20-60% B over 30 minutes at a flow rate of 0.25 ml/min. 1001l of sample was collected various time points and acidified by immediately adding 1.0 μl of neat formic acid. 30 μl of each sample was injected to the column to record the chromatogram). The resulting refolding mixture was centrifuged at 9000 rcf and 4° C. for 30 minutes before loading to a 5 ml QHP column (GE Healthcare, 17-1154-01) at a flow rate of 5 ml/min. After washing the column with 50 ml of Q Buffer A (50 mM HEPES, pH 8.7), the protein was eluted by a gradient of 0-70% Q Buffer B (Q Buffer A with 0.7M NaCl). Peak fractions with A₂₈₀>700 mU were pooled. The Q peak fractions were pooled, concentrated to 6-10 mg/ml, and buffer exchanged with 10 mM HEPES, pH 7.1. The final V21H4 antibody solution was filtered through a 0.22 μm filter and stored at 4° C.

Conjugation of V21H1 to urease

10 mg of V21H1 antibody was activated with cross-linker at an antibody to cross-linker molar ratio of 1:2.4 by adding 70.4 μl of SM(PEG)₂ (10.0 mg/ml in DMF) stock solution to the V21H1 antibody while vortexing. The reaction solution was incubated at room temperature for 90 minutes. The reaction was quenched by adding 300 mM of Tris buffer (pH 7.6) to a final concentration of 10 mM and incubating at room temperature for 10 minutes. The unconjugated, hydrolyzed and quenched cross-linker was removed with a 20 ml G25 desalting column pre-equilibrated with 50 mM Tris buffer containing 50 mM NaCl and 1 mM EDTA, pH 7.1. After removing the excess cross-linker, the desalting column fraction was pooled and a 100 μl sample was collected for intact protein mass spectrometric analysis and peptide mapping analysis to evaluate the activation sites on the V21H1 antibody. The remaining pooled fraction was chilled in an ice-water bath for 5 minutes. 20 mg of high purity urease (HPU) was thawed and incubated in another ice-water bath for 5 minutes. The chilled HPU solution was poured into the activated V21H1 antibody solution while stirring. The stirring continued in an ice-water bath for five minutes, and then the reaction solution was moved to a bench at room temperature. After the conjugation reaction solution was incubated at room temperature for 90 minutes, cysteine solution (200 mM in 300 mM Tris, pH 7-7.5) was added to a final concentration of 5 mM to quench the reaction. The reaction solution was concentrated down to approximately 4 ml by centrifugation in a 15 ml centrifuge filter (MWCO 100 kDa) at 4° C. and 2000 rcf. The resulting concentrated reaction solution was divided into three aliquots before SEC separation. The separation was performed by loading each aliquot of reaction solution to a Superose 6 100/300 GL column (GE) connected to an AKATA FPLC system. The protein was eluted by an isocratic flow at 0.5 ml/min with SEC buffer (50 mM NaCl, 0.2 mM EDTA, pH 7.2) and the major peak fractions of A₂₈₀>200 mU were pooled. The peak fractions from all three SEC separations were pooled and dialyzed against 1 L of formulation buffer (10 mM histidine, 1% (w/v) sucrose, 0.2 mM EDTA, pH7.0). The resulting conjugate solution was filtered through a 0.22 μm filter and divided into 0.8 ml aliquots. Aliquots were stored at −80° C.

### Conjugation of V21H4 to Urease

20 mg of V21H4 was mixed with TCEP (100 mM in 300 mM Tris buffer, pH 7-7.5) to a final concentration of 1.5 mM and incubated at room temperature for 60 minutes. The excess TCEP and the resulting cysteamine were removed by a 25 ml G25 desalting column using Tris-EDTA buffer (50 mM Tris, 1 mM EDTA, pH 7.1). The resulting desalting fraction was pooled in a 40 ml beaker and diluted with Tris-EDTA buffer to a total volume of 30 ml. The activation reaction was performed by quickly dispensing 0.420 ml of BM(PEG)₂ stock solution (10 mg/ml in DMF) into the V21H4 antibody solution in the beaker while stirring. After incubation at room temperature for 10 minutes, the reaction solution was transferred to a 200 ml Amicon diafiltration concentrator with a filter membrane (MWCO 5 kD) and mixed with Tris-EDTA buffer up to 100 ml. The excess cross-linker was removed by connecting the diafiltration concentrator to a 70 psi nitrogen source, and concentrated down to 20 ml while stirring. After 5 cycles of dilution and concentration, the diafiltration concentrator was detached from the nitrogen source and a 100 μl sample was collected to determine the antibody activation sites (using intact protein mass spectrometric analysis and peptide mapping analysis). Tris-EDTA buffer was added to the concentrator to dilute the solution up to the 50 ml marker. The concentrator with the activated V21H4 antibody was chilled in an ice-water bath for 10 minutes while stirring. After completely thawing at 4° C., 80 mg of HPU was incubated in another ice-water bath for 5 minutes and then poured into the activated V21H4 antibody solution in the concentrator while stirring in its ice-water bath. After stirring in the ice-water bath for 5 minutes, the concentrator with the reaction solution was moved to a lab bench and incubated at room temperature for 90 minutes. The conjugation reaction was quenched by adding cysteine (100 mM in 300 mM Tris, pH 7-7.5) to a final concentration of 5 mM. After quenching the reaction at room temperature for 5 minutes, the reaction solution was transferred to another container and the concentrator was cleaned and re-installed with a new filtration membrane (MWCO 100 kDa). The reaction solution was transferred back to the concentrator and formulation buffer (10 mM histidine, 1% (w/v) sucrose and 0.2 mM EDTA, pH 7.0) was added to the 160 ml marker. The concentrator was connected to a 10 psi nitrogen source and concentrated down to 20 ml while stirring. After the dilution-concentration cycle was repeated 4 times, the diafiltration concentrator was detached from the nitrogen source and the V21H4-DOS47 conjugate solution was transferred to a new container and diluted to 40 ml. The conjugate solution was filtered through a 0.22 μm filter and divided into 0.8 ml aliquots. The aliquots were stored at −80° C.

### Size Exclusion Chromatography (SEC)

A Waters 2695 HPLC system with a 996 PAD was employed with Empower 2 software for data acquisition and processing. Chromatograms were recorded over 210-400±4 nm with the signal at 280 nm extracted for processing. Separation was performed on a Superose 6 100/300 GL column (GE). Proteins were eluted in 10 mM phosphate, 50 mM NaCl, 0.2 mM EDTA, pH 7.2. Separation was carried out with an isocratic flow at 0.5 ml/min after injection of a certain volume of neat samples. The column temperature was kept at room temperature while the sample temperature was controlled at 5±2° C.

### SDS-PAGE

A Bio-Rad Mini Gel Protein Electrophoresis kit and a Bio-RAD Molecular Imager Gel Doc XR+ with ImageLab software were employed to analyze V21-DOS47 conjugation ratios. 10 μg of protein samples were mixed with 60 μl of protein gel loading buffer and the mixture was heated to 70C for 10 minutes. Denatured samples were loaded (10 uL/well) to a 4-20% Tris-Glycine gel (Invitrogen, REF# XP04200) and electrophoresis was performed at a constant voltage of 150V with current <40 mA until the electrophoresis front reached the gel bottom. After washing, staining and destaining, the gel image was scanned with the Gel Doc XR+ imager for analysis. SDS-PAGE was also used to calculate the average number of antibodies conjugated per urease molecule. This was determined by interrogating the intensities of the five bands in the main cluster (see Tian et al., 2015 for further details). All conjugation ratios reported are average values.

### ELISA Assays

A 96-well plate was coated with 100 μL/well of goat anti-human IgG-Fc (Sigma, 5 μg/mL in PBS) at room temperature for 6 hours and then blocked with 200 μL/well of 3% BSA/PBS at 2-8° C. overnight. After washing 2× with T-TBS (50 mM Tris, 0.15 M NaCl. pH 7.6, containing 0.05% Tween-20), 100 μL/well of VEGFR1/Fc. VEGFR2/Fc or VEGFR3/Fc (R&D Systems, 0.25 μg/mL in TB-TBS (0.1% BSA/T-TBS)) was added and the plate was incubated at room temperature for 1 hour with gentle shaking. After washing 3× with T-TBS, 100 μL/well of antibody-urease conjugate or biotinylated antibody dilutions (in TB-TBS) were added and the plate was incubated at room temperature for 2 hours with gentle shaking. For antibody-urease conjugates, plates were washed 3× with T-TBS, 100 μL/well of rabbit anti-urease (1/6,000 or 1/10,000-fold dilution in TB-TBS, Rockland) was added and the plate was incubated at room temperature for 1 hour with gentle shaking. For all samples, the plate was washed 3× with T-TBS and 100 μL/well of goat anti-rabbit-AP (1/8,000-fold dilution in TB-TBS, Sigma) was added to detect antibody-urease conjugates or streptavidin-alklaline phosphatase (0.5 μg/mL in TB-TBS, Sigma) was added to detect biotinylated antibodies, and the plate was incubated at room temperature for 1 hour with gentle shaking. After washing 3× with T-TBS, 100 μL/well of substrate (4-nitrophenyl phosphate disodium salt hexahydrate, Fluka, 1 mg/mL in diethanolamine substrate buffer, Pierce) was added to each well and incubated at room temperature for 5-15 minutes with gentle shaking. The absorbance at 405 nm (A₄₀₅) of each well was acquired by scanning the plates with a UV-Vis spectrophotometer.

### Urease Activity Assay

Urease catalyzes the hydrolysis of urea to ammonia. One unit of urease activity is defined as the amount of enzyme which liberates one micromole of ammonia per minute at 25° at pH 7.3. V21H4-DOS47 samples were diluted in sample dilution buffer (0.02M potassium phosphate containing 1 mM EDTA and 0.1% (w/v) BSA, pH 7.3). 100 μl of the diluted sample was mixed with 2.00 ml of 0.25M urea (in phosphate buffer containing 0.3M sodium phosphate and 0.5 mM EDTA. pH 7.3), and incubated at 25±0.1° C. for five minutes, then the reaction was quenched by adding 1.00m of 1.0N HCl. To determine the concentration of ammonium ion produced in the enzyme reaction solution, 100 μl of the quenched reaction solution was mixed with 2.00 ml of phenol solution (0.133M phenol containing 0.25 mM sodium nitroferricyanide) in a 15 ml testing tube. After 30 seconds, 2.50 ml of NaOH—NaOCL solution (0.14N NaOH containing 0.04% sodium hypochlorite) was added to the testing tube, mixed, and incubated at 37° C. for 15 minutes. The absorbance of the solution was determined at 638 nm with the reagent reaction solution (without sample) as the blank. The urease enzyme activity was calculated according to the following equation: U/ml=D×(A×Tc×Te)/(5×E×Sc×Se) where A=absorbance at 638 nm. Tc=total volume of color reaction (4.60 ml), Te=total volume of enzyme reaction (3.10 ml), E=molar extinction coefficient of indophenol blue per assay condition (20.10 mM⁻¹·cm⁻¹), Sc=sample volume for color reaction (0.10 ml), Se=sample volume for enzyme reaction (0.10 ml) and D=dilution time. The protein concentration of each sample was determined with a Sigma total protein kit (TP0200) following the manufacturer's instructions. Urease activity/mg of conjugate was calculated by dividing the urease activity (U/ml) by the amount of protein tested (mg/ml). Specific urease activity was calculated by dividing the activity/mg conjugate by the proportion of the conjugate's mass which was composed of urease.

### Western Blot

V21H4-DOS47 test samples and controls were resolved by SDS-PAGE gel electrophoresis and then transferred to a nitrocellulose membrane using a Bio-Rad blot kit. 1.2 μg of HPU and 4.0 μg of V21H4 as controls, and 2.0 μg of V21H4-DOS47 samples were mixed with 60.0 μl of protein gel loading buffer. The resulting sample mixtures were denatured by heating to 60° C. for 10 minutes and 10 μl of each sample was loaded per lane. Duplicate blots were made from gels run in parallel for urease and V21H4 antibody probing. For urease detection, a rabbit anti-urease IgG (Rockland) was used. To detect the V21H4 antibody, a rabbit anti-llama IgG (ImmunoReagents Inc.) was used. A goat anti-rabbit IgG conjugated to AP (Sigma) was used as the secondary visualization antibody. Final development of the Western blots was performed with AP buffer containing NBT/BCIP.

### Mass Spectrometry

A Waters Xevo G2 QTOF mass spectrometer and an Acquity UPLC system H class were employed for all mass spectrometry analyses. A lock mass of 785.8426 Da was applied for real time point to point mass calibration. LC-MS data acquisition was controlled by Masslynx V4.1 software.

### Intact Protein Mass Spectrometry Analyses

Cross-linker activated antibody samples were reacted with 5 mM cysteine at room temperature for 30 minutes, diluted to 0.5-1 mg/ml in water, and acidified by adding neat formic acid to a final concentration of 1% (v/v). A BEH300 C4 (1.7 μm, 2.1×50 mm) column was used. The column temperature was set at 60° C. and Solvent A (0.025% v/v TFA in water) and Solvent B (0.025% TFA in acetonitrile) were used for UPLC separation. The UPLC was performed with a flow rate of 0.15 ml/min with a gradient from 20 to 60% Solvent B over 30 minutes. LC-MS TIC (total ion counts) data acquisition was carried out in an M/Z range of 500-3500 Da in resolution mode with a scan rate of 0.3/s, capillary voltage 3.0 kV, sample cone voltage 40V, extraction cone voltage 4.0 kV. Ion source temperature was set at 100° C. and desolvation temperature was set at 350° C. Desolvation gas flow rate was 600 L/hour. A real time lock mass TIC raw data set (scan/20s) was acquired with 100 fmole/μl Glu-Fib B at a flow rate of 6.0 μl/min. Mass spectrometric raw data were processed with BioPharmalynx software (v1.2) in intact protein mode with a resolution of 10000. Mass match tolerance was set at 30 ppm, and the protein sequence of each antibody containing one disulfide bond was input as the match protein for protein match searches.

Tryptic Digestion of V21H1-SM(PEG)₂-Cys and V21H4-BM(PEG)₂-Cys

The cross-linker activated antibody samples were reacted with 10 mM cysteine at room temperature for 30 minutes and then diluted to 0.5 mg/ml with 100 mM ammonia hydrogen carbonate. Neat acetonitrile was added to the diluted sample solution to a final concentration of 20% (v/v). Trypsin/Lys-C Mix (Promega, Ref#V507A) was added at a protein:protease ratio of 20:1 and digested at 37C for 16-20 hours. DTT was added to the digested sample to a final concentration of 10 mM and samples were incubated at 37° C. for 30 minutes to reduce the core disulfide bond. The digestion was stopped by adding neat formic acid to 1% (v/v) before mass spectrometry analysis.

### Tryptic Digestion of V21H4-DOS47

100 μg of V21H4-DOS47 was mixed with DTT to a final concentration of 10 mM and neat acetonitrile was added to a final concentration of 20% (v/v). To reduce the disulfide bond and denature the conjugated proteins, the sample mixture was heated at 60° C. for 30 minutes. The denatured protein precipitate was pelleted by centrifugation at 16000 rcf at room temperature for 5 minutes. 5.0 μl of 0.20M iodoacetamide and 100 μl of water were added to the pellet then mixed by vortexing. The suspension was centrifuged at 16000 rcf at room temperature for 5 minutes and the supernatant was discarded. The resulting pellet was dissolved in 100 μl of Tris-guanidine buffer (4M guanidine chloride, 50 mM Tris, 10 mM CaCl₂ and 10 mM iodoacetamide, pH 8.0). After this alkylation reaction was performed at room temperature in the dark for 30 minutes, the reaction was quenched with 5 mM DTT. The resulting solution was diluted 4 times with Tris buffer (50 mM Tris, 10 mM CaCl₂ pH 8.0). Trypsin/LysC mix was added to the diluted sample solution at a protein:protease ratio of 25:1. After the digestion was performed at 37° C. for 16-20 hours, the reaction was stopped by adding neat formic acid at a final concentration of 1% (v/v).

LC-MS^{E} Peptide Mapping of V21H1-SM(PEG)₂-Cys, V21H4-BM(PEG)₂-Cys, and V21H4-DOS47 Tryptic Digests

A BEH300 C18 (1.7 μm, 2.1×150 mm) column was used for UPLC separation. The column temperature was set at 60° C. Solvent A (0.075% v/v formic acid in water) and Solvent B (0.075% formic acid in acetonitrile) were used for peptide elution. UPLC was performed with a flow rate of 0.15 mL/min. A gradient of 0 to 30% solvent B in 50 minutes was used for the separation of the tryptic digests of V21H1-SM(PEG)₂-Cys and V21H4-BM(PEG)₂-Cys samples. For the tryptic digests of V21H4-DOS47, a gradient of 0 to 45% Solvent B in 150 minutes was used. LC-MS^{E} TIC (total ion counts) data acquisitions were carried out in an M/Z range of 50-2000 Da in resolution mode with a scan rate of 0.3/s, capillary voltage 3.0 kV, sample cone voltage 25 V, and extraction cone voltage 4.0 kV. Ion source temperature was set at 100° C. and desolvation temperature was set at 350° C. Desolvation gas flow rate was 600 L/hour. A real time lock mass TIC raw data set (scan/20 s) was acquired with 100 fmole/μL Glu-Fib B at a flow rate of 3.0 μL/min. With the instrument setup, two interleaved scan functions are applied for data acquisitions. The first scan function acquires MS spectra of intact peptide ions in the sample while applying no energy to the collision cell. The second scan function acquires data over the same mass range; however, the collision energy is ramped from 20 to 60 eV. This scan is equivalent to a non-selective tandem mass spectrometric (MS/MS) scan, and allows for the collection of MS^{E} fragment spectra from the ions in the preceding scan. The high energy collision induced fragmentation randomly cleaves peptide backbone bonds. For each C—N peptide backbone bond cleaved, the amino-terminal ion generated is called the “b” ion and the C-terminal ion generated is called the “y” ion. In Tables 1-3, the column entitled “MS/MS b/y Possible” indicates the theoretical maximum number of b and y ions that would be produced for each peptide if all peptide bonds in the protein were equally likely to be broken. The column entitled “MS/MS b/y Found” indicates the actual number of b and y ions identified for each peptide. The identification of b/y ions provides unambiguous confirmation of peptide identity. Mass spectrometric raw data were processed with BiopharmaLynx software (v 1.2) in peptide map mode with a resolution of 20000. A lock mass of 785.8426 Da was applied for real time point to point mass calibration. The low energy MS ion intensity threshold was set at 3000 counts and the MS^{E} high energy ion intensity threshold was set at 300 counts. Mass match tolerances were set at 10 ppm for MS and at 20 ppm for MS^{E} data sets. Peptides with 1 missed cleavage site were included in mass match searching. V21H1, V21H4 and urease (Uniprot P07374) protein sequences were respectively input into the sequence library for peptide matching/identification. Variable modifiers including Deamidation N, Deamidation succinimide N, Oxidation M, +K, +Na, and Carbamidomethyl C (for alkylated cysteine) were applied for peptide map analysis. SM(PEG)₂-Cys (429.1206 Da) was set as a variable modifier to identify the activation sites of V21H1 conjugation, whereas BM(PEG)₂-Cys (431.1362 Da) was input as a variable modifier to identify the activation sites of V21H4 conjugation. For the V21H4-DOS47 tryptic digests, GGGEEDDGC-BM(PEG)₂ (SEQ ID NO:72) (1145.3453 Da) was set as a variable modifier to identify the conjugation sites on urease.

### Flow Cytometry

293 or 293/KDR cells were detached from flasks using non-enzymatic cell dissociation buffer (Sigma). Cells were centrifuged at 300×g for 5 minutes and then resuspended in staining buffer at 10⁶ cells/mL (PBS with Ca²⁺ and Mg²⁺, 0.02% NaN₃, 2% FBS). 100 μL of cells was added to wells of a 96-well plate. The plate was centrifuged at 350×g for 4 minutes, buffer removed, and then cells were resuspended in 50 μL of antibody-urease conjugate or biotinylated antibody (diluted in staining buffer) and then incubated at 2-8° C. for 1 hour. For cells stained with antibody-urease conjugates, cells were washed 3× with staining buffer and then resuspended in mouse anti-urease (Sigma, cat #U-4879) at 5.8 μg/mL (diluted in staining buffer) incubated for 30 minutes at 2-8° C. For all samples, cells were washed 3× with staining buffer and then resuspended in AF488-anti-mouse IgG (Jackson, cat #115-545-164) at 3 μg/mL (diluted in staining buffer) for antibody-urease samples or with PE-SA (Biolegend, cat #405204) at 133 ng/mL (diluted in staining buffer) for biotinylated antibodies. All cells were incubated for 30 minutes at 2-8° C. in the dark, washed 3× with staining buffer, then resuspended in 1% paraformaldehyde (diluted in PBS). The plate was incubated for 15 minutes at room temperature, covered with tin foil. The plate was then centrifuged as above, paraformaldehyde removed, and the cells were resuspended in staining buffer. The plate was covered in tin foil and stored at 2-8° C. until analysis using a Guava flow cytometer and guavaSoft software (Millipore). S/N values are the ratio of V21H4-DOS47 binding to 293/KDR cells vs V21H4-DOS47 binding to 293 cells or the ratio of biotin-V21H4 vs biotin-isotype control antibody (anti-CEACAM6) binding to 293/KDR cells.

### Results

### Production and Purification of V21H

When generating single-domain antibodies for immunoconjugate drugs, high purity antibodies must be produced at high yield and with controllable processes, including expression, protein refolding, and purification. Other considerations include the following: the pI of the antibody should be such that the antibody-conjugate is stable and soluble at physiologic pH, the properties of the antibody should be suitable for the conjugation chemistry, and the modifications of the antibody residues during conjugation reactions should not compromise the affinity of the antibody binding to its antigen.

The V21 camelid antibody has 122 amino acids (SEQ ID NO:2). Eleven amino acids were added to the C-terminus of the V21 antibody in order to generate V21H1 (SEQ ID NO:3). By adding these amino acids, the pI of the antibody was changed from 8.75 to 5.44, as required for conjugate stability and solubility. The hetero-bifunctional chemical cross-linker SM(PEG)₂ reacts with amine and sulfhydryl groups and was selected for use in conjugating V21H1 to urease:

### Step 1

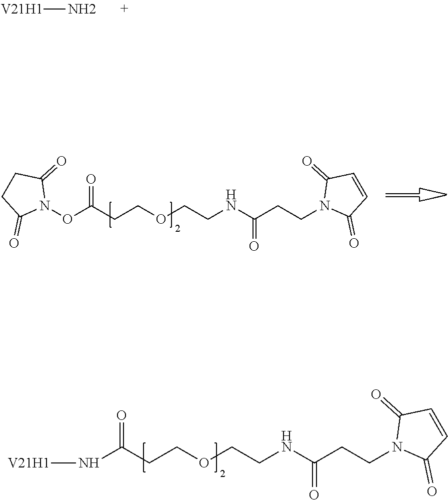

### Step 2

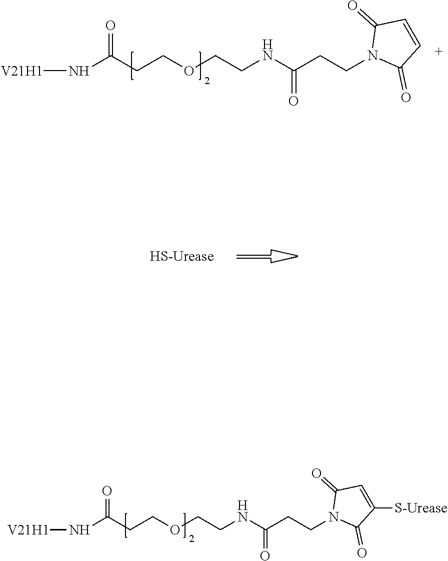

Step 1 is the activation of the antibody using SM(PEG)₂. Step 2 conjugates the activated antibody to urease.

There are five lysine residues in the core V21 sequence, two of which (Lys₆₆ and Lys₁₀₁) are located in the CDR2 and CDR3 sequences respectively. As these amino acids could be modified by the amine conjugation chemistry utilized by SM(PEG)₂, potentially altering antibody activity, two extra lysine residues were added to the antibody C-terminus to minimize this probability.

V21H1 was expressed primarily in the cytosolic solution of BL21(DE3) bacteria, with virtually no expression in inclusion bodies. Therefore, after cell lysis, the antibody was separated from bacterial proteins by ethanol crystallization and cation-exchange chromatography. After antibody refolding, the native antibody was further purified by anion-exchange chromatography. To confirm that the molecular mass of the purified antibody matched the designed protein sequences, LC-MS intact protein analysis was performed. No impurity proteins were detected from the LC-MS TIC chromatograms and the detected molecular mass of V21H1 matched the theoretical value calculated from its protein sequence within 30 ppm mass match error (data not shown). However, the yield of purified V21H1 was very low (4-6 mg/L of culture) and the purification processes used are not suitable for large scale cGMP production.

### Cross-Linker Activation of V21H1

V21H1 was activated by SM(PEG)₂ at pH 7.0 using conditions previously found to be optimal for activation of AFAIKL2 antibody with SIAB in the production of the antibody-urease conjugate L-DOS47. Since the NHS-ester reaction is the same for SIAB and SM(PEG)₂ and the LC-MS spectra are similar for AFAIKL2 and V21H1 reaction products (data not shown), these conditions should also be optimal for activation of V21H1 with SM(PEG)₂.

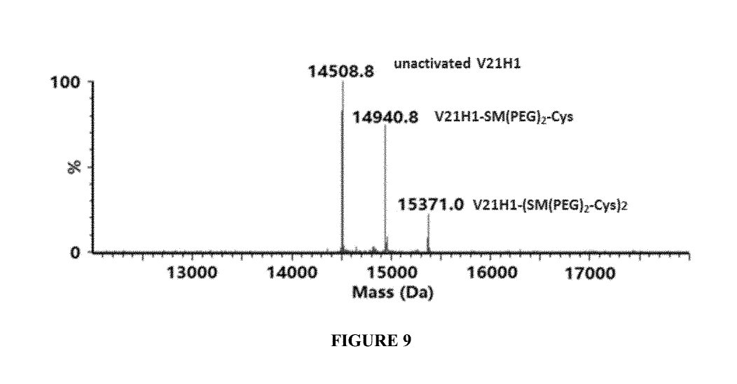

Only the NHS-ester group of SM(PEG)₂ can react with V21H1. The two cysteine residues in the V21H1 antibody form a disulfide bond and are thus unavailable to react with the maleimido end of the cross linker. The primary amines from the antibody N-terminus and the lysine residues from the protein sequence can all potentially react with the NHS-ester of the cross-linker. The maleimido end of the antibody-carrying cross-linker then reacts with cysteines on the surface of urease molecules. The probability of each amine being activated depends on its accessibility due to its surrounding native structure. To avoid urease dimer and polymers forming in the second reaction step, ideally only one amine per antibody would be activated by the NHS-ester. However, since multiple primary amines are present in each antibody, it is statistically inevitable that some V21H1 antibodies will be activated by more than one cross-linker molecule. The optimal activation condition was selected, which minimizes the percentage of antibodies that are activated by more than one cross-linker while maximizing the total amount of activated antibody. To assess the activation distribution, the SM(PEG)₂ activated V21H1 was reacted with excess cysteine and evaluated by intact mass spectrometric analysis. The mass spectrum is shown in FIG. 9. Approximately 50% of the V21H1 was activated by SM(PEG)₂ and of the activated antibody, approximately 30% was activated by two cross-linkers. Thus, only 35% of the V21H1 antibody is optimally activated for cross-linking with urease.

In order to determine which lysines of V21H1 were targeted by SM(PEG)₂, V21H1-SM(PEG)₂-Cys was subjected to tryptic digestion followed by LC-MS^{E} analysis. Trypsin cleaves peptide backbone bonds at the C-terminal side of arginine and lysine residues (unless proline is immediately C-terminal to K or R). If a lysine is activated by SM(PEG)₂, the polarity and side-chain structure of the lysine is altered and spatially blocked. Thus, this tryptic site is no longer accessible to the protease. For example, if K₆₆ of V21H1 is activated by SM(PEG)₂, it is linked to -SM(PEG)₂-Cys and is no longer be available for tryptic digestion; therefore, a peak with a molecular mass of 2862.3018 (2431.1656+431.1362) Da should be observed, which represents the -SM(PEG)₂-Cys linked lysine-in-middle peptide, (ELVAAISWSDDSTYYANSVK₆₆GR)-SM(PEG)₂-Cys. In the LC-MS^{E} peptide mapping analysis, all possible activation sites can be identified by searching all the lysine carrying peptides and the N-terminal peptide with the -SM(PEG)₂-Cys (431.1362 Da) as a variable modifier. The detected tryptic peptides along with conjugation sites are listed in Table 2.

All tryptic peptides were detected with mass match errors of less than 5 ppm and the amino acid sequence recovery was 100%. Assuming that ESI sensitivity is not affected by the linkage of the modifier, an activation percentage was assessed by comparing the intensity of the cross-linker modified peptide with the sum intensity of all the related peptides. Under the activation conditions used, lysine residue K₆₆ in CDR2 was substantially (˜25% of the entire activated V21H1 antibody) activated by the cross-linker, however, K₁₀₁ in CDR3 was not modified during cross-linker activation. Surprisingly, the two C-terminal lysine residues that were intentionally added for conjugation chemistry purposes were not modified by the cross-linker.

### Production and Purification of V21H4

The antibody V21H4 was designed to improve upon the issues identified during production, purification and cross-linker activation of V21H1. The amino acid sequence of the V21H4 antibody is shown in SEQ ID NO:6. As for V21H1, a number of amino acid residues were added to the V21 antibody C-terminus (G₁₂₃-C₁₃₆) and the pI of the antibody was adjusted from 8.75 to 5.43. With V21H1, the presence of SM(PEG)₂ cross-linker activated K₆₆ in the antibody CDR2 region was a concern as this could impair antibody binding affinity. Thus, a cysteine residue (C₁₃₆) was added to V21H4 for sulfhydryl-to-sulfhydryl crosslinking using a different cross-linker, BM(PEG)₂:

### Step 1

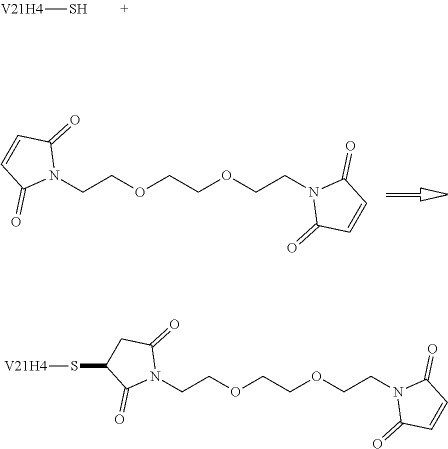

### Step 2

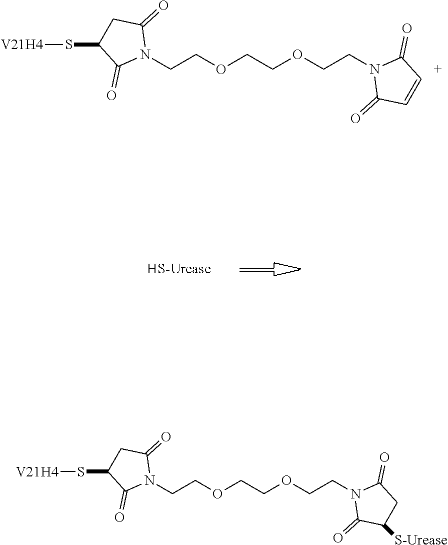

Step 1 is the activation of the antibody using BM(PEG)₂. Step 2 conjugates the activated antibody to urease.

The inclusion of a C-terminal cysteine also allowed the antibody to be expressed in bacterial inclusion bodies. As the two core cysteine residues of the V21 antibody form a disulfide bond and are unavailable for chemical conjugation, the additional C-terminal cysteine residue provides a unique activation site for targeted conjugation.

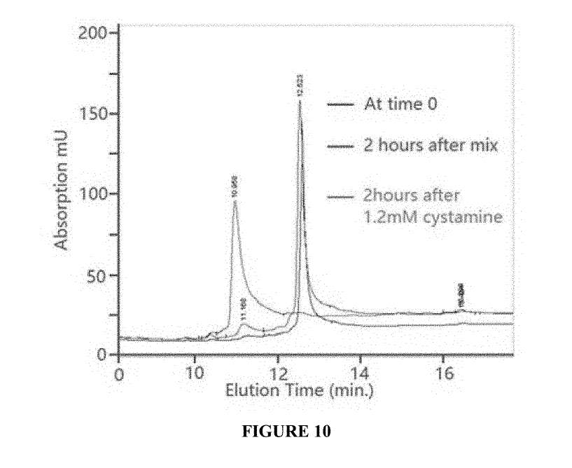

V21H4 was expressed at high levels in inclusion bodies. After cell lysis, antibody was separated from bacterial matrix proteins by centrifugation. The denatured antibody was purified by cation exchange chromatography to remove nucleic acids and other proteins. The refolding of the V21H4 antibody was performed in an easily controllable manner and was monitored by HPLC (FIG. 10).

The refolding process was initiated by mixing the peak fraction of the cation exchange column with refolding buffer. While the folding process was very slow without cystamine, folding was complete in two hours at room temperature after cystamine was added to a final concentration of 1.2 mM. Anion exchange chromatography was used to isolate the properly folded protein, and yields of greater than 80% were generally observed. The typical yield of purified V21H4 is 20-40 mg/L culture, which is considerably higher than that of V21H1. In addition, the method used to produce and purify V21H4 is amenable to scale up and cGMP procedures.

### Cross-Linker Activation of V21H4

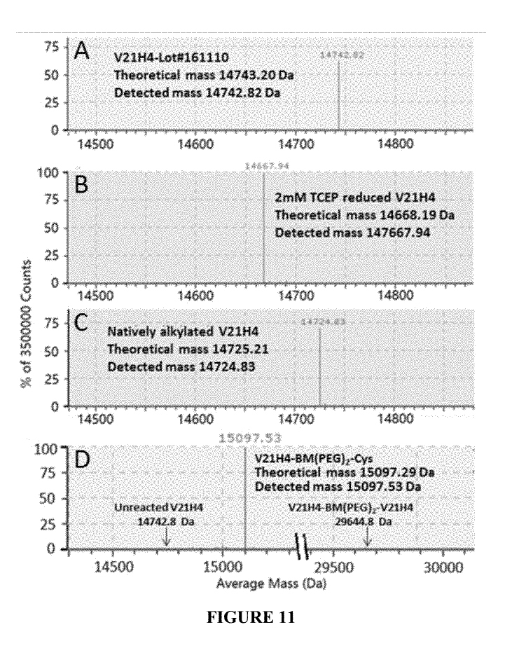

The C-terminal cysteine of V21H4 is required for conjugation to urease. However, as cystamine was included in the V21H4 refolding buffer, the C-terminal cysteine was modified by forming a disulfide bond with a half cystamine (cysteamine-H). This was confirmed by LC-MS intact protein analysis (FIG. 11A). Thus, the half cystamine must be removed and the cysteine must subsequently be available for activation by cross-linker. In addition, this removal must occur using a controllable mild reduction under the native conditions to be used for conjugation purposes and it must not reduce the antibody's internal disulfide bond. As shown in FIG. 11B, after reducing V21H4 with 2 mM TCEP at pH 7.1 for one hour at room temperature, the detected antibody molecular mass was 14667.94 Da, suggesting that the protective half cystamine had been removed. In order to confirm that the de-protected cysteine residue was active to the cross-linking reagent, 10 mM iodoacetamide was added to the de-protected V21H4 antibody. After 30 minutes at room temperature at pH 7.5-8.0, the resulting detected molecular mass was increased to 14724.83 Da (FIG. 11C), suggesting a carboxymethyl group (57.05 Da) was alkylated to the cysteine residue. In summary, the C-terminal half cystamine can be removed and the resulting de-protected cysteine is available for chemical conjugation. The alkylated antibody was also digested with trypsin and evaluated by LC-MS^{E} peptide mapping. The LC-MS^{E} peptide map (data not shown) covered 100% of the amino acid sequence and the C-terminal cysteine was specifically and effectively alkylated, confirming the specificity of the de-protective reduction reaction and the suitability of the C-terminal cysteine in targeted sulfhydryl cross-linking chemistry.

The V21H4 antibody was activated by the cross-linker BM(PEG)₂. As BM(PEG)₂ is a homo-bifunctional cross-linker, it is possible that both maleimido groups of BM(PEG)₂ could react with and link two V21H4 molecules, leading to the generation of antibody dimers that cannot conjugate to urease. The frequency of antibody dimers generated depends upon the molar ratio of the reactants, the native hydrophobicity environment of the cysteine residue and the relative mobility of the molecules in the reaction solution. This reaction was performed with a 10:1 cross-linker to antibody molar ratio. In addition, the molecular weight of the cross-linker is 308.29 Da, which is approximately 50-fold less than the molecular weight of the antibody. To evaluate the activated V21H4 antibody, 100 μl of the activated antibody solution was reacted with excess cysteine and evaluated by intact mass spectrometric analysis (FIG. 11D). Under the experimental conditions used, more than 99% of the V21H4 was coupled to a single cross-linker, leaving the cross-linker's other maleimido group available for the subsequent reaction to urease.

In order to confirm that the C-terminal cysteine was the sole target of BM(PEG)₂, V21H4-BM(PEG)₂-Cys was subjected to tryptic digestion followed by LC-MS^{E} analysis. If the C-terminal cysteine is activated by the cross-linker, a peak with a mass of 1266.3652 Da representing the cross-linker activated peptide GGGEEDDGC₁₃₆-BM(PEG)₂-Cys (SEQ ID NO:73) should be detected. If the core disulfide bond is reduced by TCEP before cross-linker activation, then two peaks—one representing the peptide LSC₂₃AASGR-BM(PEG)₂-Cys (SEQ ID NO:74) (1192.4852 Da) and the other representing SAVYLQMNSLKPEDTAVYYC₉₇-AAHK-BM(PEG)₂-Cys (SEQ ID NO:75) (3130.4087 Da) should be identified. The detected tryptic peptides along with the cross-linker activation sites are listed in Table 3.

All tryptic peptides were detected with mass match errors of less than 5 ppm, and the amino acid sequence recovery was 100%. As expected, more than 90% of the C-terminal cysteine was activated by the cross-linker, and only trace amounts of cross-linker activated core cysteine residues (Cys₂₃ and Cys₉₇) were detected. This is a much more desirable scenario than that observed with V21H1 and SM(PEG)₂, in which multiple lysines are targeted, including the one in CDR2.

### Conjugation of V21H1 and V21H4 to Urease and Initial Characterization

Jack bean urease is a homohexameric enzyme with each subunit approximately 91 kDa. Among the 15 unbound cysteine residues per subunit, five are on the surface of the native structure and are available for linking to single-domain antibodies through maleimido cross-linkers (Takishima et al., 1998). Different conjugation chemistries are widely used for protein conjugations. Copper-free click chemistry has been preferentially used in protein labeling and protein-drug conjugations (Thirumurugan et al., 2013) and was a potential option in our conjugations of antibodies to urease. However, either the NHS-ester or maleimido activation step would be needed before performing the click chemistry. Thus, traditional cross-linking chemistries are simpler and are suitable to this particular case.

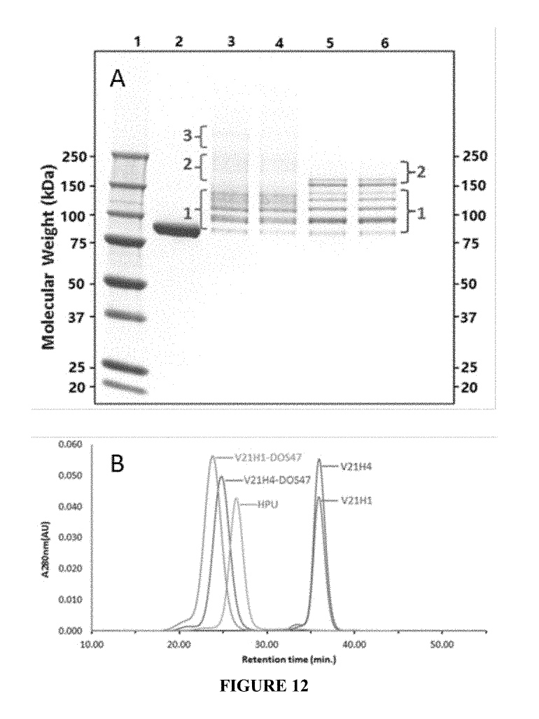

After V21H1 and V21H4 were cross-linked, they were then conjugated to urease to generate V21H1-DOS47 and V21H4-DOS47, respectively. In both cases, sulthydryl chemistry was used to conjugate the antibody-linker to urease. SDS-PAGE was performed to evaluate both conjugates (FIG. 12A).

During conjugation, each of the six monomeric urease subunits could potentially be cross-linked with up to five antibody molecules: therefore, under denaturing SDS-PAGE conditions, both V21H1-DOS47 and V21H4-DOS47 would be expected to generate a pattern of six discrete bands ranging from ˜90-180 kDa. However, it appears that a maximum of four antibodies are conjugated per urease, as only five discrete bands are observed (FIG. 12A, cluster 1). This suggests that one of the five cysteine residues on the surface of urease has little or no ability to react with maleimide.

In addition to the expected five discrete bands, additional clusters of bands are observed for both V21H1-DOS47 and V21H4-DOS47. For V21H1-DOS47, two additional clusters are apparent. Cluster 2 (effective MW from ˜200 to 250 Da) and cluster 3 (effective MW>300 Da) are likely urease dimers and polymers generated by V21H1 species carrying multiple SM(PEG)₂ cross-linkers. While these higher molecular weight species could be composed of multiple native urease molecules, the low levels (less than 5%) of dimer and polymer peaks observed by size exclusion chromatography (FIG. 12B) suggests that the majority of these species are composed of inter-subunit linkages of a single native urease molecule and not inter-molecular linkages.

For V21H4-DOS47, since only the C-terminal cysteine is activated by BM(PEG)₂, theoretically only one band cluster should be present. However, as demonstrated in Lanes 5 and 6, an additional cluster is observed in the V21H4-DOS47 lanes (MW≥than 150 kDa). The second cluster could be composed of non-covalent dimers that form as the conjugated subunits migrate in the gel. This was confirmed by SDS-PAGE capillary electrophoresis (not shown) in which no dimer clusters were observed. Therefore, V21H4-DOS47 does not contain cross-linked urease dimers or polymers.

SDS-PAGE was also used to determine the antibody:urease conjugation ratio for each native urease hexamer-antibody conjugate. Band intensities (FIG. 12A) in cluster 1 depend upon the relative abundance of urease monomers linked to different numbers of antibody molecules. ImageLab software was used to generate histograms corresponding to band intensities and to integrate the peak areas of each histogram. The conjugation ratio (CR) for native urease hexamers was calculated as follows:

CR=6*(PK₁*0+PK₂*1+PK₃*2+PK₄*3+PK₅*4)/(PK₁+PK₂+PK₃+PK₄+PK₅)

Where PKᵢ (i=1-5) is the peak area of the urease monomer linked with i-1 antibody molecules.

Although there is a variable number of antibodies conjugated to each urease monomer, one would predict less variability in the number of antibodies per urease hexamer, as the monomers randomly cluster to form hexamers. This was confirmed by SEC of native V21H4-DOS47 in which the conjugate is observed to migrate as a tight peak (FIG. 12B). The V21H4-DOS47 conjugation method reproducibly produced conjugates with 8.7-9.2 antibodies per urease (based on three batches).

The purities and the effective molecular weights of the antibodies, HP urease, and conjugates were assessed by size exclusion chromatography (SEC) under native conditions (FIG. 12B).

V21H1 and V21H4 antibodies elute at comparable times (35.9 minutes). Free HP urease elutes at 26 minutes. As antibody molecules are linked to urease molecules for both V21H1-DOS47 and V21H4-DOS47, making the conjugates larger than free urease, the conjugates elute earlier than free urease. However, it is interesting that V21H1-DOS47 elutes one minute before V21H4-DOS47 (22.80 vs 23.80 minutes). Both conjugates have nearly identical conjugation ratios (8.8 antibodies/urease for V21H1-DOS47 and 8.7 antibodies/urease for V21H4-DOS47). The V21H4 antibody has three more amino acids (159.20 Da) than V21H1; however, the theoretically larger V21H4-DOS47 conjugate appears smaller in effective molecular size in SEC than its counterpart V21H1-DOS47. This implies that V21H4-DOS47 is more compact than V21H1-DOS47 under native conditions.

The majority of each species is in the monomeric form, with small dimer peaks appearing in front of each monomeric peak. It is notable that the V21H1-DOS47 conjugation procedure requires a SEC step in order to achieve high purity (96%). The SEC step removes urease polymers that are generated by V21H1 antibodies activated by two cross-linkers. However, the SEC step is not necessary to produce V21H4-DOS47, as V21H4 antibodies are activated by one cross-linker only. For V21H4-DOS47, a purity of greater than 97% is typically achieved using only diafiltration to remove unbound V21H4 antibody. As SEC methods are not easily transferred to large-scale GMP processes, it would be technically more difficult and expensive to produce V21H1-DOS47 for clinical use.

### Activity of V21H1-DOS47 and V21H4-DOS47

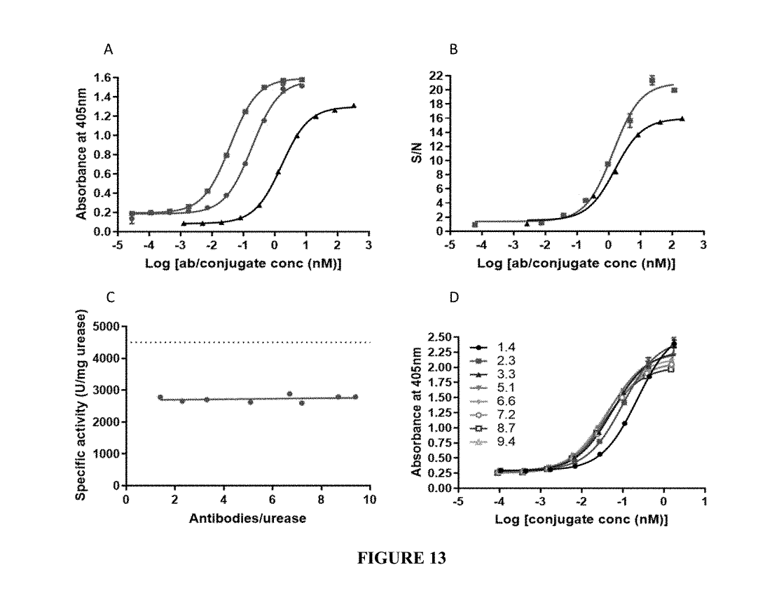

An ELISA assay was performed to evaluate the binding of V21H1-DOS47 (9.2 antibodies/urease), V21H4-DOS47 (8.8 antibodies/urease) and biotin-V21H4 to recombinant VEGFR2/Fc (FIG. 13A). V21H4-DOS47 (EC₅₀=44 μM) binds to VEGFR2/Fc with approximately five-fold higher affinity than does V21H1-DOS47 (EC₅₀=226 μM). As a substantial amount of V21H1 was conjugated to urease via the lysine present in CDR2, this is not surprising. V21H4-DOS47 also binds to VEGFR2/Fc with approximately 40-fold higher affinity than does V21H4 antibody alone (EC₅₀=1.8 nM). This is most likely due to the multivalent nature of the conjugate. As V21H4-DOS47 is the superior conjugate, subsequent characterization was performed for V21H4-DOS47 only.

The ability of V21H4 antibody and V21H4-DOS47 conjugate to bind to cells expressing VEGFR2 (293/KDR) was evaluated by flow cytometry (FIG. 13B). Biotin-V21H4 (EC₅₀=1.6 nM) binds to 293/KDR cells with a similar affinity as to recombinant VEGFR/Fc (EC₅₀=1.8 nM, FIG. 13A). This suggests that the VEGFR2 antibody epitope is equally accessible in recombinant VEGFR2/Fc in the ELISA assay and on the cell surface of 293/KDR cells. Interestingly, the binding of V21H4-DOS47 (EC₅₀=1.2 nM) to the 293/KDR cells is very similar to the binding of biotin-V21H4 antibody to these cells (EC₅₀=1.6 nM). Although there was an improved affinity observed for V21H4-DOS47 compared to V21H4 antibody in the ELISA assay with VEGFR2/Fc, this was not observed for cell binding. This suggests that the density of VEGFR2 expressed on the surface of 293/KDR cells is lower than in the wells of the ELISA plate.

Several factors contribute to determination of an ideal antibody/urease conjugation ratio. During the conjugation reaction, the urease molecule is altered by linkage to the V21 antibody; therefore, depending on the conjugation ratio, urease enzyme activity could be affected. On the other hand, the avidity of the antibody-urease complex increases as more antibodies are coupled to urease. To evaluate the effects of conjugation ratio on both the urease enzyme activity and on binding activity, V21H4-DOS47 conjugates with different conjugation ratios (1.4 to 9.4 V21H4 per urease) were produced by adjusting the V21H4/HPU molar ratios.

The activity of unmodified urease is approximately 4500 U/mg. When antibody is conjugated to urease, approximately 40% of the activity is lost (FIG. 13C). However, the urease enzyme activity is independent of the number of antibodies conjugated, as activity remains consistent at all conjugation ratios tested. An ELISA assay using recombinant VEGFR2/Fc was performed to evaluate the binding of conjugates with different numbers of antibodies per urease (FIG. 13D). When increasing from 1.4 to 2.3 antibodies per urease, the binding of the conjugate to VEGFR2/Fc improves, as indicated by a decrease in EC₅₀ values from 226 μM to 93 μM. Addition of one more antibody (3.3 antibodies/urease) further reduces the EC₅₀ to 58 μM However, addition of subsequent antibodies/urease has a limited benefit: with 9.4 antibodies per urease, the EC₅₀ is 31 μM. Thus, there is only a slight improvement in affinity when greater than 3.3 antibodies per urease are present. Thus, a conjugation ratio of 3.3 antibodies per urease is sufficient for optimal urease activity and conjugate binding.

### Additional Characterization of V21H4-DOS47

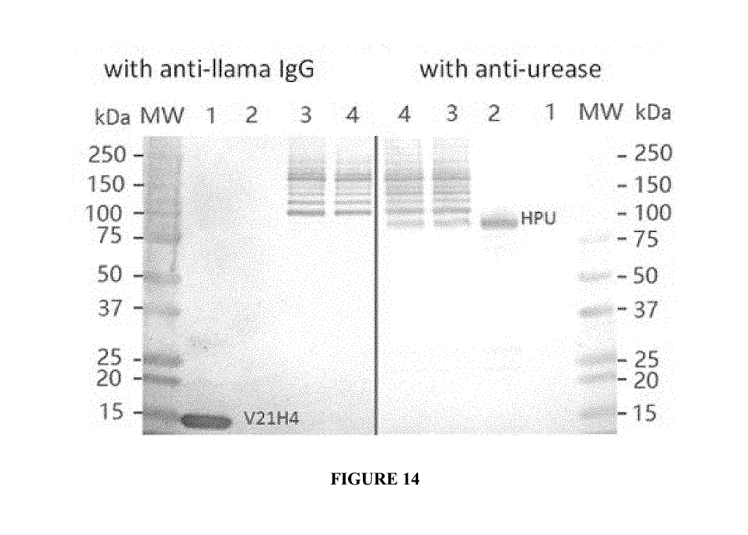

Dual-panel Western blotting (FIG. 14) of V21H4-DOS47 was performed to confirm the banding pattern seen by SDS-PAGE. In Western blotting, the dimer and polymer clusters formed in-gel are more prominent than they appeared in SDS-PAGE (FIG. 12A). When probed with anti-urease antibody, the urease band is visualized at molecular weight ˜85 kDa, and the bands of urease subunits bound to 1 to 4 antibodies match with the pattern seen by SDS-PAGE. When probed with an anti-llama antibody, the free urease subunit band is not observed and the antibody-urease conjugate bands are seen in the same pattern as when probed with an anti-urease antibody. The ability of V21H4-DOS47 to be visualized by both the anti-llama and anti-urease antibodies demonstrates the presence of both species in the conjugate.

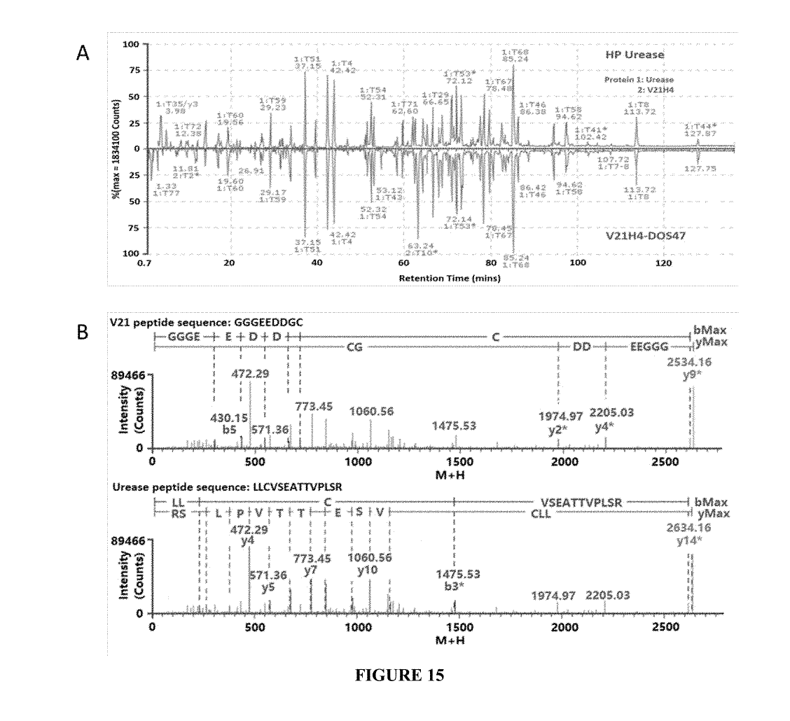

ESI-LC-MS^{E} peptide mapping analysis was employed to confirm the identities of V21H4 and urease and to identify the conjugation sites of V21H4-DOS47. The LC-MS (TIC) chromatograms of V21H4-DOS47 and HPU are shown in FIG. 15A.

The identified peptides covered 100% of V21H4 and urease protein sequences with mass match errors less than 4 ppm. All identified peptides with greater than three residues were confirmed by elevated energy MS/MS with at least half of the b/y ions identified. Since only the C-terminal GGGEEDDGC (SEQ ID NO:76) (837.2446 Da) of V21H4 is linked to different cysteine-carrying peptides of urease, the conjugation sites (denoted as UCₓ-VC₁₃₆, where x is the amino acid in the urease protein sequence) are those urease peptides modified by GGGEEDDGC-BM(PEG)₂ (SEQ ID NO:72) (1145.3453 Da). To identify those covalent conjugation sites, ESI LC-MS^{E} raw data of the tryptic digests from V21H4-DOS47 samples were processed by BiopharmaLynx and searched against the urease protein sequence with a variable modifier of 1145.3453 Da applied to all 15 urease cysteine residues. In order to assess the relative frequency of each conjugation site, the peptide intensities of the conjugated peptides UCₓ-VC₁₃₆ were compared with the sum intensities of all the peptides related to UCₓ to generate the % of conjugation (Table 4).

TABLE 4
ESI LC-MS^{E} peptide mapping analysis. Identification of urease cysteine residues
modified by V21H4-(PEG)₂-Cys. na = not applicable.
Conjugation sites searched from the urease side

| | | | | | | Mass | |
|---|---|---|---|---|---|---|---|
| | | | MS/MS | MS/MS | | match | |
| Urease | Conjugation | Calculated | b/y | b/y | | error | % of |
| peptide | Site | Mass (Da) | Possible | Found | Intensity | ppm | conjugation |
| 1:T010* | UC₅₉-VC₁₃₆ | 2784.2053 | 28 | 10 | 335045 | 2.6 | 2.6 |
| 1:T026* | UC₂₀₇-VC₁₃₆ | 1939.6624 | 12 | 0 | 10296 | 1.9 | 0.6 |
| 1:T063* | UC₆₆₃-VC₁₃₆ | 2316.7554 | 18 | 4 | 46812 | 2.9 | 4.2 |
| 1:T081* | UC₈₂₄-VC₁₃₆ | 2633.1372 | 26 | 13 | 495879 | 2.1 | 26.7 |

Conjugation sites searched from the antibody side

| | | | | | | Mass | |
|---|---|---|---|---|---|---|---|
| V21H4 | | | MS/MS | MS/MS | | match | |
| C-term | Conjugation | Calculated | b/y | b/y | | error | % of |
| peptide | Site | Mass (Da) | Possible | Found | Intensity | ppm | conjugation |
| 2:T012 | na | 837.2446 | 16 | 2 | 10403 | −3.9 | 0.4 |
| 2:T012* | -UC₈₂₄ | 2633.1472 | 16 | 7 | 1609854 | 1.2 | 59.1 |
| 2:T012* | -UC₆₆₃ | 2784.2153 | 16 | 5 | 726682 | 1.6 | 26.7 |
| 2:T012* | -UC₅₉ | 2316.7654 | 16 | 4 | 343529 | −1.4 | 12.6 |
| 2:T012* | -UC₂₀₇ | 1939.6724 | 16 | 0 | 33038 | −3.6 | 1.2 |

Among the 15 cysteine residues of each urease subunit only 4 were conjugated (consistent with bands observed by SDS-PAGE, FIG. 12A). The most accessible cysteine is C₈₂₄ (26.7%), followed in order by C₆₆₃ (4.2%), C₅₉ (2.6%), and C₂₀₇ (0.6%). No conjugation was detected to cysteine residue C₅₉₂, which is essential to urease enzyme activity. This is consistent with the observation that urease activity is comparable at all conjugation ratios (FIG. 13B).

Conjugation sites were also identified as V21H4 peptides modified by −UCₓ (UCₓ+308.1008 Da). This was done by searching the V21H4 antibody protein sequence against −UCₓ as the variable modifier to the C-terminal cysteine of V21H4 (Table 3). Among the identified tryptic peptides, 0.4% of them were unmodified (T:012). This trace amount of peptide could be the portion of V21H4 activated by the cross-linker through C₂₃ and C₉₇ of the core sequence. Alternately, this peptide could be a trace amount of V21H4 attached to the C-terminal half cystamine that was not deprotected in the TCEP reduction step. These results are consistent with those observed with urease peptides modified by −VC₁₃₆. Most of the V21H4 C-terminal cysteine was conjugated to urease via C₈₂₄ (59%), with less conjugation at C₆₆₃ (27%), C₅₉ (12%), and C₂₀₇ (1.2%).

The identities of the conjugation sites were confirmed with b/v ion mapping of urease and V21H4 peptides. Among the 16 possible V21H4 b/v ions, only a few (4-7) were identified from the three major urease conjugation sites. This could be a result of the ESI ionization property of the GGGEEDDGC (SEQ ID NO:76) residues, which causes a lack of positive charge center in the ionization environment. However, the MS/MS b/y fragment profiles (FIG. 15B) can be assessed by looking at both V21H4 and urease proteins. As an example, the conjugated peptide UC₆₆₃-VC₁₃₃ whose sequence is (LLCVSEATVPLSR)-linkage-(GGGEEDDGC) and which has a peptide mass of 2633.1472 was identified with a mass match error of 2.1 ppm by searching it as LLCVSEATTVPLSR (SEQ ID NO:77), a urease peptide modified with (GGGEEDDGC)-linkage (1145.3453 Da) from the V21H4 side as the modifier. The same peptide was also identified with a mass match error of 2.1 ppm by searching it as GGGEEDDGC, a V21H4 C-terminal peptide modified with (LLCVSEATTVPLSR)-linkage (1795.9026 Da) from the urease side as the modifier. The MS^{E} collision induced MS/MS spectrum of this conjugated peptide was mapped with 13 b/y fragment ions from the urease side by searching it as a urease peptide modified with the modifier from the V21H4 side. The same spectrum was also mapped with 7 b/y ions from the V21H4 side by searching it as a V21H4 peptide with the modifier from the urease side.

### Discussion

Antibody drug conjugates are emerging as a promising class of anti-cancer drugs. By delivering drugs directly to the target site, non-specific side effects are reduced. We have previously described the production and characterization of L-DOS47, an ADC composed of the enzyme urease and an anti-CEACAM6 antibody (Tian et al., 2015). L-DOS47 is currently in phase I/II trials for the treatment of non-small cell lung cancer. In this study, the conjugate V21H4-DOS47 was generated and characterized, which targets VEGFR2. Although L-DOS47 and V21H4-DOS47 were both generated by conjugating urease to a llama antibody, considerable research was required to produce a successful V21H4-DOS47 conjugate. For example, initial V21-DOS47 conjugates generated using the same linker as in L-DOS47, SIAB, was not as successful (SAB is a short and rigid linker) as using PEG₂ class of linkers, which are relatively long and flexible, and now it is herein demonstrated that the binding activity of the conjugate was considerably improved.

In this study we developed procedures to conjugate and purify the V21-DOS47 immunoconjugate that are suitable for large scale cGMP production. Single domain camelid antibodies are ideal for use in generating antibody-enzyme conjugates. Their small molecular size allows them to be produced affordably in large amounts. Importantly, they were presently be modified by adding a short amino acid tag at the C-terminus. The tag serves several purposes, including modification of the antibody pI, promotion of targeted antibody expression, and addition of a specific reaction site. Since the pI of urease is in the 4.8 to 5.1 range, an antibody-urease conjugate generated with the unmodified core antibody would produce a conjugate with a pI of approximately 7. At this pI, the conjugate is unstable and forms precipitates during and after conjugation. The addition of a short C-terminal peptide tag adjusts the pI of the antibody from 8.75 to 5.43 leading to a conjugate with a pI between 4.8 and 5.5 which is stable during conjugation and purification. The C-terminal tag also improves the yield of antibody production by targeting expression to bacterial inclusion bodies. This allowed antibody purification using only ion exchange chromatography. As the V21 sequence contains two lysine residues in the CDR2 and CDR3 sequences respectively, lysine-to-sulfhydryl cross-linking chemistry could modify these lysine residues, compromising the binding affinity of the conjugate to its target antigen. For this reason, a C-terminal cysteine residue was included in the C-terminal tag of V21H4 for use in sulfhydryl-to-sulfhydryl cross-linking chemistry. LC-MS^{E} characterization confirmed the modification of the CDR2 lysine residue by lysine-to-sulfhydryl cross-linking chemistry and an ELISA binding assay confirmed that the affinity of the V21H4-DOS47 produced by sulthydryl-to-sulfhydryl cross-linking chemistry was approximately six-fold stronger than that of the V21H1-DOS47 conjugate produced by lysine-to-sulfhydryl cross-linking chemistry.

Although the addition of a C-terminal cysteine residue proved extremely useful in the conjugation of V21H4-DOS47, it will be understood that, when working with other llama antibodies, it may be necessary to evaluate the status of any core cysteine residues before determining if this strategy can be used. This is because the sulfhydryl-to-sulfhydryl chemistry uniquely targets the C-terminal cysteine only because the core cysteine residues are joined in a disulfide bond, and thus unavailable for modification.

Protein refolding can be a slow and unreproducible process. Typically, refolding is performed by dilution or dialysis, and the process can take several days. In addition, yield is generally low (Yamaguchi and Miyazaki, 2014). The introduction of a DTT/cystamine redox couple led to a short and reproducible refolding process that generated high yields of active V21H4 antibody, which is useful for large scale production.

One benefit of conjugating antibodies to urease is the apparent increased affinity of the conjugate compared to antibody alone. By clustering multiple antibodies per urease, avidity increases as the relative off-rate of the complex is slower than for free antibody. However, the improvement in antibody avidity must be balanced by the potential detrimental effects of adding antibody to urease, including impairment of urease activity and increased immunogenicity of the conjugate. In addition, high conjugation ratios increase production costs and complexity. Each antibody-urease conjugate may have a different ideal conjugation ratio, as the availability of the target antigen differs and the orientation and activity of the antibody presented on the urease surface changes with different conjugation chemistries. In this study, we observed little improvement in antigen binding at conjugation ratios greater than 3.3. This is in contrast with L-DOS47, in which binding increased until eight antibodies were conjugated per urease. The use of a more flexible linker to generate V21H4-DOS47 compared to L-DOS47 may partially explain this difference, as the antibodies may be more accessible to target antigen. However, the difference between the two conjugates is most likely due to the fact that AFAIKL2, the antibody component of L-DOS47, has a much lower affinity for its target antigen than does V21 for VEGFR2 (data not shown). Thus, antibody multimerization has a more pronounced effect for AFAIKL2 than for V21.

### REFERENCES

Arbabi Ghahroudi. et al. (1997). FEBS Lett. 414, 521-526.
Cortez-Retamozo, et al. (2002). Int. J. Cancer. 98, 456-462.
Das, M., Wakelee, H. (2014). Transl. Lung Cancer Res. 3, 397-399.
De Genst, et al. (2006). Dev. Comp. Immunol. 30, 187-198.
Dumoulin, et al. (2002). Protein Sci. 11, 500-515.
Faivre et al. (2007). Nat. Rev. Drug Discov. 6, 734-745.
Frenken, et al. (2000). J. Biotechnol. 78, 11-21.
Guo, et al. (2010). Biochim. Biophys. Acta. 1806, 108-121.
Hanahan, D. and Folkman, J. (1996). Cell. 86, 353-364.
Harmsen, M. M., De Haard, H. J. (2007). Appl. Microbiol. Biotechnol. 77, 13-22.
Itakura, et al. (2000). Int. J. Cancer. 85, 27-34.
Lauwereys, et al. (1998). EMBO J. 17, 3512-3520.
Maass, et al. (2007). J. Immuno. Methods. 324, 13-25.
Muyldermans, et al. (2001) Trends Biochem. Sci. 26, 230-235.
Olsson, et al. (2006). Nat. Rev. Mol. Cell. Biol. 7, 359-371.
Ribatti, D. (2011). Int. J. Dev. Biol. 55, 383-388.
Risau, W. (1997). Nature. 386, 671-674.
Takishima, et al. (1988). Eur. J. Biochem. 175, 15-165.
Tanno, et al. (2004). Lung Cancer. 46, 11-19.
Thirumurugan, et al. (2013). Chem. Rev. 113, 4905-4979.
Tian, et al. (2015). Bioconjugate Chem. 26, 1144-1155.
van der Linden, et al. (1999). Biochim. Biophys. Acta. 1431, 37-46.
Wilhelm, et al. (2006). Nat. Rev. Drug Discov. 5, 835-844.
Wong, et al. (2005). J. Exp. Ther. Oncol. 5, 93-99.
Yamaguchi, H., Miyazaki, M. (2014). Biomolecules. 4, 235-251.

The foregoing description and examples have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance.

Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A polypeptide comprising the sequence of any one of SEQ ID NO:2-30 or a fragment or variant thereof.

2. The polypeptide of claim 1, wherein said polypeptide specifically binds to VEGFR-2.

3. The polypeptide of claim 2, wherein the polypeptide binds to an epitope of VEGFR-2.

4. The polypeptide of claim 1, wherein the fragments or variants have at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NO:2-30.

5. The polypeptide of claim 1, wherein said polypeptide is a single domain antibody.

6. The polypeptide of claim 4, wherein said polypeptide is selected from the group consisting of:
a polypeptide comprising the sequence of SEQ ID NO:2 or fragments or variants thereof having more than 93% identity to SEQ ID NO:2, or fragments or variants thereof having more than 85% identity to SEQ ID NO:2, wherein the fragments or variants thereof comprise more than 116 amino acid residues;
a polypeptide comprising the sequence of SEQ ID NO: 11 or fragments or variants thereof having more than 77% identity to SEQ ID NO: 11;
a polypeptide comprising the sequence of SEQ ID NO: 19 or fragments or variants thereof having more than 88% identity to SEQ ID NO: 19;
a polypeptide comprising the sequence of SEQ ID NO:6 or fragments or variants thereof having more than 86% identity to SEQ ID NO:6;
a polypeptide comprising the sequence of SEQ ID NO:25 or fragments or variants thereof having more than 80% identity to SEQ ID NO:25;
a polypeptide comprising the sequence of SEQ ID NO:26 or fragments or variants thereof having more than 80% identity to SEQ ID NO:26;
a polypeptide comprising the sequence of SEQ ID NO:30 or fragments or variants thereof having more than 80% identity to SEQ ID NO:30;
a polypeptide comprising the sequence of SEQ ID NO:8 or fragments or variants thereof having more than 80% identity to SEQ ID NO:8;
a polypeptide comprising the sequence of SEQ ID NO: 10 or fragments or variants thereof having more than 80% identity to SEQ ID NO: 10;
a polypeptide comprising the sequence of SEQ ID NO: 15 or fragments or variants thereof having more than 80% identity to SEQ ID NO: 15;
a polypeptide comprising the sequence of SEQ ID NO: 16 or fragments or variants thereof having more than 80% identity to SEQ ID NO: 16;
a polypeptide comprising the sequence of SEQ ID NO: 17 or fragments or variants thereof having more than 80% identity to SEQ ID NO: 17; and
A polypeptide comprising the sequence of SEQ ID NO:22 or fragments or variants thereof having more than 80% identity to SEQ ID NO:22.

7. The polypeptide of claim 1, further comprising a linker sequence and the linker optionally comprises a terminal cysteine.

8. The polypeptide of claim 7, wherein the linker sequence is selected from the group consisting of SEQ ID NO:54-69.

9. The polypeptide of claim 1, coupled to a fusion partner sequence.

10. The polypeptide of claim 9, wherein the fusion partner sequence comprises the sequence of SEQ ID NO:71 or a variant thereof.

11. A composition comprising the polypeptide, fragment, or variant of claim 1, optionally comprising a pharmaceutically acceptable carrier and/or a therapeutic agent.

12. An antibody or fragment thereof comprising the polypeptide of claim 1.

13. The antibody of claim 12, wherein the antibody or fragment thereof comprises at least one CDR having a sequence selected from the group consisting of SYAMG, AISWSDDSTYYANSVKG, HKSLQRPDEYTY and a sequence at least 70% identical thereto which binds VEGFR2.

14. The antibody or fragment of claim 12, wherein the antibody or fragment is a single domain antibody.

15. The antibody or fragment of claim 12, wherein the antibody or fragment specifically binds to VEGFR-2.

16. The antibody or fragment of claim 12, wherein the antibody or fragment specifically binds to a complex of VEGF and VEGFR-2.

17. The antibody or fragment of claim 12, wherein the antibody or fragment binds with a KD of less than 10⁻⁷ M.

18. The antibody or fragment of claim 12, wherein the antibody or fragment is humanized.

19. The antibody or fragment of claim 12, wherein the antibody or fragment is conjugated to another moiety.

20. The antibody or fragment of claim 19, wherein the antibody is linked to an Fc fragment.

21. The antibody or fragment of claim 20, wherein the Fc fragment is mouse Fc2b or human Fc1.

22. The antibody or fragment of claim 12, wherein the antibody or fragment is linked to a cargo molecule.

23. The antibody or fragment of claim 22, wherein the cargo molecule is a therapeutic molecule or a diagnostic agent.

24. The antibody or fragment of claim 12, wherein said antibody or fragment is of dromedary, camel, llama, or alpaca origin.

25. A composition comprising one or more of the antibodies or fragment or variants thereof of claim 12.

26. A single domain antibody comprising a polypeptide comprising the sequence of any one of SEQ ID NO:2-30 or a fragment or variant thereof.


SEQUENCE LISTING (77 sequences)

SEQ ID NO: 1 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 2 - protein, 122 aa, Lama glama
MQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSS

SEQ ID NO: 3 - protein, 133 aa, Lama glama
MQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEEEDDDGKK

SEQ ID NO: 4 - protein, 135 aa, Lama glama
MQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEQKGGGEEDDG

SEQ ID NO: 5 - protein, 148 aa, Lama glama
MKAIFVLKGSLDRDPEFDDEGGGQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEQ

SEQ ID NO: 6 - protein, 136 aa, Lama glama
MQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEQKGGGEEDDGC

SEQ ID NO: 7 - protein, 141 aa, Lama glama
MQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEQKLISEEDLNHHHHHH

SEQ ID NO: 8 - protein, 124 aa, Lama glama
MKKQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSS

SEQ ID NO: 9 - protein, 133 aa, Lama glama
MKKQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSSGSEEEDDDG

SEQ ID NO: 10 - protein, 144 aa, Lama glama
MKAIFVLKGSLDRDPEFDDEGGGQVQLVESGGGLVQAGGSLRLSCAASGRAFSSYAMGWFRQAPGKERELVAAISWSDDSTYYANSVKGRFTISRDNAKSAVYLQMNSLKPEDTAVYYCAAHKSLQRPDEYTYWGQGTQVTVSS

SEQ ID NO: 11 - protein, 126 aa, Homo sapiens
MQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSS

SEQ ID NO: 12 - protein, 136 aa, Homo sapiens
MQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSSGSEEDDDEEK

SEQ ID NO: 13 - protein, 144 aa, Homo sapiens
MQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSSGSEQKLISEEDLNHHHHH

SEQ ID NO: 14 - protein, 149 aa, Homo sapiens
MQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSSGSEQKLISEEDLNGGGEDDEEGC

SEQ ID NO: 15 - protein, 125 aa, Homo sapiens
QVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSS

SEQ ID NO: 16 - protein, 143 aa, Homo sapiens
QVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSSGSEQKLISEEDLNHHHHH

SEQ ID NO: 17 - protein, 149 aa, Homo sapiens
MKAIFVLKGSLDRDPEFDDEEGGGQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSS

SEQ ID NO: 18 - protein, 154 aa, Homo sapiens
MKAIFVLKGSLDRDPEFDDEEGGGQVQLVESGGGLIKPGGSLRLSCAASGFRFSAESMTWVRQAPGKGLEWVSAISSSGGSTYYADSVKGRFTISRDNSKNTVYLQMNSLRAEDTAVYYCVRSPKGCTHASCSWNSGSWGQGTLVTVSSGSDEE

SEQ ID NO: 19 - protein, 126 aa, Homo sapiens
MQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSS

SEQ ID NO: 20 - protein, 140 aa, Homo sapiens
MQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSSGSEQKGGGEEDDEE

SEQ ID NO: 21 - protein, 144 aa, Homo sapiens
MQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSSGSEQKLISEEDLNHHHHH

SEQ ID NO: 22 - protein, 128 aa, Homo sapiens
MKKQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSS

SEQ ID NO: 23 - protein, 137 aa, Homo sapiens
MKKQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSSGSEEEDDDG

SEQ ID NO: 24 - protein, 147 aa, Homo sapiens
MQVQLVESGGGLIKPGGSLRLSCAASGDMLSYDVMSWVRQAPGKGLEWVSAISSSGGSTYYADSVKGRFTISRDNSKNTVYLQMNSLRAEDTAVYYCVAAPWRCTHDNCSKTRASWGQGTMVTVSSGSEQKLISEEDLNGGGEDEGC

SEQ ID NO: 25 - protein, 132 aa, Lama glama
MQVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSS

SEQ ID NO: 26 - protein, 136 aa, Lama glama
MQVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSSGSEQ

SEQ ID NO: 27 - protein, 151 aa, Lama glama
MQVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSSGSEQKLISEEDLNHHHHHH

SEQ ID NO: 28 - protein, 131 aa, Lama glama
QVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSS

SEQ ID NO: 29 - protein, 150 aa, Lama glama
QVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSSGSEQKLISEEDLNHHHHHH

SEQ ID NO: 30 - protein, 145 aa, Lama glama
MQVKLEESGGGLVQAGGSLRLSCAASGGTASSYAMGWFRQAPGKEREFVAAISRSGGNTDYVDSAKGRFTISRDDAKNTVSLQMNSLRLEDTAVYYCAARYAGTWPNDAGTVYWLPPNYNYWGQGTQVTVSSGSEQKGGGDEDGC

SEQ ID NO: 31 - DNA, 366 bp, Lama glama
atgcaggtgcagctggtggaatccggcggcggcctggtgcaggcgggcggctccctgcgtctgtcctgcgcggcgtccggccgtgcgttttcctcctatgcgatgggctggtttcgtcaggcgccgggcaaagaacgtgaactggtggcggcgatttcctggtccgatgattccacctattatgcgaattccgtgaaaggccgttttaccatttcccgtgataatgcgaaatccgcggtgtatctacagatgaattccctgaaaccggaagataccgcggtgtattattgcgcggcgcataaatccctacagcgtccggatgaatatacctattggggccagggcacccaggtgaccgtgtcctcc

SEQ ID NO: 32 - DNA, 366 bp, Lama glama
atgcaggtgcagcttgtggagtccggcggaggtcttgtccaggcaggagggtctttgcgcctgagctgcgcggcgagtgggcgcgcgttcagcagttacgcgatgggttggttccgccaggcccctgggaaagagcgtgaacttgtggctgccatttcttggtctgatgattccacctattatgctaattcagttaagggccgtttcacgattagccgcgataatgctaaatccgccgtctatcttcagatgaacagccttaagcctgaagatacggcagtatattattgtgccgctcataagagtctgcaacgcccggacgaatatacatactggggacagggcacgcaagttaccgtttccagc

SEQ ID NO: 33 - DNA, 405 bp, Lama glama
atgcaggtgcagctggtggaatccggcggcggcctggtgcaggcgggcggctccctgcgtctgtcctgcgcggcgtccggccgtgcgttttcctcctatgcgatgggctggtttcgtcaggcgccgggcaaagaacgtgaactggtggcggcgatttcctggtccgatgattccacctattatgcgaattccgtgaaaggccgttttaccatttcccgtgataatgcgaaatccgcggtgtatctacagatgaattccctgaaaccggaagataccgcggtgtattattgcgcggcgcataaatccctacagcgtccggatgaatatacctattggggccagggcacccaggtgaccgtgtcctccggctccgaacagaaaggcggcggcgaagaagatgatggc

SEQ ID NO: 34 - DNA, 423 bp, Lama glama
atgcaggtgcaactggttgaatcaggtggaggactggtgcaggccgggggatctttacgcttatcatgtgcagcttcggggcgtgccttctcctcttatgcgatgggatggttccgccaagcccccggcaaggagcgtgagctggtagcagccatttcctggtcagacgacagtacctactacgcaaactcagtcaaagggcgcttcactatctctcgcgacaatgccaaatccgctgtgtacttgcaaatgaactcattgaagccagaggatacggctgtctattactgtgcagcccacaagagtttacagcgtccagatgaatacacctattggggacaaggtacacaagttaccgttagttcgggtagcgaacaaaagttgatctctgaggaggacttaaatcatcatcatcatcaccat

SEQ ID NO: 35 - DNA, 408 bp, Lama glama
atgcaggtgcagcttgtggagtccggcggaggtcttgtccaggcaggagggtctttgcgcctgagctgcgcggcgagtgggcgcgcgttcagcagttacgcgatgggttggttccgccaggcccctgggaaagagcgtgaacttgtggctgccatttcttggtctgatgattccacctattatgctaattcagttaagggccgtttcacgattagccgcgataatgctaaatccgccgtctatcttcagatgaacagccttaagcctgaagatacggcagtatattattgtgccgctcataagagtctgcaacgcccggacgaatatacatactggggacagggcacgcaagttaccgtttccagcggttctgaacagaaaggaggcggtgaagaggatgatggctgc

SEQ ID NO: 36 - DNA, 432 bp, Lama glama
atgaaagcgatcttcgttctgaaaggttctctggaccgtgacccggaattcgacgacgaaggtggtggtcaggttcagctggttgaatctggtggtggtctggttcaggcgggtggttctctgcgtctgtcttgcgcggcgtctggtcgtgcgttctcttcttacgcgatgggttggttccgtcaggcgccgggtaaagaacgtgaactggttgcggcgatctcttggtctgacgactctacctactacgcgaactctgttaaaggtcgtttcaccatctctcgtgacaacgcgaaatctgcggtttacctacagatgaactctctgaaaccggaagacaccgcggtttactactgcgcggcgcacaaatctctacagcgtccggacgaatacacctactggggtcagggtacccaggttaccgtttcttct

SEQ ID NO: 37 - DNA, 444 bp, Lama glama
atgaaagcgatcttcgttctgaaaggttctctggaccgtgacccggaattcgacgacgaaggtggtggtcaggttcagctggttgaatctggtggtggtctggttcaggcgggtggttctctgcgtctgtcttgcgcggcgtctggtcgtgcgttctcttcttacgcgatgggttggttccgtcaggcgccgggtaaagaacgtgaactggttgcggcgatctcttggtctgacgactctacctactacgcgaactctgttaaaggtcgtttcaccatctctcgtgacaacgcgaaatctgcggtttacctacagatgaactctctgaaaccggaagacaccgcggtttactactgcgcggcgcacaaatctctacagcgtccggacgaatacacctactggggtcagggtacccaggttaccgtttcttctggttctgaacag

SEQ ID NO: 38 - DNA, 378 bp, Homo sapiens
atgcaagttcagttagtagaaagtggtggtggtttaatcaaaccgggtggttcacttcgtttatcgtgcgcagcaagcgggtttcgtttttcagcagaatcaatgacatgggttcgtcaagcaccgggcaaaggtttagagtgggtttcagcaatttcatcaagtggcggttcaacttattatgcagattcggttaaaggtcgtttcacaatttctcgcgataactcaaaaaatacggtttatttacaaatgaattccttacgtgcagaagatacagcagtttattattgtgttcgttctccaaaaggttgtactcacgcatcttgtagttggaatagtggtagttggggtcaaggtacattagttacagtctcaagc

SEQ ID NO: 39 - DNA, 378 bp, Homo sapiens
atgcaggtgcagttagttgagtcgggcgggggtcttattaaaccaggtggaagccttcgtctgtcttgtgcagcatcaggctttcgtttttccgcggaaagcatgacctgggtacgccaagcgcctggcaaaggattggagtgggtttcggccatttcttcttcaggaggatcaacgtactatgcagactccgtaaaaggacgcttcacgatttctcgcgataactctaagaacaccgtgtacttacaaatgaactctttacgtgcagaggacacagcagtgtattattgtgttcgctcacccaaaggctgcacccatgcgtcatgctcttggaactcaggttcgtggggccaggggaccttggtgacagtatcctcg

SEQ ID NO: 40 - DNA, 408 bp, Homo sapiens
atgcaagttcagttagtagaaagtggtggtggtttaatcaaaccgggtggttcacttcgtttatcgtgcgcagcaagcgggtttcgtttttcagcagaatcaatgacatgggttcgtcaagcaccgggcaaaggtttagagtgggtttcagcaatttcatcaagtggcggttcaacttattatgcagattcggttaaaggtcgtttcacaatttctcgcgataactcaaaaaatacggtttatttacaaatgaattccttacgtgcagaagatacagcagtttattattgtgttcgttctccaaaaggttgtactcacgcatcttgtagttggaatagtggtagttggggtcaaggtacattagttacagtctcaagcggttcagaagaagatgacgatgaagaaaaa

SEQ ID NO: 41 - DNA, 432 bp, Homo sapiens
atgcaggtgcagttagttgagtcgggcgggggtcttattaaaccaggtggaagccttcgtctgtcttgtgcagcatcaggctttcgtttttccgcggaaagcatgacctgggtacgccaagcgcctggcaaaggattggagtgggtttcggccatttcttcttcaggaggatcaacgtactatgcagactccgtaaaaggacgcttcacgatttctcgcgataactctaagaacaccgtgtacttacaaatgaactctttacgtgcagaggacacagcagtgtattattgtgttcgctcacccaaaggctgcacccatgcgtcatgctcttggaactcaggttcgtggggccaggggaccttggtgacagtatcctcgggctccgaacagaagttaattagtgaagaagatttgaaccaccaccaccatcac

SEQ ID NO: 42 - DNA, 447 bp, Homo sapiens
atgaaagcgatcttcgttctgaaaggttctctggaccgtgacccggaattcgacgacgaagaaggtggtggtcaggttcagctggttgaatctggtggtggtctgatcaaaccgggtggttctctgcgtctgtcttgcgcggcgtctggtttccgtttctctgcggaatctatgacctgggttcgtcaggcgccgggtaaaggtctggaatgggtttctgcgatctcttcttctggtggttctacctactacgcggactctgttaaaggtcgtttcaccatctctcgtgacaactctaaaaacaccgtttacttacaaatgaactctctgcgtgcggaagacaccgcggtttactactgcgttcgttctccgaaaggttgcacccacgcgtcttgctcttggaactctggttcttggggtcagggtaccctggttaccgtttcttct

SEQ ID NO: 43 - DNA, 462 bp, Homo sapiens
atgaaagcgatcttcgttctgaaaggttctctggaccgtgacccggaattcgacgacgaagaaggtggtggtcaggttcagctggttgaatctggtggtggtctgatcaaaccgggtggttctctgcgtctgtcttgcgcggcgtctggtttccgtttctctgcggaatctatgacctgggttcgtcaggcgccgggtaaaggtctggaatgggtttctgcgatctcttcttctggtggttctacctactacgcggactctgttaaaggtcgtttcaccatctctcgtgacaactctaaaaacaccgtttacttacaaatgaactctctgcgtgcggaagacaccgcggtttactactgcgttcgttctccgaaaggttgcacccacgcgtcttgctcttggaactctggttcttggggtcagggtaccctggttaccgtttcttctggttctgacgaagaa

SEQ ID NO: 44 - DNA, 372 bp, Homo sapiens
atgcaggtgcagctggtggaaagcggcggcggcctgattaaaccgggcggcagcctgcgcctgagctgcgcggcgagcggcgatatgctgagctatgatgtgatgagctgggtgcgccaggcgccgggcaaaggcctggaatgggtgagcgcgattagcagcagcggcggcagcacctattatgcggatagcgtgaaaggccgctttaccattagccgcgataacagcaaaaacaccgtgtatcttcagatgaacagcctgcgcgcggaagataccgcggtgtattattgcgtggcggcgccgtggcgctgcacccatgataactgctctaaaacccgcgcgagctggggccagggcaccatggtgaccgtg

SEQ ID NO: 45 - DNA, 378 bp, Homo sapiens
atgcaagtacagttagtggagagtggaggagggctgattaagccaggcggctctttgcgtctgagttgtgcggcatcaggcgatatgttaagctacgatgtgatgagttgggtgcgtcaagcgccaggaaaaggacttgaatgggtcagcgcaatttcgtcgtccggtgggtctacttactacgctgattcggttaagggccgcttcaccatctcccgcgacaattcaaagaatacggtatatctgcaaatgaatagtttgcgtgcggaggacacagcagtctactattgcgttgcagctccctggcgctgtactcacgataactgttcaaaaacccgcgcatcatggggtcaaggtacaatggtgacagtgtcatct

SEQ ID NO: 46 - DNA, 420 bp, Homo sapiens
atgcaggtgcagctggtggaaagcggcggcggcctgattaaaccgggcggcagcctgcgcctgagctgcgcggcgagcggcgatatgctgagctatgatgtgatgagctgggtgcgccaggcgccgggcaaaggcctggaatgggtgagcgcgattagcagcagcggcggcagcacctattatgcggatagcgtgaaaggccgctttaccattagccgcgataacagcaaaaacaccgtgtatcttcagatgaacagcctgcgcgcggaagataccgcggtgtattattgcgtggcggcgccgtggcgctgcacccatgataactgctctaaaacccgcgcgagctggggccagggcaccatggtgaccgtgagcagcggcagcgaacagaaaggcggcggcgaagaagatgatgaagaa

SEQ ID NO: 47 - DNA, 432 bp, Homo sapiens
atgcaagtacagttagtggagagtggaggagggctgattaagccaggcggctctttgcgtctgagttgtgcggcatcaggcgatatgttaagctacgatgtgatgagttgggtgcgtcaagcgccaggaaaaggacttgaatgggtcagcgcaatttcgtcgtccggtgggtctacttactacgctgattcggttaagggccgcttcaccatctcccgcgacaattcaaagaatacggtatatctgcaaatgaatagtttgcgtgcggaggacacagcagtctactattgcgttgcagctccctggcgctgtactcacgataactgttcaaaaacccgcgcatcatggggtcaaggtacaatggtgacagtgtcatctggtagtgaacagaagttaattagtgaagaggaccttaatcatcatcatcatcac

SEQ ID NO: 48 - DNA, 441 bp, Homo sapiens
atgcaggttcagctggttgaatctggtggtggtctgatcaaaccgggtggttctctgcgtctgtcttgcgcggcgtctggtgacatgctgtcttacgacgttatgtcttgggttcgtcaggcgccgggtaaaggtctggaatgggtttctgcgatctcttcttctggtggttctacctactacgcggactctgttaaaggtcgtttcaccatctctcgtgacaactctaaaaacaccgtttacctgcaaatgaactctctgcgtgcggaagacaccgcggtttactactgcgttgcggcgccgtggcgttgcacccacgacaactgctctaaaacccgtgcgtcttggggtcagggtaccatggttaccgtttcttctggttctgaacagaaactgatctctgaagaagacctgaacggtggtggtgaagacgaaggttgc

SEQ ID NO: 49 - DNA, 396 bp, Lama glama
atgcaagtaaaactcgaagaatcaggtggaggattggttcaagctggtgggtcattacgtttgtcctgtgcagcaagtggcggtactgcgtcaagttatgcaatgggttggtttcgtcaagctcccggtaaagaacgtgaatttgttgccgcaattagtcggtccggaggaaatacagattatgtagactcagcaaaaggtcgttttactatctcacgcgatgatgcaaaaaatacggtttccttacaaatgaactctctgcgcctcgaagataccgcggtatattattgcgctgcccgctacgccggtacctggccgaatgatgctggcactgtatattggctgccaccgaattacaactattggggtcaaggaactcaagtcacggtaagcagc

SEQ ID NO: 50 - DNA, 396 bp, Lama glama
atgcaggttaaattagaggaatcaggtggaggtttggttcaagcaggtggtagcttgcgcctgagttgtgccgctagcgggggcacagccagttcatacgcgatggggtggtttcgccaggcccctggaaaggagcgtgaattcgttgctgcgattagtcgtagcggcggtaacacggattacgtggacagcgcgaagggacgctttacaatttctcgtgatgacgcaaagaacacggtgtccctgcaaatgaactcacttcgcctggaagacaccgcggtgtattattgtgcagcccgctacgcgggaacttggccgaacgatgctggtaccgtgtactggttaccccctaattacaattactggggccaaggtacccaagtcaccgtctcctcg

SEQ ID NO: 51 - DNA, 438 bp, Lama glama
atgcaagtaaaactcgaagaatcaggtggaggattggttcaagctggtgggtcattacgtttgtcctgtgcagcaagtggcggtactgcgtcaagttatgcaatgggttggtttcgtcaagctcccggtaaagaacgtgaatttgttgccgcaattagtcggtccggaggaaatacagattatgtagactcagcaaaaggtcgttttactatctcacgcgatgatgcaaaaaatacggtttccttacaaatgaactctctgcgcctcgaagataccgcggtatattattgcgctgcccgctacgccggtacctggccgaatgatgctggcactgtatattggctgccaccgaattacaactattggggtcaaggaactcaagtcacggtaagcagcggttccgaacaaaagggtggtggagaagaagatgatggcaaa

SEQ ID NO: 52 - DNA, 453 bp, Lama glama
atgcaggttaaattagaggaatcaggtggaggtttggttcaagcaggtggtagcttgcgcctgagttgtgccgctagcgggggcacagccagttcatacgcgatggggtggtttcgccaggcccctggaaaggagcgtgaattcgttgctgcgattagtcgtagcggcggtaacacggattacgtggacagcgcgaagggacgctttacaatttctcgtgatgacgcaaagaacacggtgtccctgcaaatgaactcacttcgcctggaagacaccgcggtgtattattgtgcagcccgctacgcgggaacttggccgaacgatgctggtaccgtgtactggttaccccctaattacaattactggggccaaggtacccaagtcaccgtctcctcgggaagcgaacaaaagctgattagcgaagaggatcttaaccatcatcatcaccatcac

SEQ ID NO: 53 - DNA, 435 bp, Lama glama
atgcaggttaaactggaagaatctggtggtggtctggttcaggcgggtggttctctgcgtctgtcttgcgcggcgtctggtggtaccgcgtcttcttacgcgatgggttggttccgtcaggcgccgggtaaagaacgtgaattcgttgcggcgatctctcgttctggtggtaacaccgactacgttgactctgcgaaaggtcgtttcaccatctctcgtgacgacgcgaaaaacaccgtttctctgcaaatgaactctctgcgtctggaagacaccgcggtttactactgcgcggcgcgttacgcgggtacctggccgaacgacgcgggtaccgtttactggctgccgccgaactacaactactggggtcagggtacccaggttaccgtttcttctggttctgaacagaaaggtggtggtgacgaagacggttgc

SEQ ID NO: 54 - protein, 4 aa, Artificial Sequence
  Linker
GSEQ

SEQ ID NO: 55 - protein, 5 aa, Artificial Sequence
  A synthetic linker
GSDEE

SEQ ID NO: 56 - protein, 9 aa, Artificial Sequence
  A synthetic linker
GSEEEDDDG

SEQ ID NO: 57 - protein, 11 aa, Artificial Sequence
  A synthetic linker
GSEEEDDDGKK

SEQ ID NO: 58 - protein, 13 aa, Artificial Sequence
  A synthetic linker
GSEQKGGGEEDDG

SEQ ID NO: 59 - protein, 18 aa, Artificial Sequence
  A synthetic linker
GSEQKLISEEDLNHHHHH

SEQ ID NO: 60 - protein, 19 aa, Artificial Sequence
  A synthetic linker
GSEQKLISEEDLNHHHHHH

SEQ ID NO: 61 - protein, 10 aa, Artificial Sequence
  A synthetic linker
GSEEDDDEEK

SEQ ID NO: 62 - protein, 14 aa, Artificial Sequence
  A synthetic linker
GSEQKGGGEEDDEE

SEQ ID NO: 63 - protein, 22 aa, Artificial Sequence
  A synthetic linker
GSEQKLISEEDLNGGGEDDEEG

SEQ ID NO: 64 - protein, 20 aa, Artificial Sequence
  A synthetic linker
GSEQKLISEEDLNGGGEDEG

SEQ ID NO: 65 - protein, 12 aa, Artificial Sequence
  A synthetic linker
GSEQKGGGDEDG

SEQ ID NO: 66 - protein, 14 aa, Artificial Sequence
  A synthetic linker
GSEQKGGGEEDDGC

SEQ ID NO: 67 - protein, 23 aa, Artificial Sequence
  A synthetic linker
GSEQKLISEEDLNGGGEDDEEGC

SEQ ID NO: 68 - protein, 21 aa, Artificial Sequence
  A synthetic linker
GSEQKLISEEDLNGGGEDEGC

SEQ ID NO: 69 - protein, 13 aa, Artificial Sequence
  A synthetic linker
GSEQKGGGDEDGC

SEQ ID NO: 70 - protein, 8 aa, Artificial Sequence
  A synthetic flag tag
DYKDDDDK

SEQ ID NO: 71 - protein, 20 aa, Artificial Sequence
  A synthetic fusion partner
MKAIFVLKGSLDRDPEFDDE

SEQ ID NO: 72 - protein, 9 aa, Artificial Sequence
  A synthetic variable modifier
  MISC_FEATURE (9)..(9): BM(PEG)2 modifed residue
GGGEEDDGC

SEQ ID NO: 73 - protein, 9 aa, Artificial Sequence
  A synthetic cross-linker activated peptide
  MISC_FEATURE (9)..(9): BM(PEG)2-Cys modified residue
GGGEEDDGC

SEQ ID NO: 74 - protein, 8 aa, Artificial Sequence
  A synthetic cross-linker activated peptide
  MISC_FEATURE (8)..(8): BM(PEG)2-Cys modified residue
LSCAASGR

SEQ ID NO: 75 - protein, 24 aa, Artificial Sequence
  A synthetic cross-linker activated peptide
  MISC_FEATURE (24)..(24): BM(PEG)2-Cys modified residue
SAVYLQMNSLKPEDTAVYYCAAHK

SEQ ID NO: 76 - protein, 9 aa, Artificial Sequence
  A synthetic linker
GGGEEDDGC

SEQ ID NO: 77 - protein, 14 aa, Canavalia ensiformis
LLCVSEATTVPLSR